SEQUENCE LISTING (10 sequences)

SEQ ID NO: 1 - protein, 146 aa, Artificial sequence
  Synthetic heavy chain variable region 1H12VH
MGFKMESQFQVFVFVFLWLSGVDGEVQLVESGGDLVKPGGSLKLSCAASGFTFSSYGMSWVRQTPDKRLEWVATISSGGSYTYYPDSVKGRFTISRDNAKNTLYLQMSSLKSEDTAMYYCARHPIYYTYDDTMDYWGQGTSVTVSS

SEQ ID NO: 2 - protein, 145 aa, Artificial sequence
  Synthetic heavy chain variable region 1H12RHA
MGFKMESQFQVFVFVFLWLSGVDGQVQLVESGGGVVQPGRSLRLSCAASGFTFSSYGMSVRQAPGKGLEWVATISSGGSYTYYPDSVKGRFTISRDNSKNTLYLQMNSLRAEDTAVYYCARHPIYYTYDDTMDYWGQGTTVTVSS

SEQ ID NO: 3 - protein, 146 aa, Artificial sequence
  Synthetic heavy chain variable region 1H12RHB
MGFKMESQFQVFVFVFLWLSGVDGEVQLVESGGGLVQPGGSLRLSCAASGFTFSSYGMSWVRQAPGKGLEWVATISSGGSYTYYPDSVKGRFTISRDNAKNSLYLQMNSLRAEDTAVYYCARHPIYYTYDDTMDYWGQGTLVTVSS

SEQ ID NO: 4 - protein, 132 aa, Artificial sequence
  Synthetic light chain variable region 1H12VK
MGFKMESQFQVFVFVFLWLSGVDGENVLTQSPAIMAASPGEKVTMTCSASSSVSSGNFHWYQQKPGTSPKLWIYRTSNLASGVPARFSGSGSGTSYSLTISSMEAEDAATYYCQQWSGYPWTFGGGTKLEIK

SEQ ID NO: 5 - protein, 132 aa, Artificial sequence
  Synthetic light chain variable region 1H12RKA
MGFKMESQFQVFVFVFLWLSGVDGEIVLTQSPATLSLSPGERATLSCSASSSVSSGNFHWYQQKPGLAPRLLIYRTSNLASGIPDRFSGSGSGTDFTLTISRLEPEDFAVYYCQQWSGYPWTFGPGTKVDIK

SEQ ID NO: 6 - protein, 132 aa, Artificial sequence
  Synthetic light chain variable region 1H12RKA1
MGFKMESQFQVFVFVFLWLSGVDGENVLTQSPATLSLSPGERATLSCSASSSVSSGNFHWYQQKPGLAPRLWIYRTSNLASGIPDRFSGSGSGTDYTLTISRLEPEDFAVYYCQQWSGYPWTFGPGTKVDIK

SEQ ID NO: 7 - protein, 132 aa, Artificial sequence
  Synthetic light chain variable region 1H12RKB
MGFKMESQFQVFVFVFLWLSGVDGEIVLTQSPGTLSLSPGERATLSCSASSSVSSGNFHWYQQKPGLAPRLLIYRTSNLASGIPARFSGSGSGTDFTLTISSLEPEDFAVYYCQQWSGYPWTFGGGTKLEIK

SEQ ID NO: 8 - protein, 132 aa, Artificial sequence
  Synthetic light chain variable region 1H12RKB1
MGFKMESQFQVFVFVFLWLSGVDGENVLTQSPGTLSLSPGERATLSCSASSSVSSGNFHWYQQKPGLAPRLWIYRTSNLASGIPARFSGSGSGTDYTLTISSLEPEDFAVYYCQQWSGYPWTFGGGTKLEIK

SEQ ID NO: 9 - protein, 894 aa, Homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRRGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 10 - protein, 888 aa, Mus musculus
MGRVPLAWWLALCCWGCAAHKDTQTEAGSPFVGNPGNITGARGLTGTLRCELQVQGEPPEVVWLRDGQILELADNTQTQVPLGEDWQDEWKVVSQLRISALQLSDAGEYQCMVHLEGRTFVSQPGFVGLEGLPYFLEEPEDKAVPANTPFNLSCQAQGPPEPVTLLWLQDAVPLAPVTGHSSQHSLQTPGLNKTSSFSCEAHNAKGVTTSRTATITVLPQRPHHLHVVSRQPTELEVAWTPGLSGIYPLTHCNLQAVLSDDGVGIWLGKSDPPEDPLTLQVSVPPHQLRLEKLLPHTPYHIRISCSSSQGPSPWTHWLPVETTEGVPLGPPENVSAMRNGSQVLVRWQEPRVPLQGTLLGYRLAYRGQDTPEVLMDIGLTREVTLELRGDRPVANLTVSVTAYTSAGDGPWSLPVPLEPWRPGQGQPLHHLVSEPPPRAFSWPWWYVLLGALVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSDREGFPEPVVILPFMKHGDLHSFLLYSRLGDQPVFLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPVDCLDGLYALMSRCWELNPRDRPSFAELREDLENTLKALPPAQEPDEILYVNMDEGGSHLEPRGAAGGADPPTQPDPKDSCSCLTAADVHSAGRYVLCPSTAPGPTLSADRGCPAPPGQEDGA

(19) United States
(12) Patent Application Publication
(10) Pub. No.: US 2018/0127505 A1
(43) Pub. Date: May 10, 2018

(54) HUMANIZED ANTI-AXL ANTIBODIES AND THEIR CONJUGATES

(71) Applicants: MEDIMMUNE LIMITED, Cambridge, Cambridgeshire (GB); ADC THERAPEUTICS S.A., EPALINGES (CH)

(72) Inventors: PATRICIUS HENDRIKUS CORNELIS VAN BERKEL, Lausanne (CH); PHILIP WILSON HOWARD, Cambridge (GB); DAVID G. WILLIAMS, Epson (GB)

(21) Appl. No.: 15/566,639

(22) PCT Filed: Apr. 15, 2016
(86) PCT No.: PCT/EP2016/058374

§ 371 Date: Oct. 13, 2017

(30) Foreign Application Priority Data
Apr. 15, 2015 (GB) 1506409.0

(51) Int. Cl. C07K 16/28; A61K 47/68; A61P 35/00

(52) U.S. Cl. CPC C07K 16/2863; A61K 47/6851; C07K 2317/24; A61P 35/00; A61K 47/6803

(57) ABSTRACT

The present disclosure relates to humanized anti-Axl antibodies and conjugates thereof. Conjugates comprising pyrroiobenzodiazepines (PBDs) having a labile protecting group in the form of a linker to the antibody are described.

The present disclosure relates to humanized anti-Axl antibodies and conjugates thereof. Conjugates comprising pyrrolobenzodiazepines (PBDs) having a labile protecting group in the form of a linker to the antibody are described.

### BACKGROUND

Axl

Axl is a member of the TAM (Tyro3-Axl-Mer) receptor tyrosine kinases (RTK) that share the vitamin K-dependent ligand Gas6 (growth arrest-specific 6). TAM family RTKs regulate a diverse range of cellular responses including cell survival, proliferation, autophagy, migration, angiogenesis, platelet aggregation, and natural killer cell differentiation. Axl is expressed in many embryonic tissues and is thought to be involved in mesenchymal and neural development, with expression in adult tissues largely restricted to smooth muscle cells (MGI Gene Expression Database; www.informatics.jax.org). Axl activation is linked to several signal transduction pathways, including Akt, MAP kinases, NF-κB, STAT, and others. Originally identified as a transforming gene from a patient with chronic myelogenous leukaemia, Axl has since been associated with various high-grade cancers and correlated with poor prognosis.

Axl receptor overexpression has been detected in a wide range of solid tumours and myeloid leukaemia (Linger et al, Adv Cancer Res. 100: 35, 2008; Linger et al, Expert Opin Ther Targets. 14:1073, 2010).

Axl expression correlates with malignant progression and is an independent predictor of poor patient overall survival in several malignancies including pancreatic (Song et al, Cancer. 117:734, 2011), prostate (Paccez et al, Oncogene. 32:698, 2013), lung (Ishikawa et al. Ann Surg Oncol. 2012; Zhang et al, Nat Genet. 44:852, 2012), breast (Gjerdrum, Proc natl Acad Sci USA 107:1124, 2010), colon cancer (Yuen et al, PLoS One, 8:e54211, 2013) and acute myeloid leukaemia (AML) (Ben-Batalla et al, Blood 122:2443, 2013).

Axl signal transduction is activated by a protein ligand (Gas6) secreted by tumour associated macrophages (Loges et al, Blood. 115:2264, 2010) or autocrine mechanisms (Gjerdrum, Proc natl Acad Sci USA 107:1124, 2010), that drives receptor dimerization, autophosphorylation and downstream signalling, such as via PI3 kinase (PI3K)-AKT, particularly AKT and mitogen-activated protein kinase (MAPK) pathways (Korshunov, Clinical Science. 122:361, 2012). Heterodimerization with other tyrosine kinase receptors, e.g. epidermal growth factor receptor (EGFR), is also reported to occur (Linger et al, Expert Opin Ther Targets. 14:1073, 2010; Meyer et al Science Signalling 6:ra66, 2013).

Aberrant activation of Axl in tumour cells is widely associated with acquired drug resistance to targeted therapeutics in vitro and in vivo (Zhang et al. Nat Genet. 44: 852, 2012; Byers et al. Clin Cancer Res. 19: 279, 2013). Axl-targeting agents block tumour formation, metastasis and reverse drug resistance (e.g. to erlotinib) by reversing EMT/CSC characteristics in several experimental cancer models, including triple negative breast cancer, hormone resistant prostate cancer and adenocarcinoma of the lung (Holland et al Cancer Res 70:1544, 2010; Gjerdrum, Proc natl Acad Sci USA 107:1124, 2010; Zhang et al. Nat Genet. 44: 852, 2012; Paccez et al, Oncogene. 32:698, 2013).

Anti-Axl Antibodies

applications relating to Axl and anti-Axl antibodies include EP2267454A2 [Diagnosis and prevention of cancer cell invasion measuring . . . Axl-Max Planck]; WO02009063965 [anti Axl—Chugai Pharmaceutical]; WO2011159980A1 [anti-Axl-Genentech], WO2011014457A1 [combination treatments Axl and VEGF antagonists—Genentech]; WO2012-175691A1 [Anti Axl 20G7-D9—INSERM], WO2012-175692A1 [Anti Axl 3E3E8—INSERM];

WO02009/062690A1 [anti Axl—U3 Pharma] and WO02010/130751A1 [humanised anti Axl—U3 Pharma].

GB1410826.0 discloses the murine anti-AxI antibody designated herein as “mouse 1H12”. In view of the advantageous properties of this antibody and its potential clinical applications in humans, it is desirable to identify humanised versions of the murine antibody which have reduced immunogenicity to humans. The present disclosure concerns such antibodies, along with antibody-drug conjugates comprising the humanised 1H12 antibodies and PBD drug-moieties.

### SUMMARY

The present disclosure provides humanized anti-AXL antibodies derived from the ‘mouse 1H12’ antibody, and conjugates thereof.

The present inventors have generated a number of humanised heavy chain variable regions (SEQ ID NOs: 2 and 3) and humanised light chain variable regions (SEQ ID NOs: 5 to 8) with a view to creating antibodies that have lower immunogenicity in a human individual than the ‘mouse 1H12’ antibody or ‘chimeric 1H12’ antibody whilst retaining antigen-binding potency.

Surprisingly, these humanised antibodies have also been found to have other advantageous properties, such as increased charge at physiological pH and improved affinity for some Axl ligands.

Accordingly, in one aspect the present disclosure comprises an isolated humanized antibody that binds to AXL, wherein the isolated humanized antibody comprises a heavy chain variable region having the amino acid sequence of SEQ ID NO: 1, 2, or 3. In some embodiments the antibody further comprises a light chain variable region having the amino acid sequence of SEQ ID NO: 4, 5, 6, 7, or 8 and, optionally, further comprises a constant region derived from one or more human antibodies.

In some embodiments the isolated humanized antibody that binds to AXL comprises; a heavy chain variable region having the amino acid sequence of SEQ ID NO: 2 or 3; a light chain variable region having the amino acid sequence of SEQ ID NO: 5, 6, 7, or 8; and, optionally, comprises a constant region derived from one or more human antibodies.

In some embodiments, the humanized antibody does not comprise a heavy chain variable region having the amino acid sequence of SEQ ID NO: 1 and a light chain variable region having the amino acid sequence of SEQ ID NO: 4.

In some embodiments the isolated humanized antibody that binds to AXL comprises:

(i) a heavy chain variable region having the amino acid sequence of SEQ ID NO: 1 and a light chain variable region having the amino acid sequence of SEQ ID NO: 4;

(ii) a heavy chain variable region having the amino acid sequence of SEQ ID NO: 1 and a light chain variable region having the amino acid sequence of SEQ ID NO: 5;

(iii) a heavy chain variable region having the amino acid sequence of SEQ ID NO: 1 and a light chain variable region having the amino acid sequence of SEQ ID NO: 6;

(iv) a heavy chain variable region having the amino acid sequence of SEQ ID NO: 1 and a light chain variable region having the amino acid sequence of SEQ ID NO: 7;

(v) a heavy chain variable region having the amino acid sequence of SEQ ID NO: 1 and a light chain variable region having the amino acid sequence of SEQ ID NO: 8;

(vi) a heavy chain variable region having the amino acid sequence of SEQ ID NO: 2 and a light chain variable region having the amino acid sequence of SEQ ID NO: 4;

(vii) a heavy chain variable region having the amino acid sequence of SEQ ID NO: 2 and a light chain variable region having the amino acid sequence of SEQ ID NO: 5;

(viii) a heavy chain variable region having the amino acid sequence of SEQ ID NO: 2 and a light chain variable region having the amino acid sequence of SEQ ID NO: 6;

(ix) a heavy chain variable region having the amino acid sequence of SEQ ID NO: 2 and a light chain variable region having the amino acid sequence of SEQ ID NO: 7;

(x) a heavy chain variable region having the amino acid sequence of SEQ ID NO: 2 and a light chain variable region having the amino acid sequence of SEQ ID NO: 8;

(xi) a heavy chain variable region having the amino acid sequence of SEQ ID NO: 3 and a light chain variable region having the amino acid sequence of SEQ ID NO: 4;

(xii) a heavy chain variable region having the amino acid sequence of SEQ ID NO: 3 and a light chain variable region having the amino acid sequence of SEQ ID NO: 5;

(xiii) a heavy chain variable region having the amino acid sequence of SEQ ID NO: 3 and a light chain variable region having the amino acid sequence of SEQ ID NO: 6;

(xiv) a heavy chain variable region having the amino acid sequence of SEQ ID NO: 3 and a light chain variable region having the amino acid sequence of SEQ ID NO: 7; or

(xv) a heavy chain variable region having the amino acid sequence of SEQ ID NO: 3 and a light chain variable region having the amino acid sequence of SEQ ID NO: 8.

In some embodiments AXL is human AXL.

The sequences of the antibody heavy chain variable regions and/or the light chain variable regions disclosed herein may be modified by, for example, insertions, substitutions and/or deletions to the extent that the humanized antibody maintains the ability to bind to AXL. The skilled person can ascertain the maintenance of this activity by performing the functional assays described herein, or known in the art.

Accordingly, in some embodiments the heavy chain variable region comprises no more than 20 insertions, substitutions and/or deletions, such as no more than 15, no more than 10, no more than 9, no more than 8, no more than 7, no more than 6, no more than 5, no more than 4, no more than 3, no more than 2, or no more than 1 insertion, substitution and/or deletion. In some embodiments the light chain variable region comprises no more than 20 insertions, substitutions and/or deletions, such as no more than 15, no more than 10, no more than 9, no more than 8, no more than 7, no more than 6, no more than 5, no more than 4, no more than 3, no more than 2, or no more than 1 insertion, substitution and/or deletion.

In some embodiments the humanized antibodies of the disclosure include antibodies comprising V_{H} and V_{L} domains with amino acid sequences that are identical to the sequences described herein.

Also disclosed herein are conjugates comprising the above described antibodies. Examples of antibody conjugates encompassed by the disclosure include conjugates of a drug, reporter, organic moiety, and/or binding moiety. Particularly preferred are antibody-drug conjugates comprising pyrrolobenzodiazepines (PBDs) having a labile C2 or N10 protecting group in the form of a linker to the humanized anti-AXL antibody.

### DETAILED DISCLOSURE

### Antibody Properties

### Antigen Binding

The antibody of the conjugates described herein is an antibody (Ab) which binds AXL. That is, the conjugates described herein are conjugates comprising antibodies which specifically bind to AXL.

As used herein, AXL refers to the Axl member of the TAM family of receptor tyrosine kinases. ‘Human Axl’ refers to the Axl member of the human TAM family of receptor tyrosine kinases. In some embodiments, the human Axl polypeptide corresponds to Genbank accession no. AAH32229, version no. AAH32229.1 GI:21619004, record update date: Mar. 6, 2012 01:18 PM (SEQ ID NO. 9). In one embodiment, the nucleic acid encoding the human Axl polypeptide corresponds to Genbank accession no. M76125, version no. M76125.1 G1:292869, record update date: Jun. 23, 2010 08:53 AM. ‘Murine Axl’ refers to the Axl member of the murine TAM family of receptor tyrosine kinases. In some embodiments, the murine Axl polypeptide corresponds to Genbank accession no. AAH46618, version no. AAH46618.1 GI:55777082, record update date: Mar. 6, 2012 01:36 PM (SEQ ID NO. 10). In one embodiment, the nucleic acid encoding the murine Axl polypeptide corresponds to Genbank accession no. NM_009465, version no. NM_009465.4 G1:300794836, record update date: Mar. 12, 2014 03:52 PM.

### Antibody Affinity

In some embodiments the humanized antibody binds human AXL with a dissociation constant (K_{D}) of at least 10⁻⁶ M, such as at least 5×10⁻⁷ M, at least 10⁻⁷ M, at least 5×10⁻⁸ M, at least 10⁻⁹ M, such as at least 5×10⁻¹⁰ M, at least 10⁻¹⁰ M, at least 5×10⁻¹¹ M, at least 10⁻¹¹ M, at least 5×10⁻¹² M, at least 10⁻¹² M, at least 5×10⁻¹³ M, at least 10⁻¹³ M, at least 5×10⁻¹⁴ M, at least 10⁻¹⁴ M, at least 5×10⁻¹⁵ M, or at least 10⁻¹⁵ M.

In one embodiment the humanized antibody competitively inhibits the in vivo and/or in vitro binding to human AXL of an antibody comprising a heavy chain variable region having the amino acid sequence of SEQ ID NO: 1 and a light chain variable region having the amino acid sequence of SEQ ID NO: 4. In one embodiment the humanized antibody competitively inhibits the in vivo and/or in vitro binding to human-AXL of the ‘mouse 1H12’ antibody. In some embodiments an equimolar dose of the humanised antibody competitively inhibits at least 20% of the binding by the ‘mouse 1H12’ antibody, such as at least 30%, at least 40%, at least 50%, at least 60%, at least 70%, at least 80%, or at least 90% of the binding. Percentage binding may be measured by, for example, a competitive ELISA assay where % inhibition of binding is calculated as [(1−absorbance of test sample)/(absorbance of negative control)].

In some embodiments the humanized antibody has a higher affinity for an Axl antigen (for example the Axl-Strep-His antigen described in Protocol 4) than an antibody comprising a VH domain having the sequence according to SEQ ID NO. 1, a VL domain having the sequence according to SEQ ID NO. 4, and a constant region derived from one or more human antibodies (for example, Ab1 described herein). In some embodiments the KD of the humanized antibody with the Axl antigen (for example the Axl-Strep-His antigen described in Protocol 4) will be no more than 0.9 of the KD of the antibody comprising a VH domain having the sequence according to SEQ ID NO. 1, a VL domain having the sequence according to SEQ ID NO. 4, and a constant region derived from one or more human antibodies, for example no more than 0.8, 0.7, 0.6, 0.5, 0.4, 0.3, 0.2, 0.1, 0.05, 0.01, or 0.001 of the KD of the antibody comprising a VH domain having the sequence according to SEQ ID NO. 1, a VL domain having the sequence according to SEQ ID NO. 4, and a constant region derived from one or more human antibodies.

In some embodiments the humanized antibody has a higher affinity for an Axl antigen (for example the Axl-Fc antigen described in Protocol 4) than an antibody comprising a VH domain having the sequence according to SEQ ID NO. 1, a VL domain having the sequence according to SEQ ID NO. 4, and a constant region derived from one or more human antibodies (for example, Ab1 described herein). In some embodiments the KD of the humanized antibody with the Axl antigen (for example the Axl-Fc antigen described in Protocol 4) will be no more than 0.9 of the KD of the antibody comprising a VH domain having the sequence according to SEQ ID NO. 1, a VL domain having the sequence according to SEQ ID NO. 4, and a constant region derived from one or more human antibodies, for example no more than 0.8, 0.7, 0.6, 0.5, 0.4, 0.3, 0.2, 0.1, 0.05, 0.01, or 0.001 of the KD of the antibody comprising a VH domain having the sequence according to SEQ ID NO. 1, a VL domain having the sequence according to SEQ ID NO. 4, and a constant region derived from one or more human antibodies.

### Antibody Isoelectric Point (pI)

A molecule carries no net charge when the pH of its surrounding equal the molecules pI. The net charge of a molecule affects the solubility of the molecule, with biological molecules such as proteins typically having minimum solubility in water or salt solutions at the pH that corresponds to their pI. Thus, proteins whose pI is 7.35-7.45 are at their minimum solubility in human blood, whose pH is typically in the range 7.35-7.45.

In some embodiments the humanized antibody of the disclosure has a pI greater than an antibody comprising a heavy chain variable region having the amino acid sequence of SEQ ID NO: 1 and a light chain variable region having the amino acid sequence of SEQ ID NO: 4. In some embodiments the humanized antibody of the disclosure has a pI greater than the mouse 1H12 antibody. In some embodiments the humanized antibody of the disclosure has a pI of at least 8.00, such as at least 8.05, at least 8.10, at least 8.15, at least 8.20, at least 8.30, at least 8.40, at least 8.50, at least 9, at least 9.5, at least 10, at least 10.5, or at least 11.

In some embodiments the humanized antibody of the disclosure has a pI less than an antibody comprising a heavy chain variable region having the amino acid sequence of SEQ ID NO: 1 and a light chain variable region having the amino acid sequence of SEQ ID NO: 4. In some embodiments the humanized antibody of the disclosure has a pI less than the mouse 1H12 antibody. In some embodiments the humanized antibody of the disclosure has a pI of no more than 7.0, such as no more than 6.5, no more than 6.0, no more than 5.5, no more than 5.0, no more than 4.5, or no more than 4.0.

### Antibody Immunogenicity

Preferably the humanized antibody of the disclosure has reduced immunogenicity in a human subject as compared to a non-humanized antibody of the same specificity (for example, a mouse antibody precursor prior to humanization. In one embodiment the humanized antibody has immunogenicity in a human subject lower than an otherwise identical antibody or antibody fragment comprising a heavy chain variable region having the amino acid sequence of SEQ ID NO: 1 and a light chain variable region having the amino acid sequence of SEQ ID NO: 4. In one embodiment the humanized antibody has immunogenicity in a human subject lower than the ‘mouse 1H12’ antibody.

Low or reduced immunogenicity can be characterized by the ability to treat patients for extended periods with measurable alleviation of symptoms and low and/or acceptable toxicity. Low or acceptable immunogenicity and/or high affinity, as well as other suitable properties, can contribute to the therapeutic results achieved. “Reduced immunogenicity” is defined herein as raising significant HAHA, HACA or HAMA responses in less 90%, such as less than 80%, less than 70%, less than 60%, less than 50%, less than 40%, less than 30%, less than 20%, less than 10% of the proportion of patients who show a significant HAHA, HACA or HAMA response when treated with the mouse 1H12 antibody.

The disclosure also provided the means produce the antibodies of the disclosure.

Accordingly, in another aspect the disclosure provides nucleic acid molecules encoding the humanised antibodies, along with nucleic acid molecules complementary to nucleic acid molecules encoding the humanised antibodies.

In another aspect, the disclosure provides a pharmaceutical composition comprising an antibody pf the disclosure, optionally further comprising a pharmaceutically acceptable carrier or excipient.

In another aspect the disclosure provides a vector, such as an expression vector, comprising a nucleic acid of the disclosure.

In another aspect, the disclosure provides host cells transfected with a vector of the disclosure. The host cells may be prokaryotic or eukaryotic. For example, the cells may be bacterial, fungal, insect, or mammalian (such as mouse, primate or human).

In another aspect the disclosure provides a method of making the antibodies by culturing the host cells of the disclosure.

The disclosure provides methods relating to the identification of subjects particularly suitable for treatment with the antibodies or pharmaceutical composition of the disclosure. Also provided are methods for determining the optimum timing and dosage of administration of the antibodies of the disclosure to a subject. In some embodiments the subject has a proliferative disease, such as cancer. In some embodiments the subject has an autoimmune disease. Preferably, administration of the treatment inhibits or reduces one or more aspects of the disease, for example reduces tumour volume, or reduces the level of one or more biomarkers of tumour progression, such as AXL, Akt3, or GAS6. In some embodiments the level of the biomarker is reduced to no more than 90% of the level immediately before treatment, such as no more than 80%, no more than 70%, no more than 60%, no more than 50%, no more than 40%, no more than 30%, no more than 20%, no more than 10%, or no more than 5% of the level immediately before treatment.

In one aspect the disclosure provides a method of selecting a subject for treatment with the antibody or pharmaceutical composition of the disclosure, the method comprising assessing the level of one or more biomarkers associated with disease pathology, wherein subjects having the one or more biomarker, or subjects having a level of the one or more biomarkers which exceeds a threshold level, are selected for treatment. In some embodiments the biomarker is AXL, Akt3, or GAS6. In some embodiments the threshold is at least 10% higher than the upper boundary of the normal clinical range, such as at least 20% higher, at least 30% higher, at least 40% higher, at least 50% higher, at least 100% higher, or at least 200% higher.

In another aspect the disclosure provides a method of timing the administration of treatment of a subject with the antibody or pharmaceutical composition of the disclosure, the method comprising assessing the level of one or more biomarkers associated with disease pathology, wherein the treatment is administered when the subject has the one or more biomarker, or the subject has a level of one or more biomarkers which exceeds a threshold level. In some embodiments the biomarker is AXL, Akt3, or GAS6. In some embodiments the threshold is at least 10% higher than the upper boundary of the normal clinical range, such as at least 20% higher, at least 30% higher, at least 40% higher, at least 50% higher, at least 100% higher, or at least 200% higher.

In another aspect the disclosure provides a method of determining the optimum dosage of the antibody or pharmaceutical composition of the disclosure for administration to a subject, the method comprising assessing the level of one or more biomarkers associated with disease pathology, wherein subjects having the one or more biomarker, or subjects having a level of the one or more biomarkers which exceeds the threshold level, are selected for a particular dosage level. In some embodiments the biomarker is AXL, Akt3, or GAS6. In some embodiments the threshold is at least 10% higher than the upper boundary of the normal clinical range, such as at least 20% higher, at least 30% higher, at least 40% higher, at least 50% higher, at least 100% higher, or at least 200% higher.

In some embodiments the level of one or more biomarkers is assessed in a sample of blood, urine, other body fluid, or tissue. Level of one or more biomarkers samples can be assessed by immunoassay, proteomic assay, nucleic acid hybridization or amplification assays, immunohistochemistry, or in situ hybridization assays.

### Conjugates

The humanised antibody of the disclosure may be conjugated to a functional moiety. The conjugation may be via, for example, chemical coupling, genetic fusion, non-covalent association or otherwise. In preferred embodiments the antibody and functional moiety are conjugated via covalent attachment. Conjugation between the antibody and functional moiety may be direct or indirect (for example, through linker sequences). One example of indirect linkage is then the functional moiety is a radionucleotide chelated by a macrocyclic chelators such as 1,4,7,10-tetraazacyclododecane-N, N′,N″,N″tetraacetic acid (DOTA) which can be attached to the antibody via a linker molecule.

Examples of functional moieties include an amino acid, a peptide, a protein, a polysaccharide, a nucleoside, a nucleotide, an oligonucleotide, a nucleic acid, a drug, a hormone, a lipid, a lipid assembly, a synthetic polymer, a polymeric microparticle, a biological cell, a virus, a reporter (such as a fluorophore, a chromophore, or a dye), a toxin, a hapten, an enzyme, a binding member (such as an antibody, or an antibody fragment), a radioisotope, solid matrixes, semisolid matrixes and combinations thereof, or an organic moiety. In preferred embodiments the functional moiety is a drug moiety.

### Antibody-Drug Conjugates

Antibody therapy has been established for the targeted treatment of patients with cancer, immunological and angiogenic disorders (Carter, P. (2006) Nature Reviews Immunology 6:343-357). The use of antibody-drug conjugates (ADC), i.e. immunoconjugates, for the local delivery of cytotoxic or cytostatic agents, i.e. drugs to kill or inhibit tumor cells in the treatment of cancer, targets delivery of the drug moiety to tumors, and intracellular accumulation therein, whereas systemic administration of these unconjugated drug agents may result in unacceptable levels of toxicity to normal cells (Xie et al (2006) Expert. Opin. Biol. Ther. 6(3):281-291; Kovtun et al (2006) Cancer Res. 66(6):3214-3121; Law et al (2006) Cancer Res. 66(4):2328-2337; Wu et al (2005) Nature Biotech. 23(9):1137-1145; Lambert J. (2005) Current Opin. in Pharmacol. 5:543-549; Hamann P. (2005) Expert Opin. Ther. Patents 15(9):1087-1103; Payne, G. (2003) Cancer Cell 3:207-212; Trail et al (2003) Cancer Immunol. Immunother. 52:328-337; Syrigos and Epenetos (1999) Anticancer Research 19:605-614).

Maximal efficacy with minimal toxicity is sought thereby. Efforts to design and refine ADC have focused on the selectivity of monoclonal antibodies (mAbs) as well as drug mechanism of action, drug-linking, drug/antibody ratio (loading), and drug-releasing properties (Junutula, et al., 2008b Nature Biotech., 26(8):925-932; Dornan et al (2009) Blood 114(13):2721-2729; U.S. Pat. No. 7,521,541; U.S. Pat. No. 7,723,485; WO2009/052249; McDonagh (2006) Protein Eng. Design & Sel. 19(7): 299-307; Doronina et al (2006) Bioconj. Chem. 17:114-124; Erickson et al (2006) Cancer Res. 66(8):1-8; Sanderson et al (2005) Clin. Cancer Res. 11:843-852; Jeffrey et al (2005) J. Med. Chem. 48:1344-1358; Hamblett et al (2004) Clin. Cancer Res. 10:7063-7070). Drug moieties may impart their cytotoxic and cytostatic effects by mechanisms including tubulin binding, DNA binding, proteasome and/or topoisomerase inhibition. Some cytotoxic drugs tend to be inactive or less active when conjugated to large antibodies or protein receptor ligands. A preferred class of drug is pyrrolobenzodiazepines (PBDs).

Described herein are conjugates comprising a pyrrolobenzodiazepine (PBD) drug moiety with a labile C2 or N10 protecting group and an antibody which binds AXL. Also described herein are conjugates comprising an antibody fragment as described herein, along with pharmaceutical compositions comprising the conjugates. Example antibodies or antibody fragment include scFv-Fc fusions and minibodies. Methods of preparing the conjugates and using the conjugates are disclosed, along with methods of using the conjugates to treat a number of diseases.

Pyrrolobenzodiazeines (PBDs)

The conjugates described herein comprise a PBD drug moiety. Some pyrrolobenzodiazepines (PBDs) have the ability to recognise and bond to specific sequences of DNA; the preferred sequence is PuGPu. The first PBD antitumour antibiotic, anthramycin, was discovered in 1965 (Leimgruber, et al., J. Am. Chem. Soc., 87, 5793-5795 (1965); Leimgruber, et al., J. Am. Chem. Soc., 87, 5791-5793 (1965)). Since then, a number of naturally occurring PBDs have been reported, and over 10 synthetic routes have been developed to a variety of analogues (Thurston, et al., Chem. Rev. 1994, 433-465 (1994); Antonow, D. and Thurston, D. E., Chem. Rev. 2011 111 (4), 2815-2864). Family members include abbeymycin (Hochlowski, et al., J. Antibiotics, 40, 145-148 (1987)), chicamycin (Konishi, et al., J. Antibiotics, 37, 200-206 (1984)), DC-81 (Japanese Patent 58-180 487; Thurston, et al., Chem. Brit., 26, 767-772 (1990); Bose, et al., Tetrahedron, 48, 751-758 (1992)), mazethramycin (Kuminoto, et al., J. Antibiotics, 33, 665-667 (1980)), neothramycins A and B (Takeuchi, et al., J. Antibiotics, 29, 93-96 (1976)), porothramycin (Tsunakawa, et al., J. Antibiotics, 41, 1366-1373 (1988)), prothracarcin (Shimizu, et al, J. Antibiotics, 29, 2492-2503 (1982); Langley and Thurston, J. Org. Chem., 52, 91-97 (1987)), sibanomicin (DC-102)(Hara, et al., J. Antibiotics, 41, 702-704 (1988); Itoh, et al., J. Antibiotics, 41, 1281-1284 (1988)), sibiromycin (Leber, et al., J. Am. Chem. Soc., 110, 2992-2993 (1988)) and tomamycin (Arima, et al., J. Antibiotics, 25, 437-444 (1972)). PBDs are of the general structure:

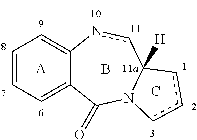

They differ in the number, type and position of substituents, in both their aromatic A rings and pyrrolo C rings, and in the degree of saturation of the C ring. In the B-ring there is either an imine (N═C), a carbinolamine (NH—CH(OH)), or a carbinolamine methyl ether (NH—CH(OMe)) at the N10-C11 position which is the electrophilic centre responsible for alkylating DNA. All of the known natural products have an (S)-configuration at the chiral C11a position which provides them with a right-handed twist when viewed from the C ring towards the A ring. This gives them the appropriate three-dimensional shape for isohelicity with the minor groove of B-form DNA, leading to a snug fit at the binding site (Kohn, In Antibiotics III. Springer-Verlag, New York, pp. 3-11 (1975); Hurley and Needham-VanDevanter, Acc. Chem. Res., 19, 230-237 (1986)). Their ability to form an adduct in the minor groove, enables them to interfere with DNA processing, hence their use as antitumour agents.

One pyrrolobenzodiazepine compound is described by Gregson et al. (Chem. Commun. 1999, 797-798) as compound 1, and by Gregson et al. (J. Med. Chem. 2001, 44, 1161-1174) as compound 4a. This compound, also known as SG2000, is shown below:

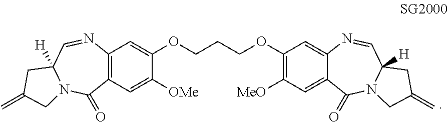

WO 2007/085930 describes the preparation of dimer PBD compounds having linker groups for connection to a cell binding agent, such as an antibody. The linker is present in the bridge linking the monomer PBD units of the dimer.

WO 2011/130613 and WO 2011/130616 describe dimer PBD compounds having linker groups for connection to a cell binding agent, such as an antibody. The linker in these compounds is attached to the PBD core via the C2 position, and are generally cleaved by action of an enzyme on the linker group. In WO 2011/130598, the linker in these compounds is attached to one of the available N10 positions on the PBD core, and are generally cleaved by action of an enzyme on the linker group.

Conjugates Comprising PBD Drug Moieties

The present inventors have found that conjugates where the Drug unit (D^{L}) is conjugated to an antibody which binds AXL, as described herein, have unexpected and advantageous such as improved solubility.

Accordingly, in one aspect the disclosure provides a conjugate of formula L-(DL)p, where DL is of formula I or II:

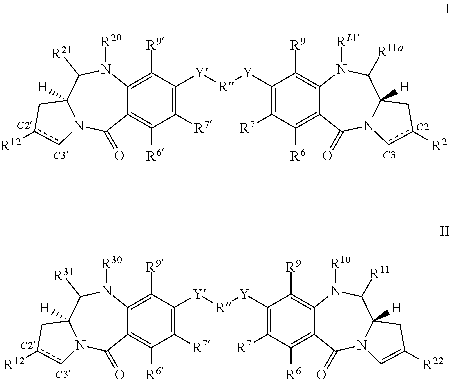

wherein:

L is an isolated humanized antibody that binds to AXL (Ab) as defined above; when there is a double bond present between C2′ and C3′, R¹² is selected from the group consisting of:

(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;

(ib) C₁₋₅ saturated aliphatic alkyl;

(ic) C₃₋₆ saturated cycloalkyl;

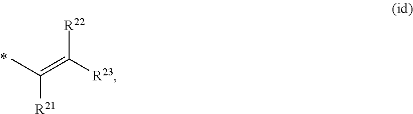

wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5;

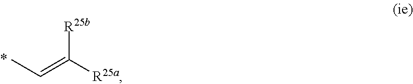

wherein one of R^{25a} and R^{25b} is H and the other is selected from: phenyl, which phenyl is optionally substituted by a group selected from halo, methyl, methoxy; pyridyl; and thiophenyl; and

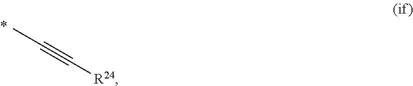

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2′ and C3′,

R¹² is

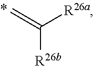

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;

R⁶ and R⁹ are independently selected from H, R, OH, OR, SH, SR, NH₂, NHR, NRR′, nitro, Me₃Sn and halo;

where R and R′ are independently selected from optionally substituted C₁₋₁₂ alkyl, C₃₋₂₀ heterocyclyl and C₅₋₂₀ aryl groups;

R⁷ is selected from H, R, OH, OR, SH, SR, NH₂, NHR, NHRR′, nitro, Me₃Sn and halo;

R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;

Y and Y′ are selected from O, S, or NH;

R^{6′}, R^{7′}, R^{9′} are selected from the same groups as R⁶, R⁷ and R⁹ respectively;

[Formula I]

R^{L1′} is a linker for connection to the antibody (Ab);

R^{11a} is selected from OH, OR^{A}, where R^{A} is C₁₋₄ alkyl, and SO_{z}M, where z is 2 or 3 and M is a monovalent pharmaceutically acceptable cation;

R²⁰ and R²¹ either together form a double bond between the nitrogen and carbon atoms to which they are bound or;

R²⁰ is selected from H and R^{C}, where R^{C} is a capping group;

R²¹ is selected from OH, OR^{A} and SO_{z}M;

when there is a double bond present between C2 and C3, R² is selected from the group consisting of:

(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;

(ib) C₁₋₅ saturated aliphatic alkyl;

(ic) C₃₋₆ saturated cycloalkyl;

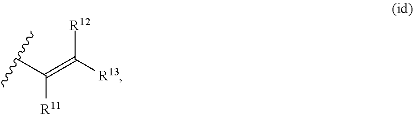

wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;

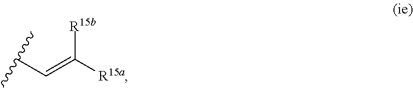

wherein one of R^{15a} and R^{15b} is H and the other is selected from: phenyl, which phenyl is optionally substituted by a group selected from halo, methyl, methoxy; pyridyl; and thiophenyl; and

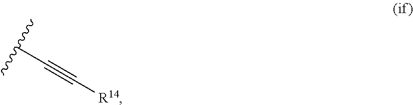

where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2 and C3,

R¹² is

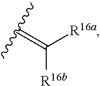

where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;

[Formula II]

R²² is of formula IIIa, formula IIIb or formula IIIc:

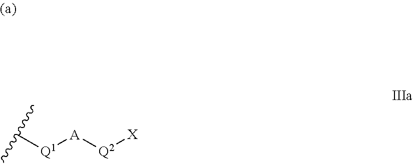

where A is a C₅₋₇ aryl group, and either

(i) Q¹ is a single bond, and Q² is selected from a single bond and —Z—(CH₂)ₙ—, where Z is selected from a single bond, O, S and NH and n is from 1 to 3; or

(ii) Q¹ is —CH═CH—, and Q² is a single bond;

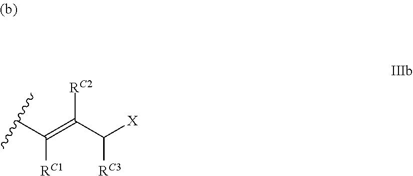

where;

R^{C1}, R^{C2} and R^{C3} are independently selected from H and unsubstituted C₁₋₂ alkyl;

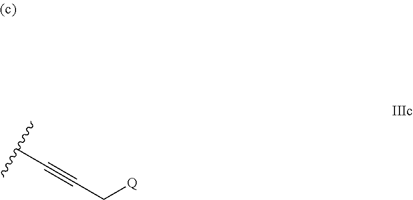

where Q is selected from O—R^{L2′}, S—R^{L2′} and NR^{N}—R^{L2′}, and R^{N} is selected from H, methyl and ethyl

X is selected from the group comprising: O—R^{L2′}, S—R^{L2′}, CO₂—R^{L2′}, CO—R^{L2′}, NH—C(═O)—R^{L2′}, NHNH—R^{L2′}, CONHNH—R^{L2′},

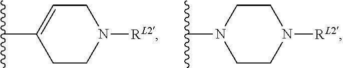

NR^{N}R^{L2′}, wherein R^{N} is selected from the group comprising H and C₁₋₄ alkyl;

R^{L2′} is a linker for connection to the antibody (Ab);

R¹⁰ and R¹¹ either together form a double bond between the nitrogen and carbon atoms to which they are bound or;

R¹⁰ is H and R¹¹ is selected from OH, OR^{A} and SO_{z}M;

R³⁰ and R³¹ either together form a double bond between the nitrogen and carbon atoms to which they are bound or;

R³⁰ is H and R³¹ is selected from OH, OR^{A} and SO_{z}M.

In some embodiments, it may be preferred that the conjugate is selected from a conjugate of formula ConjA, ConjB, ConjC, ConjD, ConjE, ConjF, ConjG and ConjH:

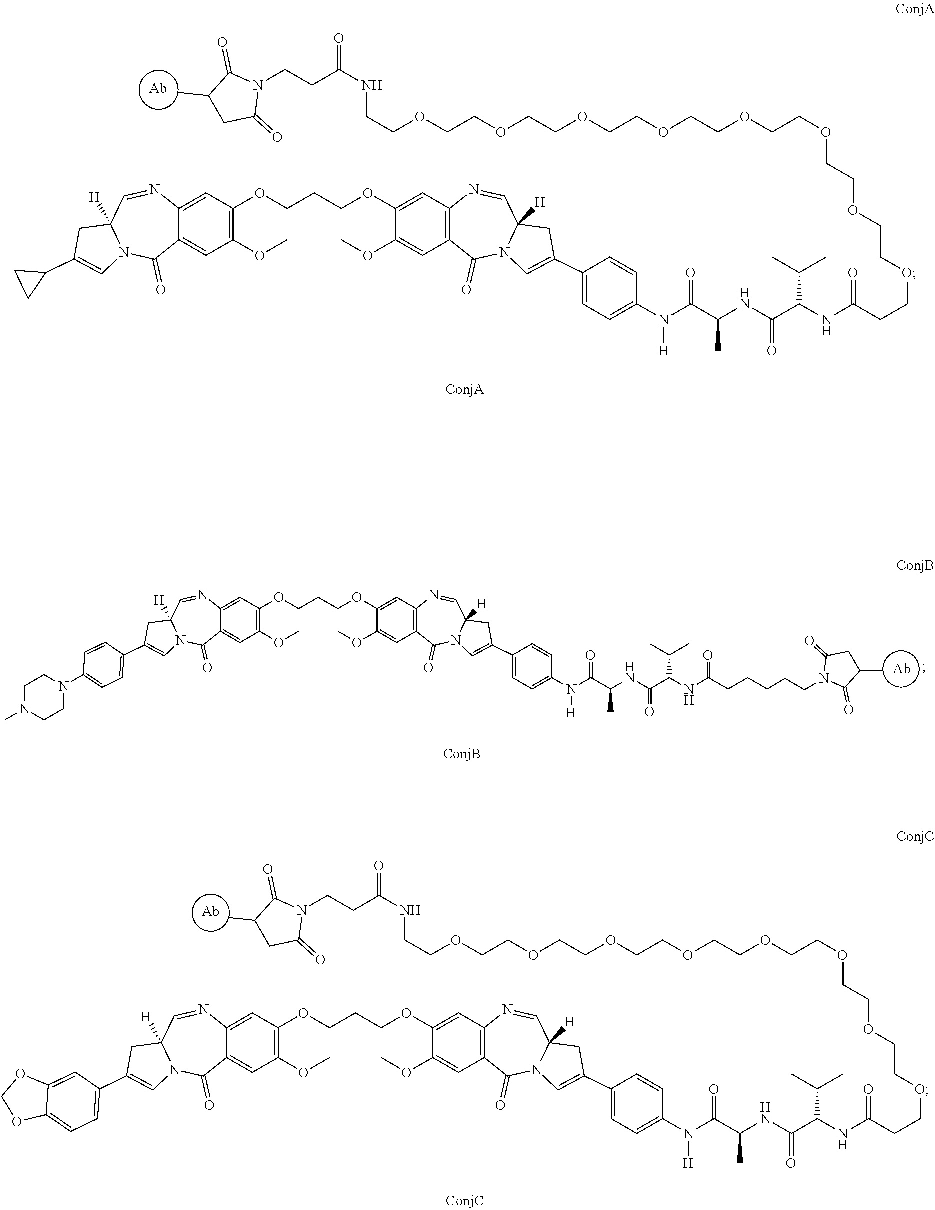

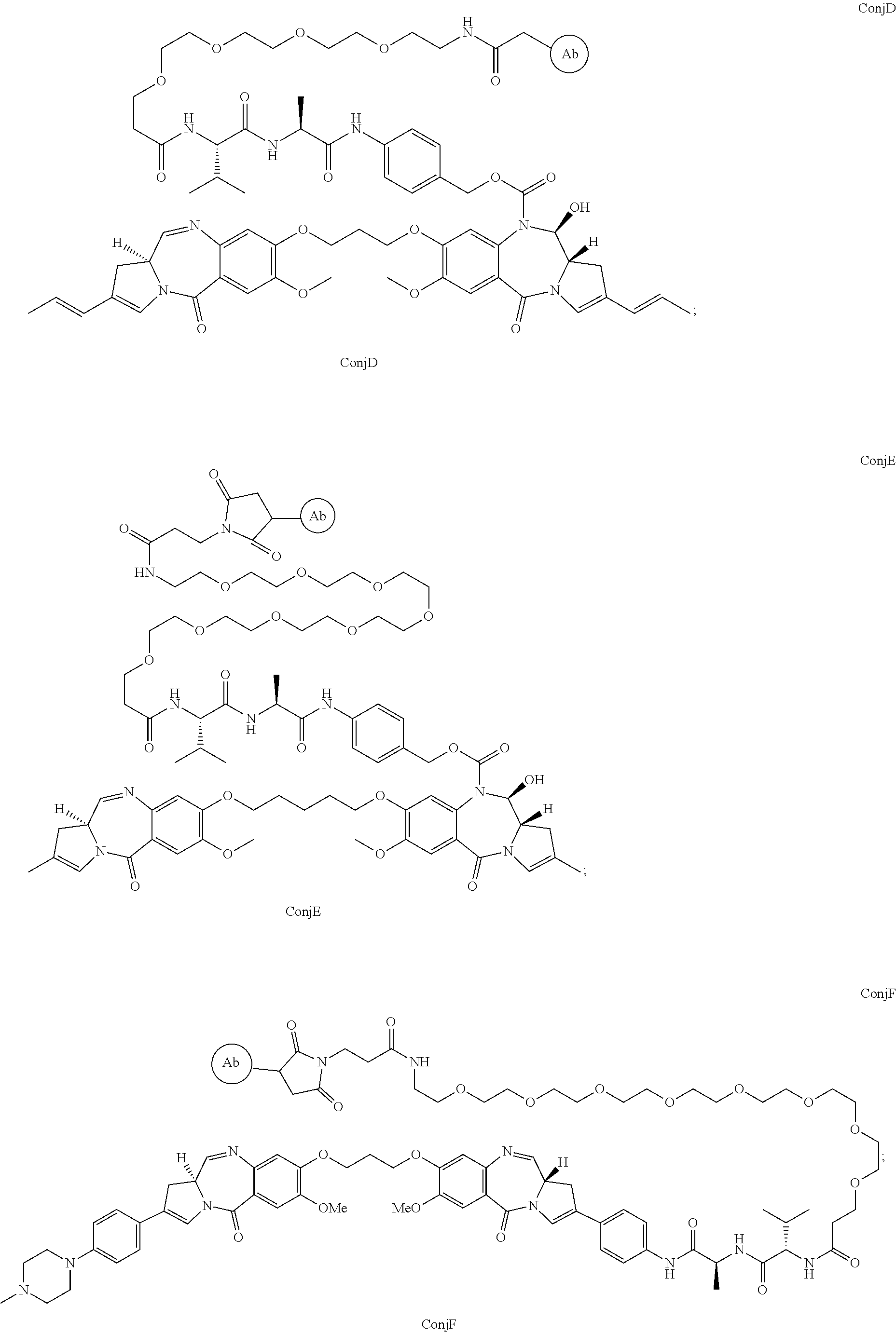

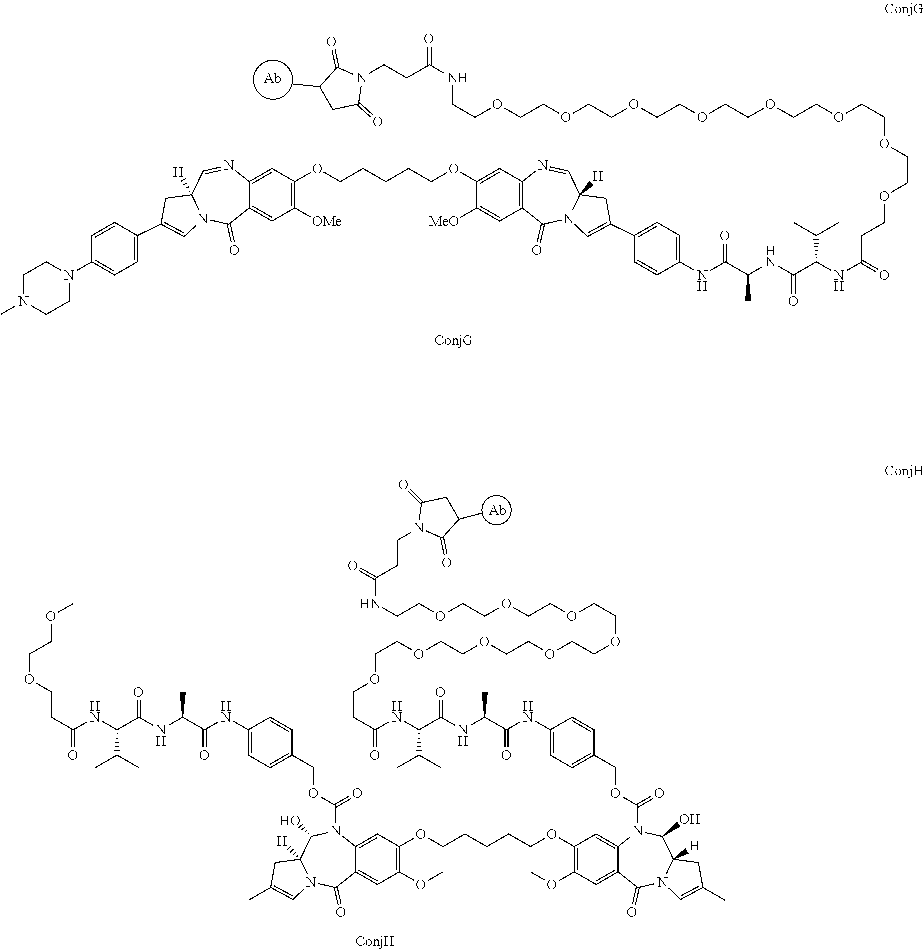

The link to the moiety shown is via a free S (active thiol) of a cysteine residue on the cell binding agent.

The subscript p in the formula I is an integer of from 1 to 20. Accordingly, the Conjugates comprise an antibody (Ab) as defined herein covalently linked to at least one Drug unit by a Linker unit. The Ligand unit, described more fully below, is a targeting agent that binds to a target moiety. Accordingly, also described herein are methods for the treatment of, for example, various cancers and autoimmune disease. The drug loading is represented by p, the number of drug molecules per antibody. Drug loading may range from 1 to 20 Drug units (D^{L}) per antibody. For compositions, p represents the average drug loading of the Conjugates in the composition, and p ranges from 1 to 20.

A second aspect of the disclosure provides a method of making a conjugate according to the first aspect of the disclosure comprising conjugating a compound of formula I^{L} or II^{L}:

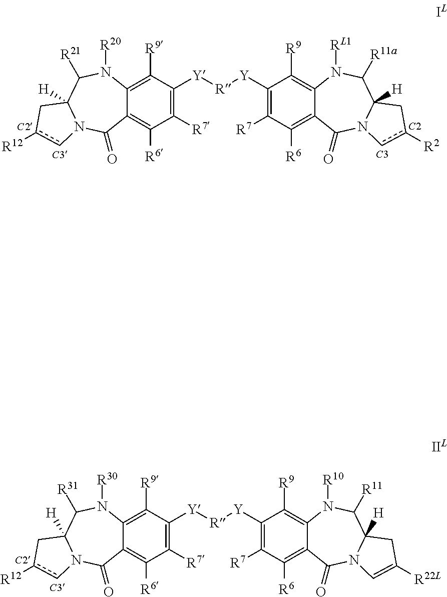

to the antibody (Ab) as defined below, wherein:

R^{L1} is a linker suitable for conjugation to the antibody (Ab);

R^{22L} is of formula IIIa^{L}, formula IIIb^{L} or formula s IIIc^{L}:

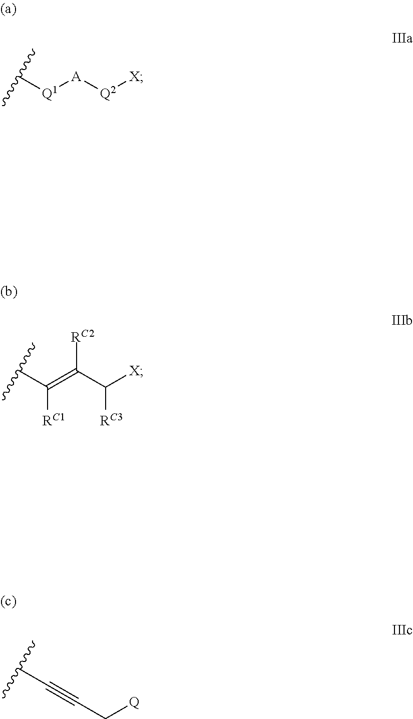

where Q^{L} is selected from O—R^{L2}, S—R^{L2} and NR^{N}—R^{L2}, and R^{N} is selected from H, methyl and ethyl

X^{L} is selected from the group comprising: O—R^{L2}, S—R^{L2}, CO₂—R^{L2}, CO—R^{L2}, N═C═O—R^{L2} NHNH—R^{L2}, CONHNH—R^{L2},

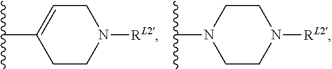

NR^{N}R^{L}, wherein R^{N} is selected from the group comprising H and C1-4 alkyl;

R^{L2} is a linker suitable for conjugation to the antibody (Ab);

and all the remaining groups are as defined in the first aspect.

Thus it may be preferred in the second aspect, that the disclosure provides a method of making a conjugate selected from the group consisting of ConjA, ConjB, ConjC, ConjD, ConjE, ConjF, ConjG and ConjH comprising conjugating a compound which is selected respectively from

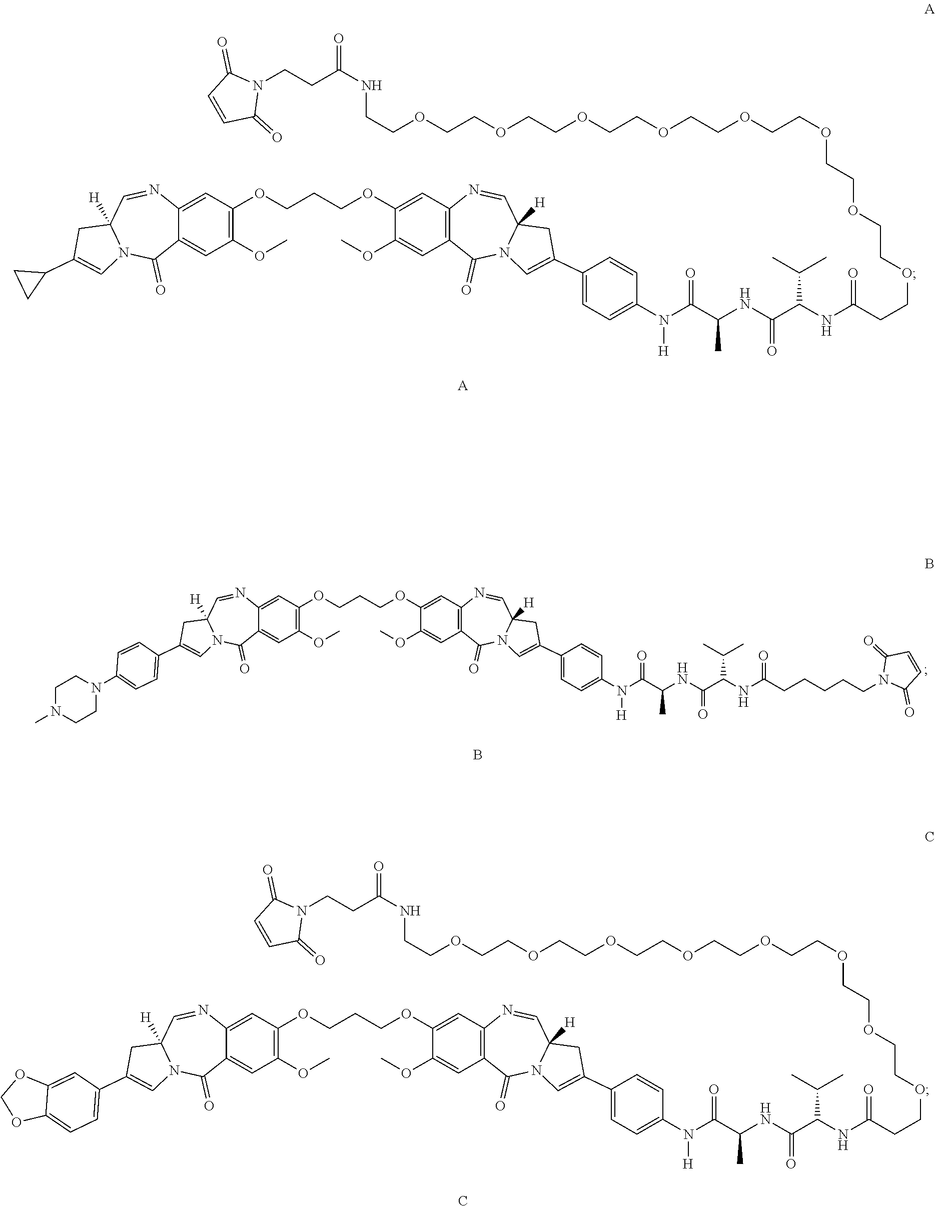

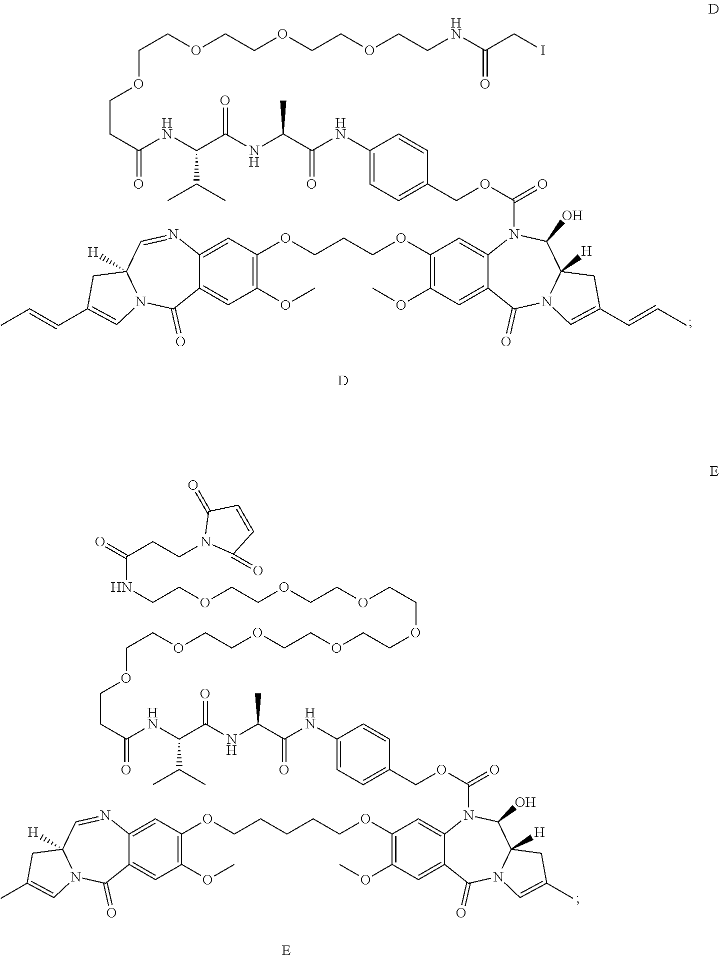

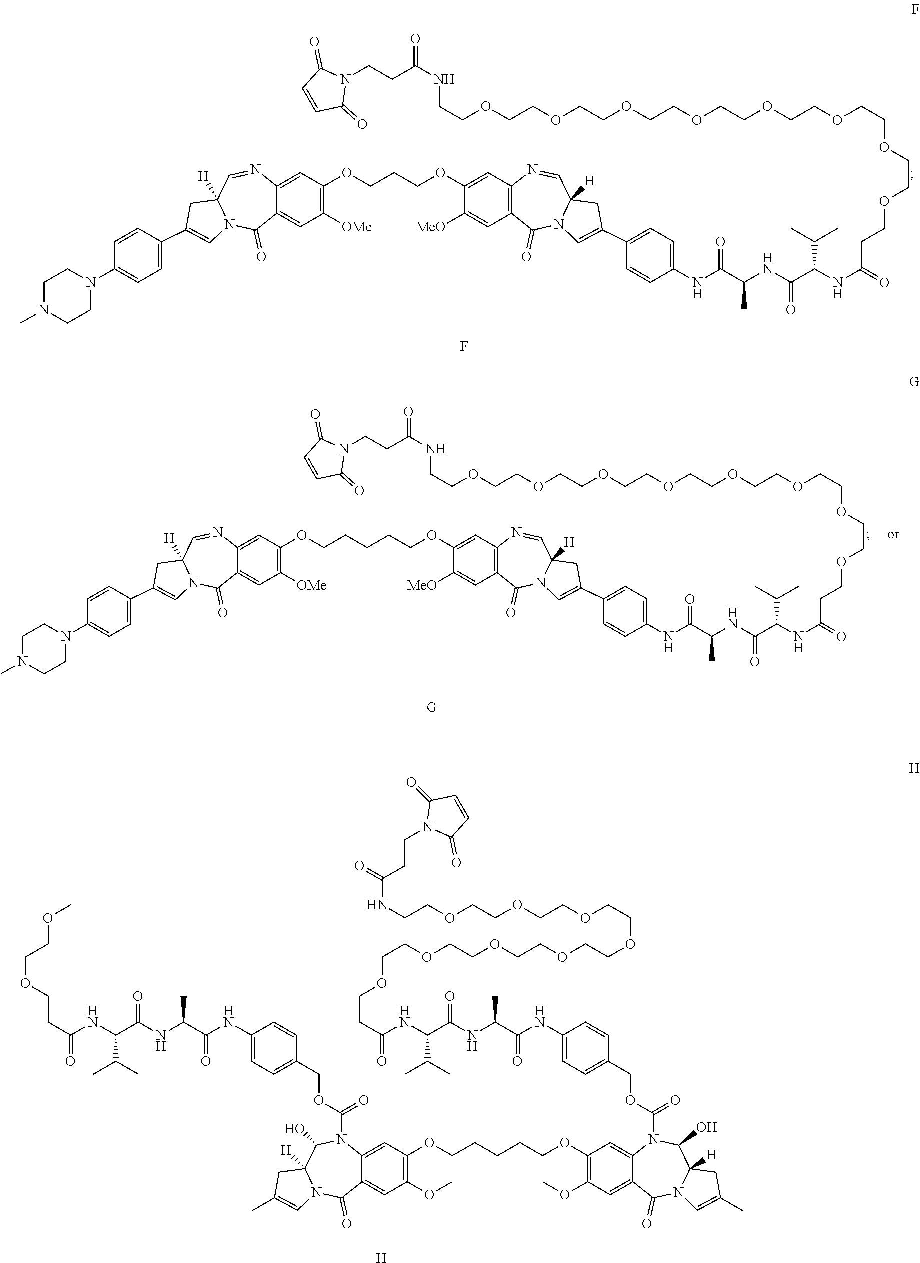

with an antibody as defined below.

Compounds A to E are disclosed in WO 2014/057073 and WO 2014/057074.

WO 2011/130613 discloses compound 51:

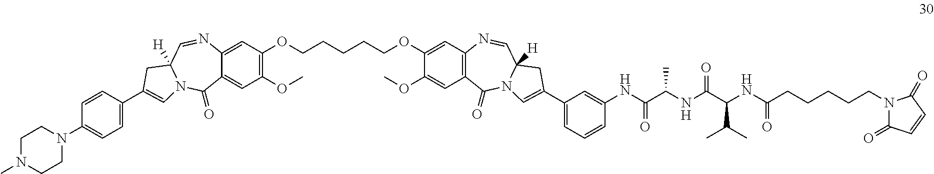 [SMILES: COc1cc2c(cc1OCCCCCOc1cc3c(cc1OC)C(=O)N1C=C(c4cccc(NC(=O)[C@H](C)NC(=O)[C@@H](NC(=O)CCCCCN5C(=O)C=CC5=O)C(C)C)c4)C[C@H]1C=N3)N=C[C@@H]1CC(c3ccc(N4CCN(C)CC4)cc3)=CN1C2=O]

WO 2013/041606 discloses Compound F (see compound 13e in WO 2013/041606). Compound F differs from compound 30 by only having a (CH₂)₃ tether between the PBD moieties, instead of a (CH₂)₅ tether, which reduces the lipophilicity of the released PBD dimer. The linking group in compounds F and G is attached to the C2-phenyl group in the para rather than meta position.

Compound H has a cleavable protecting group on the second imine group which avoids cross-reactions during its synthesis and in the final product avoids the formation of carbinolamine and carbinolamine methyl ethers. This protection also avoids the presence of an reactive imine group in the molecule.

Compounds A, B, C, D, E, F, G and H have two sp² centres in each C-ring, which may allow for stronger binding in the minor groove of DNA, than for compounds with only one sp² centre in each C-ring.

The drug linkers disclosed in WO 2010/043880, WO 2011/130613, WO 2011/130598, WO 2013/041606 and WO 2011/130616 may be used in the present disclosure, and are incorporated herein by reference. The drug linkers described herein may be synthesised as described in these disclosures.

Delivery of PBD Compounds

The present disclosure is suitable for use in providing a PBD compound to a preferred site in a subject. The conjugate may allow the release of an active PBD compound that does not retain any part of the linker. In such as case there is no stub present that could affect the reactivity of the PBD compound.

ConjA would release the compound RelA:

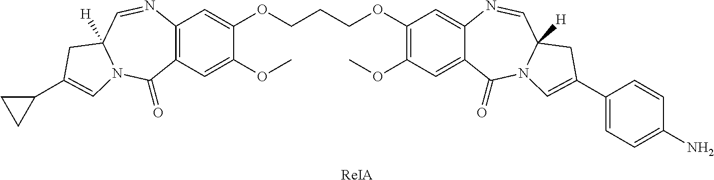 [SMILES: COc1cc2c(cc1OCCCOc1cc3c(cc1OC)C(=O)N1C=C(C4CC4)C[C@H]1C=N3)N=C[C@@H]1CC(c3ccc(N)cc3)=CN1C2=O ; "ReIA"]

ConjB and ConjF would release the compound RelB:

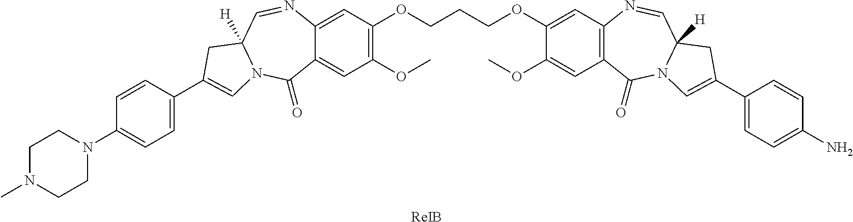 [SMILES: COc1cc2c(cc1OCCCOc1cc3c(cc1OC)C(=O)N1C=C(c4ccc(N5CCN(C)CC5)cc4)C[C@H]1C=N3)N=C[C@@H]1CC(c3ccc(N)cc3)=CN1C2=O ; "ReIB"]

ConjC would release the compound RelC:

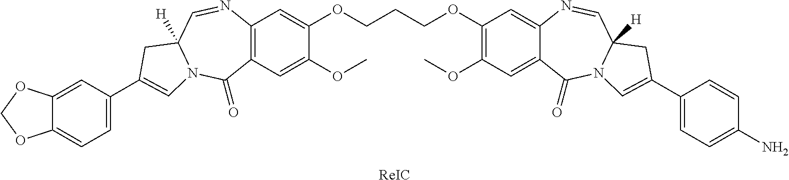 [SMILES: COc1cc2c(cc1OCCCOc1cc3c(cc1OC)C(=O)N1C=C(c4ccc5c(c4)OCO5)C[C@H]1C=N3)N=C[C@@H]1CC(c3ccc(N)cc3)=CN1C2=O ; "ReIC"]

ConjD would release the compound RelD:

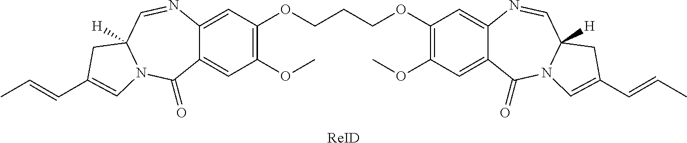 [SMILES: C/C=C/C1=CN2C(=O)c3cc(OC)c(OCCCOc4cc5c(cc4OC)C(=O)N4C=C(/C=C/C)C[C@H]4C=N5)cc3N=C[C@@H]2C1 ; "ReID"]

ConjE and ConjH would release the compound RelE:

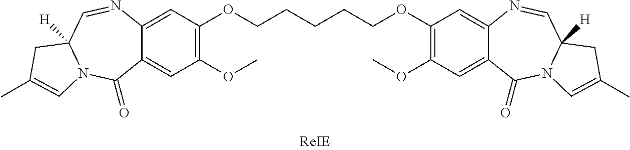

and ConjG would release the compound RelG:

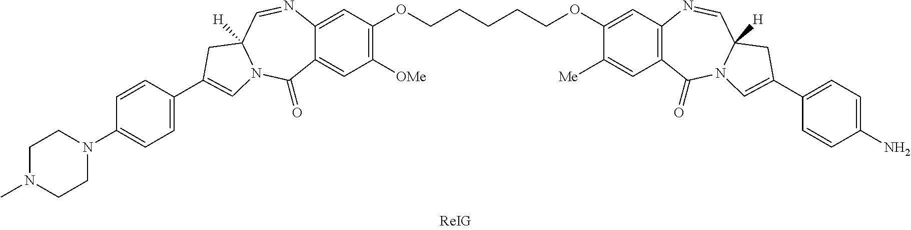 [SMILES: COc1cc2c(cc1OCCCCCOc1cc3c(cc1C)C(=O)N1C=C(c4ccc(N)cc4)C[C@H]1C=N3)N=C[C@@H]1CC(c3ccc(N4CCN(C)CC4)cc3)=CN1C2=O]

The specified link between the PBD dimer and the antibody, in the present disclosure is preferably stable extracellularly. Before transport or delivery into a cell, the antibody-drug conjugate (ADC) is preferably stable and remains intact, i.e. the antibody remains linked to the drug moiety. The linkers are stable outside the target cell and may be cleaved at some efficacious rate inside the cell. An effective linker will: (i) maintain the specific binding properties of the antibody; (ii) allow specific intracellular delivery of the conjugate or drug moiety; (iii) remain stable and intact, i.e. not cleaved, until the conjugate has been delivered or transported to its targeted site; and (iv) maintain a cytotoxic, cell-killing effect or a cytostatic effect of the PBD drug moiety. Stability of the ADC may be measured by standard analytical techniques such as in vitro cytotoxicity, mass spectroscopy, HPLC, and the separation/analysis technique LC/MS.

Delivery of the compounds of formulae RelA, RelB, RelC, RelD, RelE or RelG is achieved at the desired activation site of the conjugates of formulae ConjA, ConjB, ConjC, ConjD, ConjE, ConhF, ConjG or ConjH by the action of an enzyme, such as cathepsin, on the linking group, and in particular on the valine-alanine dipeptide moiety.

### Definitions

### Antibody

The term “antibody” as used encompasses any molecule comprising an antibody antigen-binding site (as, for example, formed by a paired VH domain and a VL domain). Thus, for example, the term “antibody” encompasses monoclonal antibodies (including intact monoclonal antibodies), polyclonal antibodies, multispecific antibodies formed from at least two different epitope binding fragments (e.g., bispecific antibodies), human antibodies, humanized antibodies, camelised antibodies, chimeric antibodies, single-chain antibodies (such as scFv fusions with CH3), antibody fragments that exhibit the desired biological activity (e.g. the antigen binding portion; for example minibodies), and anti-idiotypic (anti-Id) antibodies, intrabodies, and epitope-binding fragments of any of the above, so long as they exhibit the desired biological activity, for example, the ability to bind the cognate antigen. Antibodies may be murine, human, humanized, chimeric, or derived from other species. In one embodiment the antibody is a single-chain Fv antibody fused to a CH3 domain (scFv-CH3). In one embodiment the antibody is a single-chain Fv antibody fused to a Fc region (scFv-Fc). In one embodiment the antibody is a minibody.

An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes an intact immunoglobulin molecule or an immunologically active portion of a intact immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease.

In particular, antibodies include immunoglobulin molecules and immunologically active fragments of immunoglobulin molecules, i.e., molecules that contain at least one antigen binding site. The antibody can be of any isotype (e.g. IgG, IgE, IgM, IgD, and IgA), class (e.g. IgG1, IgG2, IgG3, IgG4, IgA1 and IgA2) or subclass, or allotype (e.g. human G1m1, G1m2, G1m3, non-G1m1 [that, is any allotype other than G1m1], G1m17, G2m23, G3m21, G3m28, G3m11, G3m5, G3m13, G3m14, G3m10, G3m15, G3m16, G3m6, G3m24, G3m26, G3m27, A2 ml, A2m2, Km1, Km2 and Km3) of antibody molecule. The immunoglobulins can be derived from any species, including human, murine, or rabbit origin.

An “intact antibody” herein is one comprising VL and VH domains, as well as a light chain constant domain (CL) and heavy chain constant domains, CH1, CH2 and CH3. The constant domains may be native sequence constant domains (e.g. human native sequence constant domains) or amino acid sequence variant thereof. The intact antibody may have one or more “effector functions” which refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors such as B cell receptor and BCR.

In preferred embodiments the antibody is an intact IgG antibody. That is an antibody comprising two light chains, each having a variable and constant domain, and two heavy chains, each having one variable domain and three constant domains.

### Humanized

As used herein “humanized” antibodies include any combination of the herein described Anti-AXL antibodies. In these antibodies the mouse framework residues from the murine 1H12 antibody have been largely replaced with the corresponding residues from human immunoglobulins. As many of the human amino acid residues as possible are retained, but critical human residues may be modified as necessary to support the antigen binding site formed by the CDRs and recapitulate the antigen binding potency of the original mouse antibody. Such changes or variations optionally and preferably retain or reduce the immunogenicity in humans or other primate species relative to non-modified antibodies.

It is pointed out that a humanized antibody can be produced by a non-human animal or prokaryotic or eukaryotic cell that is capable of expressing functionally rearranged human immunoglobulin (e.g., heavy chain and/or light chain) genes. Further, when the antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

For example, in some embodiments the humanised antibody of the disclosure are produced by a method comprising he step of grafting the CDRs of the mouse 1H12 antibody into human FW regions such as AB021508, AB063892, AF233253, and AJ399878. In some embodiments the method of producing the humanised antibodies of the invention further comprises the step of back-mutating mismatches at vernier and 5 Å CDR envelope residues. In other embodiments the method of producing the humanised antibodies of the invention further comprises the step of back-mutating mismatched vernier residues only.

The human constant region of the humanized antibody can be of any class (IgG, IgA, IgM, IgE, IgD, etc.) or isotype and can comprise a kappa or lambda light chain. In one embodiment, the human constant region comprises an IgG heavy chain or defined fragment, for example, at least one of isotypes, IgG1, IgG2, IgG3 or IgG4. In another embodiment, the humanized antibody comprises an IgG1 heavy chain and a IgG1 K light chain. The isolated humanized antibodies described herein comprise antibody amino acid sequences disclosed herein encoded by any suitable polynucleotide.

Sequence Modifications

The sequences of the antibody heavy chain variable regions and/or the light chain variable regions disclosed herein may be modified by substitution, insertion or deletion. Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Preferred conservative substitutions are those wherein one amino acid is substituted for another within the groups of amino acids indicated herein below:
Amino acids having polar side chains (Asp, Glu, Lys, Arg, His, Asn, Gin, Ser, Thr, Tyr, and Cys)
Amino acids having non-polar side chains (Gly, Ala, Val, Leu, Ile, Phe, Trp, Pro, and Met)
Amino acids having aliphatic side chains (Gly, Ala Val, Leu, Ile)
Amino acids having cyclic side chains (Phe, Tyr, Trp, His, Pro)
Amino acids having aromatic side chains (Phe, Tyr, Trp)
Amino acids having acidic side chains (Asp, Glu)
Amino acids having basic side chains (Lys, Arg, His)
Amino acids having amide side chains (Asn, Gin)
Amino acids having hydroxy side chains (Ser, Thr)
Amino acids having sulphur-containing side chains (Cys, Met),
Neutral, weakly hydrophobic amino acids (Pro, Ala, Gly, Ser, Thr)
Hydrophilic, acidic amino acids (Gin, Asn, Glu, Asp), and
Hydrophobic amino acids (Leu, Ile, Val)

Particular preferred conservative amino acids substitution groups are: Val-Leu-Ile, Phe-Tyr, Lys-Arg, Ala-Val, and Asn-Gln.

In some embodiments, the antibody of the conjugates described herein comprises a heavy chain having an amino acid sequence with 80% or more amino acid sequence identity (for example, about 85% or more, 86% or more, 87% or more, 88% or more, 89% or more, 90% or more, 91% or more, 92% or more, 93% or more, 94% or more, 95% or more, 96% or more, 97% or more, 98% or more, 99% or more sequence identity) to a heavy chain described herein. In some embodiments, the antibody of the conjugates described herein comprises a light chain having an amino acid sequence with 80% or more amino acid sequence identity (for example, about 85% or more, 86% or more, 87% or more, 88% or more, 89% or more, 90% or more, 91% or more, 92% or more, 93% or more, 94% or more, 95% or more, 96% or more, 97% or more, 98% or more, 99% or more sequence identity) to a light chain described herein.

In some embodiments, the antibody of the conjugates described herein comprises a heavy chain having an amino acid sequence identical to the amino acid sequence of a heavy chain described herein, except that it includes 1, 2, 3, 4, 5, 6, 7, 8, 9 or 10 amino acid modifications (e.g., substitutions, insertions and/or deletions) relative to the amino acid sequence of the heavy chain described herein. In some embodiments, the antibody of the conjugates described herein comprises a light chain having an amino acid sequence identical to the amino acid sequence of a light chain described herein, except that it includes 1, 2, 3, 4, 5, 6, 7, 8, 9 or 10 amino acid modifications (e.g., substitutions, insertions and/or deletions) relative to the amino acid sequence of the light chain described herein.

Antibody Production

Humanized antibodies, fragments and regions can be produced by cloning DNA segments encoding the H and L chain antigen-binding regions of the anti-AXL antibody, and joining these DNA segments to DNA segments including CH and CL regions, respectively, to produce full length immunoglobulin-encoding genes.

For full-length antibody molecules, the immunoglobulin cDNAs can be obtained from mRNA of hybridoma cell lines. Antibody heavy and light chains are cloned in a mammalian expression vector system. Assembly is documented with DNA sequence analysis. The antibody construct can be expressed in human or other mammalian host cell lines. The construct can be validated by transient transfection assays and immunoassay of the expressed antibody. Stable cell lines with the highest productivity can be isolated and screened using rapid assay methods.

Functional Moieties

The humanised antibody of the disclosure may be conjugated to a functional moiety. Examples of functional moieties include an amino acid, a peptide, a protein, a polysaccharide, a nucleoside, a nucleotide, an oligonucleotide, a nucleic acid, a drug, a hormone, a lipid, a lipid assembly, a synthetic polymer, a polymeric microparticle, a biological cell, a virus, a reporter (such as a fluorophore, a chromophore, or a dye), a toxin, a hapten, an enzyme, a binding member (such as an antibody, or an antibody fragment), a radioisotope, solid matrixes, semisolid matrixes and combinations thereof, or an organic moiety.

Examples of a drug include a cytotoxic agent, a chemotherapeutic agent, a peptide, a peptidomimetic, a protein scaffold, DNA, RNA, siRNA, microRNA, and a peptidonucleic acid. In preferred embodiments the functional moiety is a PBD drug moiety. In other embodiments the humanised antibody is conjugated to a therapeutic agent or drug moiety that modifies a given biological response. Therapeutic agents or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (see, International Publication No. WO 97/33899), AIM II (see, International Publication No. WO 97/34911), Fas Ligand (Takahashi et al., 1994, J Immunol., 6: 1567), and VEGf (see, International Publication No. WO 99/23105), a thrombotic agent or an anti-angiogenic agent, e.g., angiostatin or endostatin; or, a biological response modifier such as, for example, a lymphokine (e.g., interleukin-1 (“IL-I”), interleukin-2 (“IL-2”), interleukin-4 (“IL-4”), interleukin-6 (“IL-6”), interleukin-7 (“IL-7”), interleukin-9 (“IL-9”), interleukin-15 (“IL-15”), interleukin-12 (“IL-12”), granulocyte macrophage colony stimulating factor (“GMCSF”), and granulocyte colony stimulating factor (“G-CSF”)), or a growth factor (e.g., growth hormone (“GH”)).

Examples of a reporter include a fluorophore, a chromophore, a radionuclide, and an enzyme. Such antibody-reporter conjugates can be useful for monitoring or prognosing the development or progression of a disorder (such as, but not limited to cancer) as part of a clinical testing procedure, such as determining the efficacy of a particular therapy. Such diagnosis and detection can accomplished by fusing or conjugating the antibody to detectable substances including, but not limited to various enzymes, such as but not limited to horseradish peroxidase, alkaline phosphatase, beta-galactosidase, or acetylcholinesterase; prosthetic groups, such as but not limited to streptavidin/biotin and avidin/biotin; fluorescent materials, such as but not limited to, umbelliferone, fluorescein, fluorescein isothiocynate, rhodamine, dichlorotriazinylamine fluorescein, dansyl chloride or phycoerythrin; luminescent materials, such as but not limited to, bioluminescent materials, such as but not limited to, luciferase, luciferin, and aequorin; radioactive materials, such as but not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho), indium (¹¹⁵In, ¹¹³In, ¹¹²In, ¹¹¹In), iodine (¹³¹I, ¹²⁵I, ¹²³I, ¹²¹I), lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (3⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and nonradioactive paramagnetic metal ions.

Examples of a binding member include an antibody or antibody fragment, and biotin and/or streptavidin.

A toxin, cytotoxin or cytotoxic agent includes any agent that is detrimental to cells. Examples of toxins include radioisotopes such as ¹³¹I, a ribosome inactivating protein such as pseudomonas exotoxin (PE38 fragment), plant or bacterial toxins such as ricin, the α-chain of ricin, saporin, pokeweed antiviral protein, diphtheria toxin, or Pseudomonas exotoxin A (Kreitman and Pastan (1998) Adv. Drug Delivery Rev. 31:53.). Other toxins and cytotoxins include, e.g., a cytostatic or cytocidal agent, or a radioactive metal ion, e.g., alpha-emitters. Examples include paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs or homo logs thereof, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). Chemical toxins can also be taken from the group chosen from duocarmycin (U.S. Pat. Nos. 5,703,080; 4,923,990), methotrexate, doxorubicin, melphalan, chlorambucil, ARA-C, vindesine, mitomycin C, cisplatinum, etoposide, bleomycin and 5-fluorouracil. Examples of chemotherapeutic agents also include Adriamycin, Doxorubicin, 5-Fluorouracil, Cytosine arabinoside (Ara-C), Cyclophosphamide, Thiotepa, Taxotere (docetaxel), Busulfan, Cytoxin, Taxol, Methotrexate, In one embodiment, the cytotoxic agent is chosen from an enediyne, a lexitropsin, a duocarmycin, a taxane, a puromycin, a dolastatin, a maytansinoid, and a vinca alkaloid. In other embodiments, the cytotoxic agent is paclitaxel, docetaxel, CC-I 065, SN-3 8, topotecan, morpholino-doxorubicin, rhizoxin, cyanomorpholino-doxorubicin, dolastatin-10, echinomycin, combretastatin, calicheamicin, maytansine, DM-I, an auristatin or other dolastatin derivatives, such as auristatin E or auristatin F, AEB, AEVB, AEFP, MMAE (monomethylauristatin E), MMAF (monomethylauristatin F), eleutherobin or netropsin. In certain embodiments, the cytoxic agent is Maytansine or Maytansinoids, and derivatives thereof, wherein an antibodies (full length or fragments) of the disclosure are conjugated to one or more maytansinoid molecules. Maytansinoids are mitototic inhibitors which act by inhibiting tubulin polymerization. In other embodimetns the toxin is a small molecule or protein toxins, such as, but not limited to abrin, brucine, cicutoxin, diphtheria toxin, batrachotoxin, botulism toxin, shiga toxin, endotoxin, Pseudomonas exotoxin, Pseudomonas endotoxin, tetanus toxin, pertussis toxin, anthrax toxin, cholera toxin, falcarinol, fumonisin BI, fumonisin B2, aflatoxin, maurotoxin, agitoxin, charybdotoxin, margatoxin, slotoxin, scyllatoxin, hefutoxin, calciseptine, taicatoxin, calcicludine, geldanamycin, gelonin, lotaustralin, ocratoxin A, patulin, ricin, strychnine, trichothecene, zearlenone, and tetradotoxin. Enzymatically active toxins and fragments thereof which can be used include diphtheria A chain, non-binding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, P APII, and PAP-S), Momordica charantia inhibitor, curcin, crotin, Sapaonaria officinalis inhibitor, gelonin, mitogellin, restrictocin, phenomycin, enomycin and the tricothecenes.

The humanized antibody may be modified by conjugation to an organic moiety. Such modification can produce an antibody or antigen-binding fragment with improved pharmacokinetic properties (e.g., increased in vivo serum half-life). The organic moiety can be a linear or branched hydrophilic polymeric group, fatty acid group, or fatty acid ester group. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms. In certain embodiments, the cytotoxic or cytostatic agent is a dolastatin. In more specific embodiments, the dolastatin is of the auristatin class. In a specific embodiment of the disclosure, the cytotoxic or cytostatic agent is MMAE. In another specific embodiment of the disclosure, the cytotoxic or cytostatic agent is AEFP. In another specific embodiment of the disclosure, the cytotoxic or cytostatic agent is MMAF.

The humanized antibody and antigen-binding fragments can comprise one or more organic moieties that are covalently bonded, directly or indirectly, to the antibody. Each organic moiety that is bonded to an antibody or antigen-binding fragment described herein can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, an antibody modified by the covalent attachment of polylysine is encompassed by the present disclosure. Hydrophilic polymers suitable for modifying antibodies described herein can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the antibody described herein has a molecular weight of about 800 to about 150,000 Daltons as a separate molecular entity. For example PEG5000 and PEG20,000, wherein the numerical component of the name is the average molecular weight of the polymer in Daltons, can be used. The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N,N-carbonyl diimidazole) on a fatty acid or fatty acid ester can be coupled to a hydroxyl group on a polymer.

Fatty acids and fatty acid esters suitable for modifying antibodies described herein can be saturated or can contain one or more units of unsaturation. Fatty acids that are suitable for modifying antibodies described herein include, for example, n-dodecanoate (C12, laurate), n-tetradecanoate (C14, myristate), n-octadecanoate (C18, stearate), n-eicosanoate (C20, arachidate), n-docosanoate (C22, behenate), n-triacontanoate (C30), n-tetracontanoate (C40), cis-δ 9-octadecanoate (C18, oleate), all cis-δ 5,8,11,14-eicosatetraenoate (C20, arachidonate), octanedioic acid, tetradecanedioic acid, octadecanedioic acid, docosanedioic acid, and similar fatty acids. Suitable fatty acid esters include mono-esters of dicarboxylic acids that comprise a linear or branched lower alkyl group. The lower alkyl group can comprise from one to about twelve, preferably one to about six, carbon atoms.

The above conjugates can be prepared using suitable methods, such as by reaction with one or more modifying agents: a “modifying agent” as the term is used herein, refers to a suitable organic group (e.g., hydrophilic polymer, a fatty acid, a fatty acid ester) that comprises an activating group; aAn “activating group” is a chemical moiety or functional group that can, under appropriate conditions, react with a second chemical group thereby forming a covalent bond between the modifying agent and the second chemical group.

For example, amine-reactive activating groups include electrophilic groups such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hernanson, G. T., Bioconjugate Techniques, Academic Press: San Diego, Calif. (1996)). An activating group can be bonded directly to the organic group (e.g., hydrophilic polymer, fatty acid, fatty acid ester), or through a linker moiety, for example a divalent C1-C12 group wherein one or more carbon atoms can be replaced by a heteroatom such as oxygen, nitrogen or sulfur. Suitable linker moieties include, for example, tetraethylene glycol, —(CH2)3-, —NH—(CH2)6-NH—, —(CH2)2-NH— and —CH2-O—CH2-CH2-O—CH2-CH2-O—CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate as described, or can be reacted with maleic anhydride and the resulting product cyclized to produce an activated maleimido derivative of the fatty acid. (See, for example, Thompson, et al., WO 92/16221 the entire teachings of which are incorporated herein by reference.)

The above conjugates can be produced by reacting a human antibody or antigen-binding fragment with a modifying agent. For example, the organic moieties can be bonded to the antibody in a non-site-specific manner by employing an amine-reactive modifying agent, for example, an NHS ester of PEG. Modified human antibodies or antigen-binding fragments can also be prepared by reducing disulfide bonds (e.g., inter-chain disulfide bonds) of an antibody or antigen-binding fragment. The reduced antibody or antigen-binding fragment can then be reacted with a thiol-reactive modifying agent to produce the modified antibody described herein. Modified human antibodies and antigen-binding fragments comprising an organic moiety that is bonded to specific sites of an antibody described herein can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6(10):2233-2241 (1997); Itoh et al., Bioorg. Chem., 24(1): 59-68 (1996); Capellas et al., Biotechnol. Bioeng., 56(4):456-463 (1997)), and the methods described in Hermanson, G. T., Bioconjugate Techniques, Academic Press: San Diego, Calif. (1996).

Pharmaceutically Acceptable Cations

Examples of pharmaceutically acceptable monovalent and divalent cations are discussed in Berge, et al., J. Pharm. Sci., 66, 1-19 (1977), which is incorporated herein by reference.

The pharmaceutically acceptable cation may be inorganic or organic.

Examples of pharmaceutically acceptable monovalent inorganic cations include, but are not limited to, alkali metal ions such as Na⁺ and K⁺. Examples of pharmaceutically acceptable divalent inorganic cations include, but are not limited to, alkaline earth cations such as Ca²⁺ and Mg²⁺. Examples of pharmaceutically acceptable organic cations include, but are not limited to, ammonium ion (i.e. NH₄⁺) and substituted ammonium ions (e.g. NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Substituents

The phrase “optionally substituted” as used herein, pertains to a parent group which may be unsubstituted or which may be substituted.

Unless otherwise specified, the term “substituted” as used herein, pertains to a parent group which bears one or more substituents. The term “substituent” is used herein in the conventional sense and refers to a chemical moiety which is covalently attached to, or if appropriate, fused to, a parent group. A wide variety of substituents are well known, and methods for their formation and introduction into a variety of parent groups are also well known.

Examples of substituents are described in more detail below.

C₁₋₁₂ alkyl: The term “C₁₋₁₂ alkyl” as used herein, pertains to a monovalent moiety obtained by removing a hydrogen atom from a carbon atom of a hydrocarbon compound having from 1 to 12 carbon atoms, which may be aliphatic or alicyclic, and which may be saturated or unsaturated (e.g. partially unsaturated, fully unsaturated). The term “C₁₋₄ alkyl” as used herein, pertains to a monovalent moiety obtained by removing a hydrogen atom from a carbon atom of a hydrocarbon compound having from 1 to 4 carbon atoms, which may be aliphatic or alicyclic, and which may be saturated or unsaturated (e.g. partially unsaturated, fully unsaturated). Thus, the term “alkyl” includes the sub-classes alkenyl, alkynyl, cycloalkyl, etc., discussed below.

Examples of saturated alkyl groups include, but are not limited to, methyl (C₁), ethyl (C₂), propyl (C₃), butyl (C₄), pentyl (C₅), hexyl (C₆) and heptyl (C₇).

Examples of saturated linear alkyl groups include, but are not limited to, methyl (C₁), ethyl (C₂), n-propyl (C₃), n-butyl (C₄), n-pentyl (amyl) (C₅), n-hexyl (C₆) and n-heptyl (C₇).

Examples of saturated branched alkyl groups include iso-propyl (C₃), iso-butyl (C₄), sec-butyl (C₄), tert-butyl (C₄), iso-pentyl (C₅), and neo-pentyl (C₅).

C₂₋₁₂ Alkenyl: The term “C₂₋₁₂ alkenyl” as used herein, pertains to an alkyl group having one or more carbon-carbon double bonds.

Examples of unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

C₂₋₁₂ alkynyl: The term “C₂₋₁₂ alkynyl” as used herein, pertains to an alkyl group having one or more carbon-carbon triple bonds.

Examples of unsaturated alkynyl groups include, but are not limited to, ethynyl (—C≡CH) and 2-propynyl (propargyl, —CH₂—C≡CH).

C₃₋₁₂ cycloalkyl: The term “C₃₋₁₂ cycloalkyl” as used herein, pertains to an alkyl group which is also a cyclyl group; that is, a monovalent moiety obtained by removing a hydrogen atom from an alicyclic ring atom of a cyclic hydrocarbon (carbocyclic) compound, which moiety has from 3 to 7 carbon atoms, including from 3 to 7 ring atoms.

Examples of cycloalkyl groups include, but are not limited to, those derived from:

Saturated Monocyclic Hydrocarbon Compounds:

cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇) and methylcyclohexane (C₇);

Unsaturated Monocyclic Hydrocarbon Compounds:

cyclopropene (C₃), cyclobutene (C₄), cyclopentene (C₀), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇) and methylcyclohexene (C₇); and

Saturated Polycyclic Hydrocarbon Compounds:

norcarane (C₇), norpinane (C₇), norbornane (C₇).

C₃₋₂₀ heterocyclyl: The term “C₃₋₂₀ heterocyclyl” as used herein, pertains to a monovalent moiety obtained by removing a hydrogen atom from a ring atom of a heterocyclic compound, which moiety has from 3 to 20 ring atoms, of which from 1 to 10 are ring heteroatoms. Preferably, each ring has from 3 to 7 ring atoms, of which from 1 to 4 are ring heteroatoms.

In this context, the prefixes (e.g. C₃₋₂₀, C₃₋₇, C₅₋₆, etc.) denote the number of ring atoms, or range of number of ring atoms, whether carbon atoms or heteroatoms. For example, the term “C₅₋₆heterocyclyl”, as used herein, pertains to a heterocyclyl group having 5 or 6 ring atoms.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₅) and oxathiane (thioxane) (C₆); and,

N₁O₁S₁: oxathiazine (C₆).

Examples of substituted monocyclic heterocyclyl groups include those derived from saccharides, in cyclic form, for example, furanoses (C₅), such as arabinofuranose, lyxofuranose, ribofuranose, and xylofuranse, and pyranoses (C₆), such as allopyranose, altropyranose, glucopyranose, mannopyranose, gulopyranose, idopyranose, galactopyranose, and talopyranose.

C₅₋₂₀ aryl: The term “C₅₋₂₀ aryl”, as used herein, pertains to a monovalent moiety obtained by removing a hydrogen atom from an aromatic ring atom of an aromatic compound, which moiety has from 3 to 20 ring atoms. The term “C₅₋₇ aryl”, as used herein, pertains to a monovalent moiety obtained by removing a hydrogen atom from an aromatic ring atom of an aromatic compound, which moiety has from 5 to 7 ring atoms and the term “C₅₋₁₀ aryl”, as used herein, pertains to a monovalent moiety obtained by removing a hydrogen atom from an aromatic ring atom of an aromatic compound, which moiety has from 5 to 10 ring atoms. Preferably, each ring has from 5 to 7 ring atoms.

In this context, the prefixes (e.g. C₃₋₂₀, C₅₋₇, C₅₋₆, C₅₋₁₀, etc.) denote the number of ring atoms, or range of number of ring atoms, whether carbon atoms or heteroatoms. For example, the term “C₅₋₆ aryl” as used herein, pertains to an aryl group having 5 or 6 ring atoms.

The ring atoms may be all carbon atoms, as in “carboaryl groups”. Examples of carboaryl groups include, but are not limited to, those derived from benzene (i.e. phenyl) (C₆), naphthalene (C₁₀), azulene (C₁₀), anthracene (C₁₄), phenanthrene (C₁₄), naphthacene (C₁₈), and pyrene (C₁₆).

Examples of aryl groups which comprise fused rings, at least one of which is an aromatic ring, include, but are not limited to, groups derived from indane (e.g. 2,3-dihydro-1H-indene) (C₉), indene (C₉), isoindene (C₉), tetraline (1,2,3,4-tetrahydronaphthalene (C₁₀), acenaphthene (C₁₂), fluorene (C₁₃), phenalene (C₁₃), acephenanthrene (C₁₅), and aceanthrene (C₁₆).

Alternatively, the ring atoms may include one or more heteroatoms, as in “heteroaryl groups”. Examples of monocyclic heteroaryl groups include, but are not limited to, those derived from:

N₁: pyrrole (azole) (C₅), pyridine (azine) (C₆);

O₁: furan (oxole) (C₅);

Si: thiophene (thiole) (C₅);

N₁O₁: oxazole (C₅), isoxazole (C₅), isoxazine (C₆);

N₂O₁: oxadiazole (furazan) (C₅);

N₃O₁: oxatriazole (C₅);

N₁S₁: thiazole (C₅), isothiazole (C₅);

N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);

N₃: triazole (C₅), triazine (C₆); and,

N₄: tetrazole (C₅).

Examples of heteroaryl which comprise fused rings, include, but are not limited to:
C₉ (with 2 fused rings) derived from benzofuran (O₁), isobenzofuran (O₁), indole (N₁), isoindole (N₁), indolizine (N₁), indoline (N₁), isoindoline (N₁), purine (N₄) (e.g., adenine, guanine), benzimidazole (N₂), indazole (N₂), benzoxazole (N₁O₁), benzisoxazole (N₁O₁), benzodioxole (O₂), benzofurazan (N₂O₁), benzotriazole (N₃), benzothiofuran (S₁), benzothiazole (N₁S₁), benzothiadiazole (N₂S);
C₁₀ (with 2 fused rings) derived from chromene (O₁), isochromene (O₁), chroman (O₁), isochroman (O₁), benzodioxan (O₂), quinoline (N₁), isoquinoline (N₁), quinolizine (N₁), benzoxazine (N₁O₁), benzodiazine (N₂), pyridopyridine (N₂), quinoxaline (N₂), quinazoline (N₂), cinnoline (N₂), phthalazine (N₂), naphthyridine (N₂), pteridine (N₄);
C₁₁ (with 2 fused rings) derived from benzodiazepine (N₂);
C₁₃ (with 3 fused rings) derived from carbazole (N₁), dibenzofuran (O₁), dibenzothiophene (S₁), carboline (N₂), perimidine (N₂), pyridoindole (N₂); and,
C₁₄ (with 3 fused rings) derived from acridine (N₁), xanthene (O₁), thioxanthene (S₁), oxanthrene (O₂), phenoxathiin (O₁S₁), phenazine (N₂), phenoxazine (N₁O₁), phenothiazine (N₁S₁), thianthrene (S₂), phenanthridine (N₁), phenanthroline (N₂), phenazine (N₂).

The above groups, whether alone or part of another substituent, may themselves optionally be substituted with one or more groups selected from themselves and the additional substituents listed below.

Halo: —F, —Cl, —Br, and —I.

Hydroxy: —OH.

Ether: —OR, wherein R is an ether substituent, for example, a C₁₋₇ alkyl group (also referred to as a C₁₋₇ alkoxy group, discussed below), a C₃₋₂₀ heterocyclyl group (also referred to as a C₃₋₂₀ heterocyclyloxy group), or a C₅₋₂₀ aryl group (also referred to as a C₅₋₂₀ aryloxy group), preferably a C₁₋₇alkyl group.

Alkoxy: —OR, wherein R is an alkyl group, for example, a C₁₋₇ alkyl group. Examples of C₁₋₇ alkoxy groups include, but are not limited to, —OMe (methoxy), —OEt (ethoxy), —O(nPr) (n-propoxy), —O(iPr) (isopropoxy), —O(nBu) (n-butoxy), —O(sBu) (sec-butoxy), —O(iBu) (isobutoxy), and —O(tBu) (tert-butoxy).

Acetal: —CH(OR¹)(OR²), wherein R¹ and R² are independently acetal substituents, for example, a C₁₋₇ alkyl group, a C₃₋₂₀ heterocyclyl group, or a C₅₋₂₀ aryl group, preferably a C₁₋₇ alkyl group, or, in the case of a “cyclic” acetal group, R¹ and R², taken together with the two oxygen atoms to which they are attached, and the carbon atoms to which they are attached, form a heterocyclic ring having from 4 to 8 ring atoms. Examples of acetal groups include, but are not limited to, —CH(OMe)₂, —CH(OEt)₂, and —CH(OMe)(OEt).

Hemiacetal: —CH(OH)(OR¹), wherein R¹ is a hemiacetal substituent, for example, a C₁₋₇ alkyl group, a C₃₋₂₀ heterocyclyl group, or a C₅₋₂₀ aryl group, preferably a C₁₋₇ alkyl group. Examples of hemiacetal groups include, but are not limited to, —CH(OH)(OMe) and —CH(OH)(OEt).

Ketal: —CR(OR¹)(OR²), where R¹ and R² are as defined for acetals, and R is a ketal substituent other than hydrogen, for example, a C₁₋₇ alkyl group, a C₃₋₂₀ heterocyclyl group, or a C₅₋₂₀ aryl group, preferably a C₁₋₇ alkyl group. Examples ketal groups include, but are not limited to, —C(Me)(OMe)₂, —C(Me)(OEt)₂, —C(Me)(OMe)(OEt), —C(Et)(OMe)₂, —C(Et)(OEt)₂, and —C(Et)(OMe)(OEt).

Hemiketal: —CR(OH)(OR¹), where R¹ is as defined for hemiacetals, and R is a hemiketal substituent other than hydrogen, for example, a C₁₋₇ alkyl group, a C₃₋₂₀ heterocyclyl group, or a C₅₋₂₀ aryl group, preferably a C₁₋₇ alkyl group. Examples of hemiacetal groups include, but are not limited to, —C(Me)(OH)(OMe), —C(Et)(OH)(OMe), —C(Me)(OH)(OEt), and —C(Et)(OH)(OEt).

Oxo (keto, -one): ═O.

Thione (thioketone): ═S.

Imino (imine): ═NR, wherein R is an imino substituent, for example, hydrogen, C₁₋₇ alkyl group, a C₃₋₂₀ heterocyclyl group, or a C₅₋₂₀ aryl group, preferably hydrogen or a C₁₋₇ alkyl group. Examples of ester groups include, but are not limited to, ═NH, ═NMe, ═NEt, and ═NPh.

Formyl (carbaldehyde, carboxaldehyde): —C(═O)H.

Acyl (keto): —C(═O)R, wherein R is an acyl substituent, for example, a C₁₋₇ alkyl group (also referred to as C₁₋₇ alkylacyl or C₁₋₇ alkanoyl), a C₃₋₂₀ heterocyclyl group (also referred to as C₃₋₂₀ heterocyclylacyl), or a C₅₋₂₀ aryl group (also referred to as C₅₋₂₀ arylacyl), preferably a C₁₋₇ alkyl group. Examples of acyl groups include, but are not limited to, —C(═O)CH₃ (acetyl), —C(═O)CH₂CH₃ (propionyl), —C(═O)C(CH₃)₃(t-butyryl), and —C(═O)Ph (benzoyl, phenone).

Carboxy (carboxylic acid): —C(═O)OH.

Thiocarboxy (thiocarboxylic acid): —C(═S)SH.

Thiolocarboxy (thiolocarboxylic acid): —C(═O)SH.

Thionocarboxy (thionocarboxylic acid): —C(═S)OH.

Imidic acid: —C(═NH)OH.

Hydroxamic acid: —C(═NOH)OH.

Ester (carboxylate, carboxylic acid ester, oxycarbonyl): —C(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —C(═O)OCH₃, —C(═O)OCH₂CH₃, —C(═O)OC(CH₃)₃, and —C(═O)OPh.

Acyloxy (reverse ester): —OC(═O)R, wherein R is an acyloxy substituent, for example, a C₁₋₇ alkyl group, a C₃₋₂₀ heterocyclyl group, or a C₅₋₂₀ aryl group, preferably a C₁₋₇ alkyl group. Examples of acyloxy groups include, but are not limited to, —OC(═O)CH₃ (acetoxy), —OC(═O)CH₂CH₃, —OC(═O)C(CH₃)₃, —OC(═O)Ph, and —OC(═O)CH₂Ph.

Oxycarboyloxy: —OC(═O)OR, wherein R is an ester substituent, for example, a C₁₋₇ alkyl group, a C₃₋₂₀ heterocyclyl group, or a C₅₋₂₀ aryl group, preferably a C₁₋₇ alkyl group. Examples of ester groups include, but are not limited to, —OC(═O)OCH₃, —OC(═O)OCH₂CH₃, —OC(═O)OC(CH₃)₃, and —OC(═O)OPh.

Amino: —NR¹R², wherein R¹ and R² are independently amino substituents, for example, hydrogen, a C₁₋₇ alkyl group (also referred to as C₁₋₇ alkylamino or di-C₁₋₇ alkylamino), a C₃₋₂₀ heterocyclyl group, or a C₅₋₂₀ aryl group, preferably H or a C₁₋₇ alkyl group, or, in the case of a “cyclic” amino group, R¹ and R², taken together with the nitrogen atom to which they are attached, form a heterocyclic ring having from 4 to 8 ring atoms. Amino groups may be primary (—NH₂), secondary (—NHR¹), or tertiary (—NHR¹R²), and in cationic form, may be quaternary (—NR¹R²R³). Examples of amino groups include, but are not limited to, —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

Amido (carbamoyl, carbamyl, aminocarbonyl, carboxamide): —C(═O)NR¹R², wherein R¹ and R² are independently amino substituents, as defined for amino groups. Examples of amido groups include, but are not limited

to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

Thioamido (thiocarbamyl): —C(═S)NR¹R², wherein R¹ and R² are independently amino substituents, as defined for amino groups. Examples of amido groups include, but are not limited to, —C(═S)NH₂, —C(═S)NHCH₃, —C(═S)N(CH₃)₂, and —C(═S)NHCH₂CH₃.

Acylamido (acylamino): —NR¹C(═O)R², wherein R¹ is an amide substituent, for example, hydrogen, a C1-7 alkyl group, a C₃₋₂₀ heterocyclyl group, or a C₅₋₂₀ aryl group, preferably hydrogen or a C₁₋₇ alkyl group, and R² is an acyl substituent, for example, a C₁₋₇ alkyl group, a C₃₋₂₀ heterocyclyl group, or a C₅₋₂₀aryl group, preferably hydrogen or a C₁₋₇ alkyl group. Examples of acylamide groups include, but are not limited to, —NHC(═O)CH₃, —NHC(═O)CH₂CH₃, and —NHC(═O)Ph. R¹ and R² may together form a cyclic structure, as in, for example, succinimidyl, maleimidyl, and phthalimidyl:

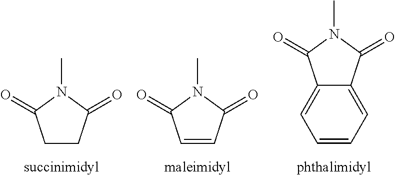

Aminocarbonyloxy: —OC(═O)NR¹R², wherein R¹ and R² are independently amino substituents, as defined for amino groups. Examples of aminocarbonyloxy groups include, but are not limited to, —OC(═O)NH₂, —OC(═O)NHMe, —OC(═O)NMe₂, and —OC(═O)NEt₂.

Ureido: —N(R¹)CONR²R³ wherein R² and R³ are independently amino substituents, as defined for amino groups, and R¹ is a ureido substituent, for example, hydrogen, a C₁₋₇ alkyl group, a C₃₋₂₀ heterocyclyl group, or a C₅₋₂₀ aryl group, preferably hydrogen or a C₁₋₇ alkyl group. Examples of ureido groups include, but are not limited to, —NHCONH₂, —NHCONHMe, —NHCONHEt, —NHCONMe₂, —NHCONEt₂, —NMeCONH₂, —NMeCONHMe, —NMeCONHEt, —NMeCONMe₂, and —NMeCONEt₂.

Guanidino: —NH—C(═NH)NH₂.

Tetrazolyl: a five membered aromatic ring having four nitrogen atoms and one carbon atom,

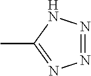

Imino: ═NR, wherein R is an imino substituent, for example, for example, hydrogen, a C₁₋₇ alkyl group, a C₃₋₂₀ heterocyclyl group, or a C₅₋₂₀ aryl group, preferably H or a C₁₋₇alkyl group. Examples of imino groups include, but are not limited to, ═NH, ═NMe, and ═NEt.

Amidine (amidino): —C(═NR)NR₂, wherein each R is an amidine substituent, for example, hydrogen, a C₁₋₇ alkyl group, a C₃₋₂₀ heterocyclyl group, or a C₅₋₂₀ aryl group, preferably H or a C₁₋₇ alkyl group. Examples of amidine groups include, but are not limited to, —C(═NH)NH₂, —C(═NH)NMe₂, and —C(═NMe)NMe₂.

Nitro: —NO₂.

Nitroso: —NO.

Azido: —N₃.

Cyano (nitrile, carbonitrile): —CN.

Isocyano: —NC.

Cyanato: —OCN.

Isocyanato: —NCO.

Thiocyano (thiocyanato): —SCN.

Isothiocyano (isothiocyanato): —NCS.

Sulfhydryl (thiol, mercapto): —SH.

Thioether (sulfide): —SR, wherein R is a thioether substituent, for example, a C₁₋₇ alkyl group (also referred to as a C₁₋₇alkylthio group), a C₃₋₂₀ heterocyclyl group, or a C₅₋₂₀ aryl group, preferably a C₁₋₇ alkyl group. Examples of C₁₋₇ alkylthio groups include, but are not limited to, —SCH₃ and —SCH₂CH₃.

Disulfide: —SS—R, wherein R is a disulfide substituent, for example, a C₁₋₇ alkyl group, a C₃₋₂₀ heterocyclyl group, or a C₅₋₂₀ aryl group, preferably a C₁₋₇ alkyl group (also referred to herein as C₁₋₇ alkyl disulfide). Examples of C₁₋₇ alkyl disulfide groups include, but are not limited to, —SSCH₃ and —SSCH₂CH₃.

Sulfine (sulfinyl, sulfoxide): —S(═O)R, wherein R is a sulfine substituent, for example, a C₁₋₇ alkyl group, a C₃₋₂₀ heterocyclyl group, or a C₅₋₂₀ aryl group, preferably a C₁₋₇ alkyl group. Examples of sulfine groups include, but are not limited to, —S(═O)CH₃ and —S(═O)CH₂CH₃.

Sulfone (sulfonyl): —S(═O)₂R, wherein R is a sulfone substituent, for example, a C₁₋₇ alkyl group, a C₃₋₂₀ heterocyclyl group, or a C₅₋₂₀ aryl group, preferably a C₁₋₇ alkyl group, including, for example, a fluorinated or perfluorinated C₁₋₇ alkyl group. Examples of sulfone groups include, but are not limited to, —S(═O)₂CH₃ (methanesulfonyl, mesyl), —S(═O)₂CF₃ (triflyl), —S(═O)₂CH₂CH₃ (esyl), —S(═O)₂C₄F₉ (nonaflyl), —S(═O)₂CH₂CF₃ (tresyl), —S(═O)₂CH₂CH₂NH₂ (tauryl), —S(═O)₂Ph (phenylsulfonyl, besyl), 4-methylphenylsulfonyl (tosyl), 4-chlorophenylsulfonyl (closyl), 4-bromophenylsulfonyl (brosyl), 4-nitrophenyl (nosyl), 2-naphthalenesulfonate (napsyl), and 5-dimethylamino-naphthalen-1-ylsulfonate (dansyl).

Sulfinic acid (sulfino): —S(═O)OH, —SO₂H.

Sulfonic acid (sulfo): —S(═O)₂OH, —SO₃H.

Sulfinate (sulfinic acid ester): —S(═O)OR; wherein R is a sulfinate substituent, for example, a C₁₋₇ alkyl group, a C₃₋₂₀ heterocyclyl group, or a C₅₋₂₀ aryl group, preferably a C₁₋₇ alkyl group. Examples of sulfinate groups include, but are not limited to, —S(═O)OCH₃ (methoxysulfinyl; methyl sulfinate) and —S(═O)OCH₂CH₃ (ethoxysulfinyl; ethyl sulfinate).

Sulfonate (sulfonic acid ester): —S(═O)₂OR, wherein R is a sulfonate substituent, for example, a C₁₋₇ alkyl group, a C₃₋₂₀ heterocyclyl group, or a C₅₋₂₀ aryl group, preferably a C₁₋₇ alkyl group. Examples of sulfonate groups include, but are not limited to, —S(═O)₂OCH₃ (methoxysulfonyl; methyl sulfonate) and —S(═O)₂OCH₂CH₃ (ethoxysulfonyl; ethyl sulfonate).

Sulfinyloxy: —OS(═O)R, wherein R is a sulfinyloxy substituent, for example, a C₁₋₇ alkyl group, a C₃₋₂₀ heterocyclyl group, or a C₅₋₂₀ aryl group, preferably a C₁₋₇ alkyl group. Examples of sulfinyloxy groups include, but are not limited to, —OS(═O)CH₃ and —OS(═O)CH₂CH₃.

Sulfonyloxy: —OS(═O)₂R, wherein R is a sulfonyloxy substituent, for example, a C₁₋₇ alkyl group, a C₃₋₂₀ heterocyclyl group, or a C₅₋₂₀ aryl group, preferably a C₁₋₇ alkyl group. Examples of sulfonyloxy groups include, but are not limited to, —OS(═O)₂CH₃ (mesylate) and —OS(═O)₂CH₂CH₃ (esylate).

Sulfate: —OS(═O)₂OR; wherein R is a sulfate substituent, for example, a C₁₋₇ alkyl group, a C₃₋₂₀ heterocyclyl group, or a C5-20 aryl group, preferably a C₁₋₇ alkyl group. Examples of sulfate groups include, but are not limited to, —OS(═O)₂OCH₃ and —SO(═O)₂OCH₂CH₃.

Sulfamyl (sulfamoyl; sulfinic acid amide; sulfinamide): —S(═O)NR¹R², wherein R¹ and R² are independently amino substituents, as defined for amino groups. Examples of sulfamyl groups include, but are not limited

to, —S(═O)NH₂, —S(═O)NH(CH₃), —S(═O)N(CH₃)₂, —S(═O)NH(CH₂CH₃), —S(═O)N(CH₂CH₃)₂, and —S(═O)NHPh.

Sulfonamido (sulfinamoyl; sulfonic acid amide; sulfonamide): —S(═O)₂NR¹R², wherein R¹ and R² are independently amino substituents, as defined for amino groups. Examples of sulfonamido groups include, but are not limited

to, —S(═O)₂NH₂, —S(═O)₂NH(CH₃), —S(═O)₂N(CH₃)₂, —S(═O)₂NH(CH₂CH₃), —S(═O)₂N(CH₂CH₃)₂, and —S(═O)₂NHPh.

Sulfamino: —NR¹S(═O)₂OH, wherein R¹ is an amino substituent, as defined for amino groups. Examples of sulfamino groups include, but are not limited to, —NHS(═O)₂OH and —N(CH₃)S(═O)₂OH.

Sulfonamino: —NR¹S(═O)₂R, wherein R¹ is an amino substituent, as defined for amino groups, and R is a sulfonamino substituent, for example, a C₁₋₇ alkyl group, a C₃₋₂₀ heterocyclyl group, or a C₅₋₂₀ aryl group, preferably a C₁₋₇ alkyl group. Examples of sulfonamino groups include, but are not limited to, —NHS(═O)₂CH₃ and —N(CH₃)S(═O)₂C₆H₅.

Sulfinamino: —NR¹S(═O)R, wherein R¹ is an amino substituent, as defined for amino groups, and R is a sulfinamino substituent, for example, a C₁₋₇ alkyl group, a C₃₋₂₀ heterocyclyl group, or a C₅₋₂₀ aryl group, preferably a C₁₋₇ alkyl group. Examples of sulfinamino groups include, but are not limited to, —NHS(═O)CH₃ and —N(CH₃)S(═O)C₆H₅.

Phosphino (phosphine): —PR₂, wherein R is a phosphino substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphino groups include, but are not limited to, —PH₂, —P(CH₃)₂, —P(CH₂CH₃)₂, —P(t-Bu)₂, and —P(Ph)₂.

Phospho: —P(═O)₂.

Phosphinyl (phosphine oxide): —P(═O)R₂, wherein R is a phosphinyl substituent, for example, a C₁₋₇ alkyl group, a C₃₋₂₀ heterocyclyl group, or a C₅₋₂₀ aryl group, preferably a C₁₋₇ alkyl group or a C₅₋₂₀ aryl group. Examples of phosphinyl groups include, but are not limited to, —P(═O)(CH₃)₂, —P(═O)(CH₂CH₃)₂, —P(═O)(t-Bu)₂, and —P(═O)(Ph)₂.

Phosphonic acid (phosphono): —P(═O)(OH)₂.

Phosphonate (phosphono ester): —P(═O)(OR)₂, where R is a phosphonate substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphonate groups include, but are not limited to, —P(═O)(OCH₃)₂, —P(═O)(OCH₂CH₃)₂, —P(═O)(O-t-Bu)₂, and —P(═O)(OPh)₂.

Phosphoric acid (phosphonooxy): —OP(═O)(OH)₂.

Phosphate (phosphonooxy ester): —OP(═O)(OR)₂, where R is a phosphate substituent, for example, —H, a C₁₋₇ alkyl group, a C₃₋₂₀ heterocyclyl group, or a C5-20 aryl group, preferably —H, a C₁₋₇ alkyl group, or a C5-20 aryl group. Examples of phosphate groups include, but are not limited to, —OP(═O)(OCH₃)₂, —OP(═O)(OCH₂CH₃)₂, —OP(═O)(O-t-Bu)₂, and —OP(═O)(OPh)₂.

Phosphorous acid: —OP(OH)₂.

Phosphite: —OP(OR)₂, where R is a phosphite substituent, for example, —H, a C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphite groups include, but are not limited

to, —OP(OCH₃)₂, —OP(OCH₂CH₃)₂, —OP(O-t-Bu)₂, and —OP(OPh)₂.

Phosphoramidite: —OP(OR¹)—NR²₂, where R¹ and R² are phosphoramidite substituents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidite groups include, but are not limited to, —OP(OCH₂CH₃)—N(CH₃)₂, —OP(OCH₂CH₃)—N(i-Pr)₂, and —OP(OCH₂CH₂CN)—N(i-Pr)₂.

Phosphoramidate: —OP(═O)(OR¹)—NR²₂, where R¹ and R² are phosphoramidate substituents, for example, —H, a (optionally substituted) C₁₋₇ alkyl group, a C₃₋₂₀ heterocyclyl group, or a C₅₋₂₀ aryl group, preferably —H, a C₁₋₇ alkyl group, or a C₅₋₂₀ aryl group. Examples of phosphoramidate groups include, but are not limited to, —OP(═O)(OCH₂CH₃)—N(CH₃)₂, —OP(═O)(OCH₂CH₃)—N(i-Pr)₂, and —OP(═O)(OCH₂CH₂CN)—N(i-Pr)₂.

Alkylene

C₃₋₁₂ alkylene: The term “C₃₋₁₂ alkylene”, as used herein, pertains to a bidentate moiety obtained by removing two hydrogen atoms, either both from the same carbon atom, or one from each of two different carbon atoms, of a hydrocarbon compound having from 3 to 12 carbon atoms (unless otherwise specified), which may be aliphatic or alicyclic, and which may be saturated, partially unsaturated, or fully unsaturated. Thus, the term “alkylene” includes the sub-classes alkenylene, alkynylene, cycloalkylene, etc., discussed below.

Examples of linear saturated C₃₋₁₂ alkylene groups include, but are not limited to, —(CH₂)ₙ— where n is an integer from 3 to 12, for example, —CH₂CH₂CH₂-(propylene), —CH₂CH₂CH₂CH₂— (butylene), —CH₂CH₂CH₂CH₂CH₂— (pentylene) and —CH₂CH₂CH₂CH-₂CH₂CH₂CH₂— (heptylene).

Examples of branched saturated C₃₋₁₂ alkylene groups include, but are not limited to, —CH(CH₃)CH₂—, —CH(CH₃)CH₂CH₂—, —CH(CH₃)CH₂CH₂CH₂—, —CH₂CH(CH₃)CH₂—, —CH₂CH(CH₃)CH₂CH₂—, —CH(CH₂CH₃)—, —CH(CH₂CH₃)CH₂—, and —CH₂CH(CH₂CH₃)CH₂—.

Examples of linear partially unsaturated C₃₋₁₂ alkylene groups (C₃₋₁₂ alkenylene, and alkynylene groups) include, but are not limited to, —CH═CH—CH₂—, —CH₂—CH═CH₂—, —CH═CH—CH₂—CH₂—, —CH═CH—CH₂—CH₂—CH₂—, —CH═CH—CH═CH—, —CH═CH—CH═CH—CH₂—, —CH═CH—CH═CH—CH₂—CH₂—, —CH═CH—CH₂—CH═CH—, —CH═CH—CH₂—CH₂—CH═CH—, and —CH₂—C≡C—CH₂—.

Examples of branched partially unsaturated C₃₋₁₂ alkylene groups (C₃₋₁₂ alkenylene and alkynylene groups) include, but are not limited to, —C(CH₃)═CH—, —C(CH₃)═CH—CH₂—, —CH═CH—CH(CH₃)— and —C═C—CH(CH₃)—.

Examples of alicyclic saturated C₃₋₁₂ alkylene groups (C₃₋₁₂ cycloalkylenes) include, but are not limited to, cyclopentylene (e.g. cyclopent-1,3-ylene), and cyclohexylene (e.g. cyclohex-1,4-ylene).

Examples of alicyclic partially unsaturated C₃₋₁₂ alkylene groups (C₃₋₁₂ cycloalkylenes) include, but are not limited to, cyclopentenylene (e.g. 4-cyclopenten-1,3-ylene), cyclohexenylene (e.g. 2-cyclohexen-1,4-ylene; 3-cyclohexen-1,2-ylene; 2,5-cyclohexadien-1,4-ylene).

Carbamate nitrogen protecting group: the term “carbamate nitrogen protecting group” pertains to a moiety which masks the nitrogen in the imine bond, and these are well known in the art. These groups have the following structure:

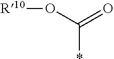

wherein R^{′10} is R as defined above. A large number of suitable groups are described on pages 503 to 549 of Greene, T. W. and Wuts, G. M., Protective Groups in Organic Synthesis, 3^{rd} Edition, John Wiley & Sons, Inc., 1999, which is incorporated herein by reference.

Hemi-aminal nitrogen protecting group: the term “hemi-aminal nitrogen protecting group” pertains to a group having the following structure:

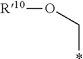

wherein R^{′10} is R as defined above. A large number of suitable groups are described on pages 633 to 647 as amide protecting groups of Greene, T. W. and Wuts, G. M., Protective Groups in Organic Synthesis, 3^{rd} Edition, John Wiley & Sons, Inc., 1999, which is incorporated herein by reference.

The groups Carbamate nitrogen protecting group and Hemi-aminal nitrogen protecting group may be jointly termed a “nitrogen protecting group for synthesis”.

Antibody-PBD Conjugates

The present disclosure provides a conjugate comprising a PBD compound connected to the antibody via a Linker Unit.

In one embodiment, the conjugate comprises the antibody connected to a spacer connecting group, the spacer connected to a trigger, the trigger connected to a self-immolative linker, and the self-immolative linker connected to the N10 position of the PBD compound. Such a conjugate is illustrated below:

where Ab is the antibody as defined above and PBD is a pyrrolobenzodiazepine compound (D), as described herein. The illustration shows the portions that correspond to R^{L′}, A, L¹ and L² in certain embodiments of the disclosure. R^{L′} may be either R^{L1′} or R^{L2′}. D is D^{L} with R^{L1′} or R^{L2′} removed.

The present disclosure is suitable for use in providing a PBD compound to a preferred site in a subject. In the preferred embodiments, the conjugate allows the release of an active PBD compound that does not retain any part of the linker. There is no stub present that could affect the reactivity of the PBD compound.

The linker attaches the antibody to the PBD drug moiety D through covalent bond(s). The linker is a bifunctional or multifunctional moiety which can be used to link one or more drug moiety (D) and an antibody unit (Ab) to form antibody-drug conjugates (ADC). The linker (R^{L′}) may be stable outside a cell, i.e. extracellular, or it may be cleavable by enzymatic activity, hydrolysis, or other metabolic conditions. Antibody-drug conjugates (ADC) can be conveniently prepared using a linker having reactive functionality for binding to the drug moiety and to the antibody. A cysteine thiol, or an amine, e.g. N-terminus or amino acid side chain such as lysine, of the antibody (Ab) can form a bond with a functional group of a linker or spacer reagent, PBD drug moiety (D) or drug-linker reagent (D^{L}, D—R^{L}), where R^{L} can be R^{L1} or R^{L2}.

The linkers of the ADC preferably prevent aggregation of ADC molecules and keep the ADC freely soluble in aqueous media and in a monomeric state.

The linkers of the ADC are preferably stable extracellularly. Before transport or delivery into a cell, the antibody-drug conjugate (ADC) is preferably stable and remains intact, i.e. the antibody remains linked to the drug moiety. The linkers are stable outside the target cell and may be cleaved at some efficacious rate inside the cell. An effective linker will: (i) maintain the specific binding properties of the antibody; (ii) allow intracellular delivery of the conjugate or drug moiety; (iii) remain stable and intact, i.e. not cleaved, until the conjugate has been delivered or transported to its targetted site; and (iv) maintain a cytotoxic, cell-killing effect or a cytostatic effect of the PBD drug moiety. Stability of the ADC may be measured by standard analytical techniques such as mass spectroscopy, HPLC, and the separation/analysis technique LC/MS.

Covalent attachment of the antibody and the drug moiety requires the linker to have two reactive functional groups, i.e. bivalency in a reactive sense. Bivalent linker reagents which are useful to attach two or more functional or biologically active moieties, such as peptides, nucleic acids, drugs, toxins, antibodies, haptens, and reporter groups are known, and methods have been described their resulting conjugates (Hermanson, G. T. (1996) Bioconjugate Techniques; Academic Press: New York, p 234-242).

In another embodiment, the linker may be substituted with groups which modulate aggregation, solubility or reactivity. For example, a sulfonate substituent may increase water solubility of the reagent and facilitate the coupling reaction of the linker reagent with the antibody or the drug moiety, or facilitate the coupling reaction of Ab-L with D^{L}, or DL-L with Ab, depending on the synthetic route employed to prepare the ADC.

In one embodiment, L-R^{L′} is a group:

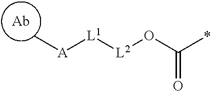

where the asterisk indicates the point of attachment to the Drug Unit (D), Ab is the antibody (L), L¹ is a linker, A is a connecting group connecting L¹ to the antibody, L² is a covalent bond or together with —OC(═O)— forms a self-immolative linker, and L¹ or L² is a cleavable linker.

L¹ is preferably the cleavable linker, and may be referred to as a trigger for activation of the linker for cleavage.

The nature of L¹ and L², where present, can vary widely. These groups are chosen on the basis of their cleavage characteristics, which may be dictated by the conditions at the site to which the conjugate is delivered. Those linkers that are cleaved by the action of enzymes are preferred, although linkers that are cleavable by changes in pH (e.g. acid or base labile), temperature or upon irradiation (e.g. photolabile) may also be used. Linkers that are cleavable under reducing or oxidising conditions may also find use in the present disclosure.

L¹ may comprise a contiguous sequence of amino acids. The amino acid sequence may be the target substrate for enzymatic cleavage, thereby allowing release of L-R^{L′} from the N10 position.

In one embodiment, L¹ is cleavable by the action of an enzyme. In one embodiment, the enzyme is an esterase or a peptidase.

In one embodiment, L² is present and together with —C(═O)O— forms a self-immolative linker.

In one embodiment, L² is a substrate for enzymatic activity, thereby allowing release of L-R^{L′} from the N10 position.

In one embodiment, where L¹ is cleavable by the action of an enzyme and L² is present, the enzyme cleaves the bond between L¹ and L².

L¹ and L², where present, may be connected by a bond selected from:
—C(═O)NH—,
—C(═O)O—,
—NHC(═O)—,
—OC(═O)—,
—OC(═O)O—,
—NHC(═O)O—,
—OC(═O)NH—, and
—NHC(═O)NH—.

An amino group of L¹ that connects to L² may be the N-terminus of an amino acid or may be derived from an amino group of an amino acid side chain, for example a lysine amino acid side chain.

A carboxyl group of L¹ that connects to L² may be the C-terminus of an amino acid or may be derived from a carboxyl group of an amino acid side chain, for example a glutamic acid amino acid side chain.

A hydroxyl group of L¹ that connects to L² may be derived from a hydroxyl group of an amino acid side chain, for example a serine amino acid side chain.

The term “amino acid side chain” includes those groups found in: (i) naturally occurring amino acids such as alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine; (ii) minor amino acids such as ornithine and citrulline; (iii) unnatural amino acids, beta-amino acids, synthetic analogs and derivatives of naturally occurring amino acids; and (iv) all enantiomers, diastereomers, isomerically enriched, isotopically labelled (e.g. ²H, ³H, ¹⁴C, ¹⁵N), protected forms, and racemic mixtures thereof.

In one embodiment, —C(═O)O— and L² together form the group:

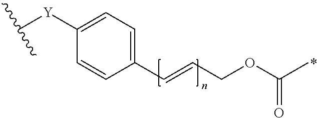

where the asterisk indicates the point of attachment to the N10 position, the wavy line indicates the point of attachment to the linker L¹, Y is —N(H)—, —O—, —C(═O)N(H)— or —C(═O)O—, and n is 0 to 3. The phenylene ring is optionally substituted with one, two or three substituents as described herein. In one embodiment, the phenylene group is optionally substituted with halo, NO₂, R or OR.

In one embodiment, Y is NH.

In one embodiment, n is 0 or 1. Preferably, n is 0.

Where Y is NH and n is 0, the self-immolative linker may be referred to as a p-aminobenzylcarbonyl linker (PABC).

The self-immolative linker will allow for release of the protected compound when a remote site is activated, proceeding along the lines shown below (for n=0):

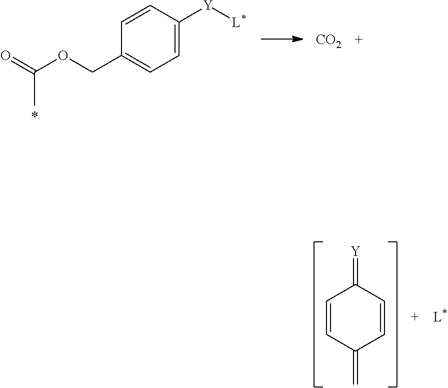

where L* is the activated form of the remaining portion of the linker. These groups have the advantage of separating the site of activation from the compound being protected.

As described above, the phenylene group may be optionally substituted.

In one embodiment described herein, the group L* is a linker L¹ as described herein, which may include a dipeptide group.

In another embodiment, —C(═O)O— and L² together form a group selected from:

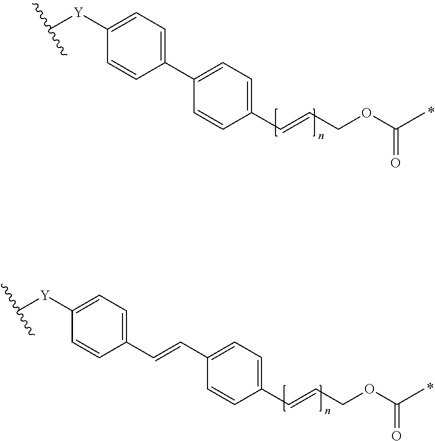

where the asterisk, the wavy line, Y, and n are as defined above. Each phenylene ring is optionally substituted with one, two or three substituents as described herein. In one embodiment, the phenylene ring having the Y substituent is optionally substituted and the phenylene ring not having the Y substituent is unsubstituted. In one embodiment, the phenylene ring having the Y substituent is unsubstituted and the phenylene ring not having the Y substituent is optionally substituted.

In another embodiment, —C(═O)O— and L² together form a group selected from:

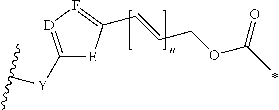

where the asterisk, the wavy line, Y, and n are as defined above, E is O, S or NR, D is N, CH, or CR, and F is N, CH, or CR.

In one embodiment, D is N.

In one embodiment, D is CH.

In one embodiment, E is O or S.

In one embodiment, F is CH.

In a preferred embodiment, the linker is a cathepsin labile linker.

In one embodiment, L¹ comprises a dipeptide The dipeptide may be represented as —NH—X₁—X₂—CO—, where —NH— and —CO— represent the N- and C-terminals of the amino acid groups X₁ and X₂ respectively. The amino acids in the dipeptide may be any combination of natural amino acids. Where the linker is a cathepsin labile linker, the dipeptide may be the site of action for cathepsin-mediated cleavage.

Additionally, for those amino acids groups having carboxyl or amino side chain functionality, for example Glu and Lys respectively, CO and NH may represent that side chain functionality.

In one embodiment, the group —X1-X₂— in dipeptide, —NH—X₁—X₂—CO—, is selected from:
Phe-Lys-,
Val-Ala-,
Val-Lys-,
Ala-Lys-,
Val-Cit-,
Phe-Cit-,
Leu-Cit-,
Ile-Cit-,
Phe-Arg-,
Trp-Cit-

where Cit is citrulline.

Preferably, the group —X₁—X₂— in dipeptide, —NH—X₁—X₂—CO—, is selected from:
Phe-Lys-,
Val-Ala-,
Val-Lys-,
Ala-Lys-,
Val-Cit-.

Most preferably, the group —X₁—X₂— in dipeptide, —NH—X₁—X₂—CO—, is -Phe-Lys- or -Val-Ala-.

Other dipeptide combinations may be used, including those described by Dubowchik et al., Bioconjugate Chemistry, 2002, 13, 855-869, which is incorporated herein by reference.

In one embodiment, the amino acid side chain is derivatised, where appropriate. For example, an amino group or carboxy group of an amino acid side chain may be derivatised. In one embodiment, an amino group NH₂ of a side chain amino acid, such as lysine, is a derivatised form selected from the group consisting of NHR and NRR′.

In one embodiment, a carboxy group COOH of a side chain amino acid, such as aspartic acid, is a derivatised form selected from the group consisting of COOR, CONH₂, CONHR and CONRR′.

In one embodiment, the amino acid side chain is chemically protected, where appropriate. The side chain protecting group may be a group as discussed below in relation to the group R^{L}. The present inventors have established that protected amino acid sequences are cleavable by enzymes. For example, it has been established that a dipeptide sequence comprising a Boc side chain-protected Lys residue is cleavable by cathepsin.

Protecting groups for the side chains of amino acids are well known in the art and are described in the Novabiochem Catalog. Additional protecting group strategies are set out in Protective Groups in Organic Synthesis, Greene and Wuts.

Possible side chain protecting groups are shown below for those amino acids having reactive side chain functionality:
Arg: Z, Mtr, Tos;
Asn: Trt, Xan;
Asp: Bzl, t-Bu;
Cys: Acm, Bzl, Bzl-OMe, Bzl-Me, Trt;
Glu: Bzl, t-Bu;
Gin: Trt, Xan;
His: Boc, Dnp, Tos, Trt;
Lys: Boc, Z—Cl, Fmoc, Z, Alloc;
Ser: Bzl, TBDMS, TBDPS;
Thr: Bz;
Trp: Boc;
Tyr: Bzl, Z, Z—Br.

In one embodiment, the side chain protection is selected to be orthogonal to a group provided as, or as part of, a capping group, where present. Thus, the removal of the side chain protecting group does not remove the capping group, or any protecting group functionality that is part of the capping group.

In other embodiments of the disclosure, the amino acids selected are those having no reactive side chain functionality. For example, the amino acids may be selected from: Ala, Gly, Ile, Leu, Met, Phe, Pro, and Val.

In one embodiment, the dipeptide is used in combination with a self-immolative linker. The self-immolative linker may be connected to —X₂—.

Where a self-immolative linker is present, —X₂— is connected directly to the self-immolative linker. Preferably the group —X₂—CO— is connected to Y, where Y is NH, thereby forming the group —X₂—CO—NH—.

—NH—X₁— is connected directly to A. A may comprise the functionality —CO— thereby to form an amide link with —X₁—.

In one embodiment, L¹ and L² together with —OC(═O)— comprise the group NH—X₁—X₂—CO-PABC-. The PABC group is connected directly to the N10 position.

Preferably, the self-immolative linker and the dipeptide together form the group —NH-Phe-Lys-CO—NH-PABC-, which is illustrated below:

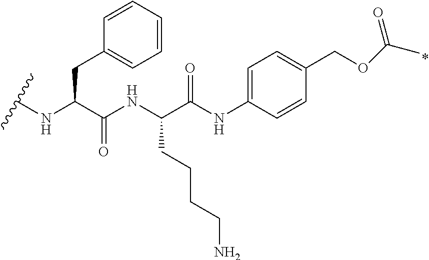 [SMILES: *N[C@@H](Cc1ccccc1)C(=O)N[C@@H](CCCCN)C(=O)Nc1ccc(COC(C)=O)cc1]

where the asterisk indicates the point of attachment to the N10 position, and the wavy line indicates the point of attachment to the remaining portion of the linker L¹ or the point of attachment to A. Preferably, the wavy line indicates the point of attachment to A. The side chain of the Lys amino acid may be protected, for example, with Boc, Fmoc, or Alloc, as described above.

Alternatively, the self-immolative linker and the dipeptide together form the group —NH-Val-Ala-CO—NH-PABC-, which is illustrated below:

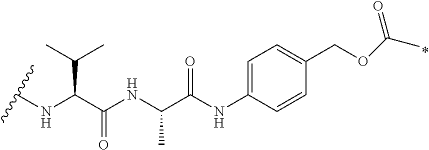 [SMILES: *N[C@H](C(=O)N[C@@H](C)C(=O)Nc1ccc(COC(C)=O)cc1)C(C)C]

where the asterisk and the wavy line are as defined above.

Alternatively, the self-immolative linker and the dipeptide together form the group —NH-Val-Cit-CO—NH-PABC-, which is illustrated below:

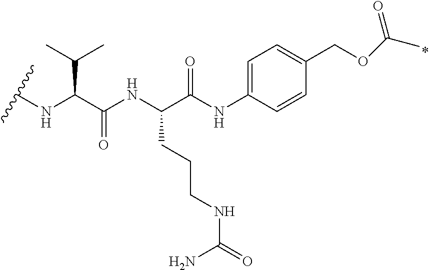 [SMILES: *N[C@H](C(=O)N[C@@H](CCCNC(N)=O)C(=O)Nc1ccc(COC(C)=O)cc1)C(C)C]

where the asterisk and the wavy line are as defined above.

In one embodiment, A is a covalent bond. Thus, L¹ and the antibody are directly connected.

For example, where L¹ comprises a contiguous amino acid sequence, the N-terminus of the sequence may connect directly to the antibody.

Thus, where A is a covalent bond, the connection between the antibody and L¹ may be selected from:
—C(═O)NH—,
—C(═O)O—,
—NHC(═O)—,
—OC(═O)—,
—OC(═O)O—,
—NHC(═O)O—,
—OC(═O)NH—,
—NHC(═O)NH—,
—C(═O)NHC(═O)—,
—S—,
—S—S—,
—CH₂C(═O)—, and
═N—NH—.

An amino group of L¹ that connects to the antibody may be the N-terminus of an amino acid or may be derived from an amino group of an amino acid side chain, for example a lysine amino acid side chain.

An carboxyl group of L¹ that connects to the antibody may be the C-terminus of an amino acid or may be derived from a carboxyl group of an amino acid side chain, for example a glutamic acid amino acid side chain.

A hydroxyl group of L¹ that connects to the antibody may be derived from a hydroxyl group of an amino acid side chain, for example a serine amino acid side chain.

A thiol group of L¹ that connects to the antibody may be derived from a thiol group of an amino acid side chain, for example a serine amino acid side chain.

The comments above in relation to the amino, carboxyl, hydroxyl and thiol groups of L¹ also apply to the antibody.

In one embodiment, L² together with —OC(═O)— represents:

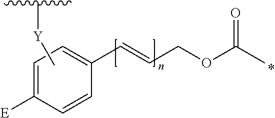

where the asterisk indicates the point of attachment to the N10 position, the wavy line indicates the point of attachment to L¹, n is 0 to 3, Y is a covalent bond or a functional group, and E is an activatable group, for example by enzymatic action or light, thereby to generate a self-immolative unit. The phenylene ring is optionally further substituted with one, two or three substituents as described herein. In one embodiment, the phenylene group is optionally further substituted with halo, NO₂, R or OR. Preferably n is 0 or 1, most preferably 0.

E is selected such that the group is susceptible to activation, e.g. by light or by the action of an enzyme. E may be —NO₂ or glucoronic acid. The former may be susceptible to the action of a nitroreductase, the latter to the action of a β-glucoronidase.

In this embodiment, the self-immolative linker will allow for release of the protected compound when E is activated, proceeding along the lines shown below (for n=0):

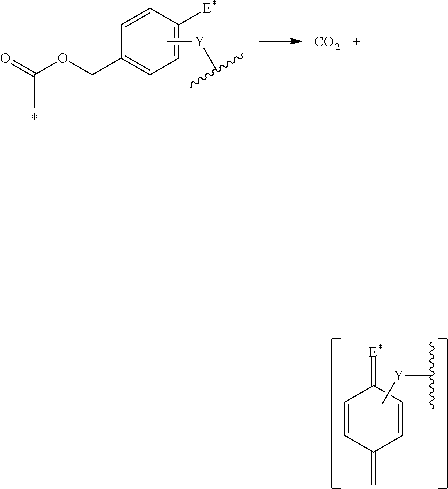

where the asterisk indicates the point of attachment to the N10 position, E* is the activated form of E, and Y is as described above. These groups have the advantage of separating the site of activation from the compound being protected. As described above, the phenylene group may be optionally further substituted.

The group Y may be a covalent bond to L¹.

The group Y may be a functional group selected from:
—C(═O)—
NH—
O—
C(═O)NH—,
—C(═O)O—,
—NHC(═O)—,
—OC(═O)—,
—OC(═O)O—,
—NHC(═O)O—,
—OC(═O)NH—,
—NHC(═O)NH—,
—NHC(═O)NH,
—C(═O)NHC(═O)—, and
—S—.

Where L¹ is a dipeptide, it is preferred that Y is —NH— or —C(═O)—, thereby to form an amide bond between L¹ and Y. In this embodiment, the dipeptide sequence need not be a substrate for an enzymatic activity.

In another embodiment, A is a spacer group. Thus, L¹ and the antibody are indirectly connected.

L¹ and A may be connected by a bond selected from:
—C(═O)NH—,
—C(═O)O—,
—NHC(═O)—,
—OC(═O)—,
—OC(═O)O—,
—NHC(═O)O—,
—OC(═O)NH—, and
—NHC(═O)NH—.

In one embodiment, the group A is:

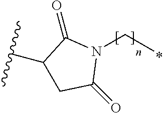

where the asterisk indicates the point of attachment to L¹, the wavy line indicates the point of attachment to the antibody, and n is 0 to 6. In one embodiment, n is 5.

In one embodiment, the group A is:

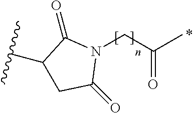

where the asterisk indicates the point of attachment to L¹, the wavy line indicates the point of attachment to the antibody, and n is 0 to 6. In one embodiment, n is 5.

In one embodiment, the group A is:

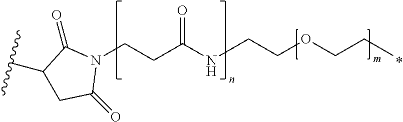

where the asterisk indicates the point of attachment to L¹, the wavy line indicates the point of attachment to the antibody, n is 0 or 1, and m is 0 to 30. In a preferred embodiment, n is 1 and m is 0 to 10, 1 to 8, preferably 4 to 8, and most preferably 4 or 8. In another embodiment, m is 10 to 30, and preferably 20 to 30. Alternatively, m is 0 to 50. In this embodiment, m is preferably 10⁻⁴⁰ and n is 1.

In one embodiment, the group A is:

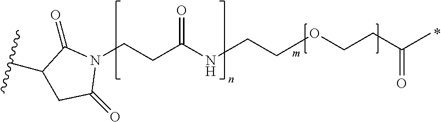

where the asterisk indicates the point of attachment to L¹, the wavy line indicates the point of attachment to the antibody, n is 0 or 1, and m is 0 to 30. In a preferred embodiment, n is 1 and m is 0 to 10, 1 to 8, preferably 4 to 8, and most preferably 4 or 8. In another embodiment, m is 10 to 30, and preferably 20 to 30. Alternatively, m is 0 to 50. In this embodiment, m is preferably 10-40 and n is 1.

In one embodiment, the connection between the antibody and A is through a thiol residue of the antibody and a maleimide group of A.

In one embodiment, the connection between the antibody and A is:

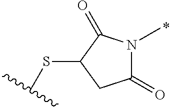 [SMILES: *SC1CC(=O)N(C)C1=O]

where the asterisk indicates the point of attachment to the remaining portion of A and the wavy line indicates the point of attachment to the remaining portion of the antibody. In this embodiment, the S atom is typically derived from the antibody.

In each of the embodiments above, an alternative functionality may be used in place of the maleimide-derived group shown below:

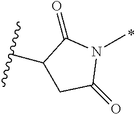 [SMILES: *C1CC(=O)N(C)C1=O]

where the wavy line indicates the point of attachment to the antibody as before, and the asterisk indicates the bond to the remaining portion of the A group.

In one embodiment, the maleimide-derived group is replaced with the group:

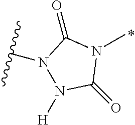 [SMILES: *n1[nH]c(=O)n(C)c1=O]

where the wavy line indicates point of attachment to the antibody, and the asterisk indicates the bond to the remaining portion of the A group.

In one embodiment, the maleimide-derived group is replaced with a group, which optionally together with the antibody, is selected from:
—C(═O)NH—,
—C(═O)O—,
—NHC(═O)—,
—OC(═O)—,
—OC(═O)O—,
—NHC(═O)O—,
—OC(═O)NH—,
—NHC(═O)NH—,
—NHC(═O)NH,
—C(═O)NHC(═O)—,
—S—,
—S—S—,
—CH₂C(═O)—
C(═O)CH₂—,
═N—NH—, and
NH—N═.

In one embodiment, the maleimide-derived group is replaced with a group, which optionally together with the antibody, is selected from:

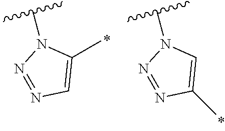

where the wavy line indicates either the point of attachment to the antibody or the bond to the remaining portion of the A group, and the asterisk indicates the other of the point of attachment to the antibody or the bond to the remaining portion of the A group.

Other groups suitable for connecting L¹ to the antibody are described in WO 2005/082023.

In one embodiment, the Connecting Group A is present, the Trigger L¹ is present and Self-Immolative Linker L² is absent. Thus, L¹ and the Drug unit are directly connected via a bond. Equivalently in this embodiment, L² is a bond. This may be particularly relevant when D^{L} is of Formula II.

L¹ and D may be connected by a bond selected from:
—C(═O)N<,
—C(═O)O—,
—NHC(═O)—,
—OC(═O)—,
—NHC(═O)O—,
—OC(═O)N<, and
—NHC(═O)N<,

where N< or O— are part of D.

In one embodiment, L¹ and D are preferably connected by a bond selected from:
—C(═O)N<, and
—NHC(═O)—.

In one embodiment, L¹ comprises a dipeptide and one end of the dipeptide is linked to D. As described above, the amino acids in the dipeptide may be any combination of natural amino acids and non-natural amino acids. In some embodiments, the dipeptide comprises natural amino acids. Where the linker is a cathepsin labile linker, the dipeptide is the site of action for cathepsin-mediated cleavage. The dipeptide then is a recognition site for cathepsin.

In one embodiment, the group —X₁—X₂— in dipeptide, —NH—X₁—X₂—CO—, is selected from:
Phe-Lys-,
Val-Ala-,
Val-Lys-,
Ala-Lys-,
Val-Cit-,
Phe-Cit-,
Leu-Cit-,
Ile-Cit-,
Phe-Arg-, and
Trp-Cit-;

where Cit is citrulline. In such a dipeptide, —NH— is the amino group of X₁, and CO is the carbonyl group of X₂.

Preferably, the group —X₁—X₂— in dipeptide, —NH—X₁—X₂—CO—, is selected from:
Phe-Lys-,
Val-Ala-,
Val-Lys-,
Ala-Lys-, and
Val-Cit-.

Most preferably, the group —X₁—X₂— in dipeptide, —NH—X₁—X₂—CO—, is -Phe-Lys- or -Val-Ala-.

Other dipeptide combinations of interest include:
Gly-Gly-,
Pro-Pro-, and
Val-Glu-.

Other dipeptide combinations may be used, including those described above.

In one embodiment, L¹-D is:

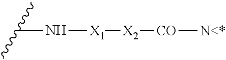

where —NH—X₁—X₂—CO is the dipeptide, —N< is part of the Drug unit, the asterisk indicates the points of attachment to the remainder of the Drug unit, and the wavy line indicates the point of attachment to the remaining portion of L¹ or the point of attachment to A. Preferably, the wavy line indicates the point of attachment to A.

In one embodiment, the dipeptide is valine-alanine and L¹-D is:

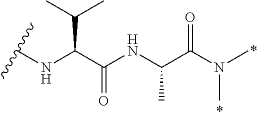 [SMILES: *N[C@H](C(=O)N[C@@H](C)C(=O)N(C)C)C(C)C]

where the asterisks, —N< and the wavy line are as defined above.

In one embodiment, the dipeptide is phenylalnine-lysine and L¹-D is:

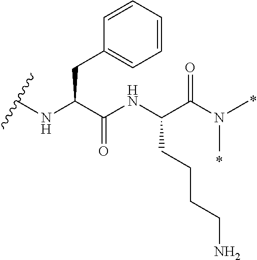 [SMILES: *N[C@@H](Cc1ccccc1)C(=O)N[C@@H](CCCCN)C(=O)N(C)C]

where the asterisks, —N< and the wavy line are as defined above.

In one embodiment, the dipeptide is valine-citrulline.

In one embodiment, the groups A-L¹ are:

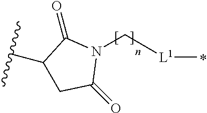

where the asterisk indicates the point of attachment to L² or D, the wavy line indicates the point of attachment to the Ligand unit, and n is 0 to 6. In one embodiment, n is 5.

In one embodiment, the groups A-L¹ are:

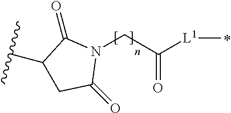

where the asterisk indicates the point of attachment to L² or D, the wavy line indicates the point of attachment to the Ligand unit, and n is 0 to 6. In one embodiment, n is 5.

In one embodiment, the groups A-L¹ are:

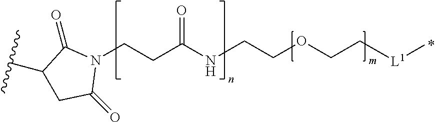

where the asterisk indicates the point of attachment to L² or D, the wavy line indicates the point of attachment to the Ligand unit, n is 0 or 1, and m is 0 to 30. In a preferred embodiment, n is 1 and m is 0 to 10, 1 to 8, preferably 4 to 8, most preferably 4 or 8.

In one embodiment, the groups A-L¹ are:

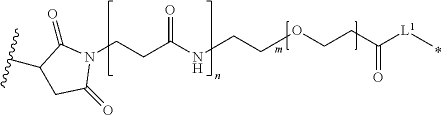

where the asterisk indicates the point of attachment to L² or D, the wavy line indicates the point of attachment to the Ligand unit, n is 0 or 1, and m is 0 to 30. In a preferred embodiment, n is 1 and m is 0 to 10, 1 to 7, preferably 3 to 7, most preferably 3 or 7.

In one embodiment, the groups A-L¹ are:

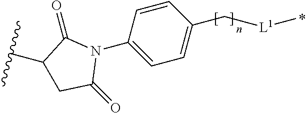

where the asterisk indicates the point of attachment to L² or D, the wavy line indicates the point of attachment to the Ligand unit, and n is 0 to 6. In one embodiment, n is 5.

In one embodiment, the groups A-L¹ are:

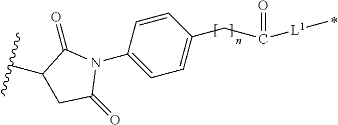

where the asterisk indicates the point of attachment to L² or D, the wavy line indicates the point of attachment to the Ligand unit, and n is 0 to 6. In one embodiment, n is 5.

In one embodiment, the groups A-L¹ are:

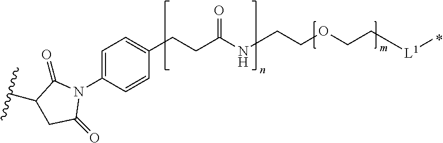

where the asterisk indicates the point of attachment to L² or D, the wavy line indicates the point of attachment to the Ligand unit, n is 0 or 1, and m is 0 to 30. In a preferred embodiment, n is 1 and m is 0 to 10, 1 to 8, preferably 4 to 8, most preferably 4 or 8.

In one embodiment, the groups A-L¹ is:

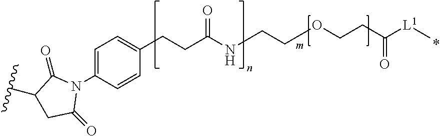

where the asterisk indicates the point of attachment to L² or D, the wavy line indicates the point of attachment to the Ligand unit, n is 0 or 1, and m is 0 to 30. In a preferred embodiment, n is 1 and m is 0 to 10, 1 to 8, preferably 4 to 8, most preferably 4 or 8.

In one embodiment, the groups A-L¹ are:

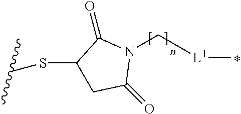

where the asterisk indicates the point of attachment to L² or D, S is a sulfur group of the Ligand unit, the wavy line indicates the point of attachment to the rest of the Ligand unit, and n is 0 to 6. In one embodiment, n is 5.

In one embodiment, the group A-L¹ are:

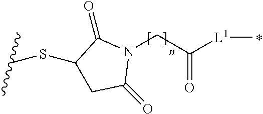

where the asterisk indicates the point of attachment to L² or D, S is a sulfur group of the Ligand unit, the wavy line indicates the point of attachment to the remainder of the Ligand unit, and n is 0 to 6. In one embodiment, n is 5.

In one embodiment, the groups A¹-L¹ are:

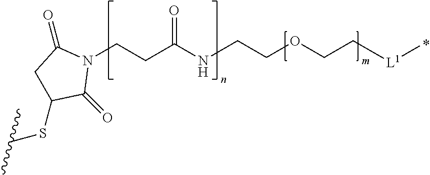

where the asterisk indicates the point of attachment to L² or D, S is a sulfur group of the Ligand unit, the wavy line indicates the point of attachment to the remainder of the Ligand unit, n is 0 or 1, and m is 0 to 30. In a preferred embodiment, n is 1 and m is 0 to 10, 1 to 8, preferably 4 to 8, most preferably 4 or 8.

In one embodiment, the groups A¹-L¹ are:

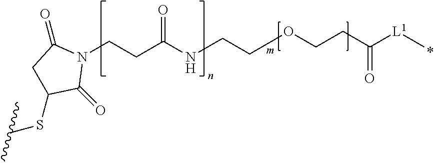

where the asterisk indicates the point of attachment to L² or D, the wavy line indicates the point of attachment to the Ligand unit, n is 0 or 1, and m is 0 to 30. In a preferred embodiment, n is 1 and m is 0 to 10, 1 to 7, preferably 4 to 8, most preferably 4 or 8.

In one embodiment, the groups A¹-L¹ are:

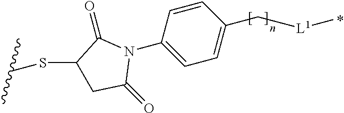

where the asterisk indicates the point of attachment to L² or D, the wavy line indicates the point of attachment to the remainder of the Ligand unit, and n is 0 to 6. In one embodiment, n is 5.

In one embodiment, the groups A¹-L¹ are:

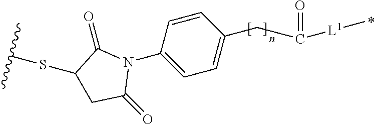

where the asterisk indicates the point of attachment to L² or D, the wavy line indicates the point of attachment to the remainder of the Ligand unit, and n is 0 to 6. In one embodiment, n is 5.

In one embodiment, the groups A¹-L¹ are:

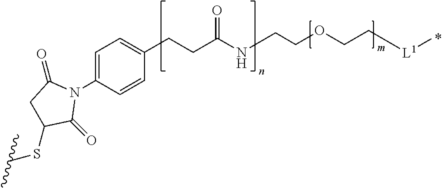

where the asterisk indicates the point of attachment to L² or D, the wavy line indicates the point of attachment to the remainder of the Ligand unit, n is 0 or 1, and m is 0 to 30. In a preferred embodiment, n is 1 and m is 0 to 10, 1 to 8, preferably 4 to 8, most preferably 4 or 8.

In one embodiment, the groups A¹-L¹ are:

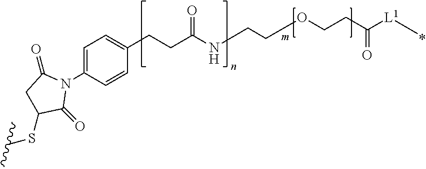

where the asterisk indicates the point of attachment to L² or D, the wavy line indicates the point of attachment to the remainder of the Ligand unit, n is 0 or 1, and m is 0 to 30. In a preferred embodiment, n is 1 and m is 0 to 10, 1 to 8, preferably 4 to 8, most preferably 4 or 8.

The group R^{L′} is derivable from the group R^{L}. The group R^{L} may be converted to a group R^{L′}by connection of an antibody to a functional group of R^{L}. Other steps may be taken to convert R^{L} to R^{L′}. These steps may include the removal of protecting groups, where present, or the installation of an appropriate functional group.

R^{L}

Linkers can include protease-cleavable peptidic moieties comprising one or more amino acid units. Peptide linker reagents may be prepared by solid phase or liquid phase synthesis methods (E. Schröder and K. Lübke, The Peptides, volume 1, pp 76-136 (1965) Academic Press) that are well known in the field of peptide chemistry, including t-BOC chemistry (Geiser et al “Automation of solid-phase peptide synthesis” in Macromolecular Sequencing and Synthesis, Alan R. Liss, Inc., 1988, pp. 199-218) and Fmoc/HBTU chemistry (Fields, G. and Noble, R. (1990) “Solid phase peptide synthesis utilizing 9-fluoroenylmethoxycarbonyl amino acids”, Int. J. Peptide Protein Res. 35:161-214), on an automated synthesizer such as the Rainin Symphony Peptide Synthesizer (Protein Technologies, Inc., Tucson, Ariz.), or Model 433 (Applied Biosystems, Foster City, Calif.).

Exemplary amino acid linkers include a dipeptide, a tripeptide, a tetrapeptide or a pentapeptide. Exemplary dipeptides include: valine-citrulline (vc or val-cit), alanine-phenylalanine (af or ala-phe). Exemplary tripeptides include: glycine-valine-citrulline (gly-val-cit) and glycine-glycine-glycine (gly-gly-gly). Amino acid residues which comprise an amino acid linker component include those occurring naturally, as well as minor amino acids and non-naturally occurring amino acid analogs, such as citrulline. Amino acid linker components can be designed and optimized in their selectivity for enzymatic cleavage by a particular enzymes, for example, a tumor-associated protease, cathepsin B, C and D, or a plasmin protease.

Amino acid side chains include those occurring naturally, as well as minor amino acids and non-naturally occurring amino acid analogs, such as citrulline. Amino acid side chains include hydrogen, methyl, isopropyl, isobutyl, sec-butyl, benzyl, p-hydroxybenzyl, —CH₂OH, —CH(OH)CH₃, —CH₂CH₂SCH₃, —CH₂CONH₂, —CH₂COOH, —CH₂CH₂CONH₂, —CH₂CH₂COOH, —(CH₂)₃NHC(═NH)NH₂, —(CH₂)₃NH₂, —(CH₂)₃NHCOCH₃, —(CH₂)₃NHCHO, —(CH₂)₄NHC(═NH)NH₂, —(CH₂)₄NH₂, —(CH₂)₄NHCOCH₃, —(CH₂)₄NHCHO, —(CH₂)₃NHCONH₂, —(CH₂)₄NHCONH₂, —CH₂CH₂CH(OH)CH₂NH₂, 2-pyridylmethyl-, 3-pyridylmethyl-, 4-pyridylmethyl-, phenyl, cyclohexyl, as well as the following structures:

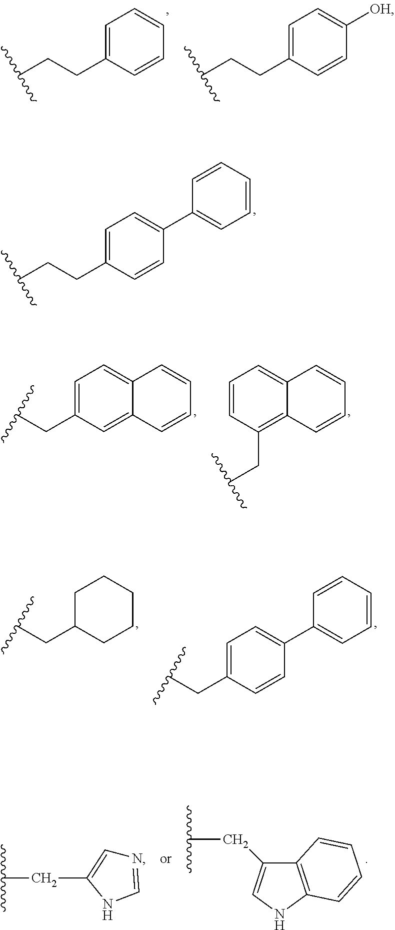

When the amino acid side chains include other than hydrogen (glycine), the carbon atom to which the amino acid side chain is attached is chiral. Each carbon atom to which the amino acid side chain is attached is independently in the (S) or (R) configuration, or a racemic mixture. Drug-linker reagents may thus be enantiomerically pure, racemic, or diastereomeric.

In exemplary embodiments, amino acid side chains are selected from those of natural and non-natural amino acids, including alanine, 2-amino-2-cyclohexylacetic acid, 2-amino-2-phenylacetic acid, arginine, asparagine, aspartic acid, cysteine, glutamine, glutamic acid, glycine, histidine, isoleucine, leucine, lysine, methionine, norleucine, phenylalanine, proline, serine, threonine, tryptophan, tyrosine, valine, γ-aminobutyric acid, α,α-dimethyl γ-aminobutyric acid, β,β-dimethyl γ-aminobutyric acid, ornithine, and citrulline (Cit).

An exemplary valine-citrulline (val-cit or vc) dipeptide linker reagent useful for constructing a linker-PBD drug moiety intermediate for conjugation to an antibody, having a para-aminobenzylcarbamoyl (PAB) self-immolative spacer has the structure:

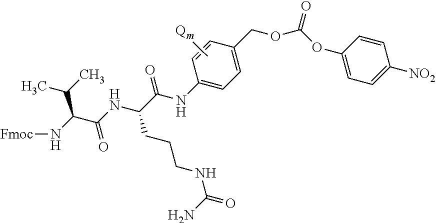

where Q is C₁-C₅ alkyl, —O—(C₁-C₈ alkyl), -halogen, —NO₂ or —CN; and m is an integer ranging from 0-4.

An exemplary phe-lys(Mtr) dipeptide linker reagent having a p-aminobenzyl group can be prepared according to Dubowchik, et al. (1997) Tetrahedron Letters, 38:5257-60, and has the structure:

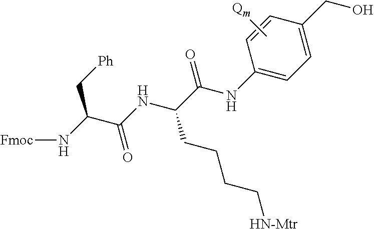

where Mtr is mono-4-methoxytrityl, Q is C₁-C₈ alkyl, —O—(C₁-C₈ alkyl), -halogen, —NO₂ or —CN; and m is an integer ranging from 0-4.

The “self-immolative linker” PAB (para-aminobenzyloxycarbonyl), attaches the drug moiety to the antibody in the antibody drug conjugate (Carl et al (1981) J. Med. Chem. 24:479-480; Chakravarty et al (1983) J. Med. Chem. 26:638-644; U.S. Pat. No. 6,214,345; US20030130189; US20030096743; U.S. Pat. No. 6,759,509; US20040052793; U.S. Pat. No. 6,218,519; U.S. Pat. No. 6,835,807; U.S. Pat. No. 6,268,488; US20040018194; WO98/13059; US20040052793; U.S. Pat. No. 6,677,435; U.S. Pat. No. 5,621,002; US20040121940; WO2004/032828). Other examples of self-immolative spacers besides PAB include, but are not limited to: (i) aromatic compounds that are electronically similar to the PAB group such as 2-aminoimidazol-5-methanol derivatives (Hay et al. (1999) Bioorg. Med. Chem. Lett. 9:2237), thiazoles (U.S. Pat. No. 7,375,078), multiple, elongated PAB units (de Groot et al (2001) J. Org. Chem. 66:8815-8830); and ortho or para-aminobenzylacetals; and (ii) homologated styryl PAB analogs (U.S. Pat. No. 7,223,837). Spacers can be used that undergo cyclization upon amide bond hydrolysis, such as substituted and unsubstituted 4-aminobutyric acid amides (Rodrigues et al (1995) Chemistry Biology 2:223), appropriately substituted bicyclo[2.2.1] and bicyclo[2.2.2] ring systems (Storm et al (1972) J. Amer. Chem. Soc. 94:5815) and 2-aminophenylpropionic acid amides (Amsberry, et al (1990) J. Org. Chem. 55:5867). Elimination of amine-containing drugs that are substituted at glycine (Kingsbury et al (1984) J. Med. Chem. 27:1447) are also examples of self-immolative spacers useful in ADC.

In one embodiment, a valine-citrulline dipeptide PAB analog reagent has a 2,6 dimethyl phenyl group and has the structure:

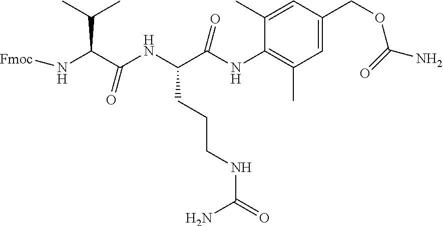

Linker reagents useful for the antibody drug conjugates of the disclosure include, but are not limited to: BMPEO, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate), and bis-maleimide reagents: DTME, BMB, BMDB, BMH, BMOE, 1,8-bis-maleimidodiethyleneglycol (BM(PEO)₂), and 1,11-bis-maleimidotriethyleneglycol (BM(PEO)₃), which are commercially available from Pierce Biotechnology, Inc., ThermoScientific, Rockford, Ill., and other reagent suppliers. Bis-maleimide reagents allow the attachment of a free thiol group of a cysteine residue of an antibody to a thiol-containing drug moiety, label, or linker intermediate, in a sequential or concurrent fashion. Other functional groups besides maleimide, which are reactive with a thiol group of an antibody, PBD drug moiety, or linker intermediate include iodoacetamide, bromoacetamide, vinyl pyridine, disulfide, pyridyl disulfide, isocyanate, and isothiocyanate.

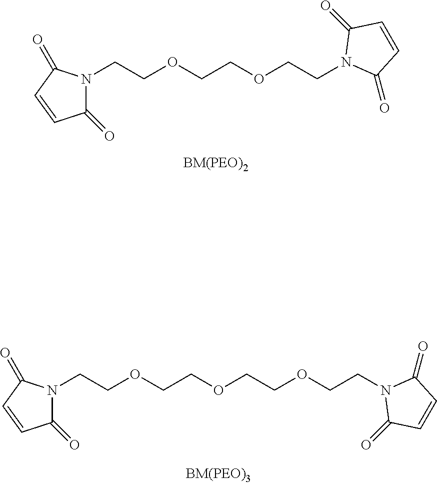 [SMILES: O=C1C=CC(=O)N1CCOCCOCCN1C(=O)C=CC1=O ; O=C1C=CC(=O)N1CCOCCOCCOCCN1C(=O)C=CC1=O]

Other embodiments of linker reagents are: N-succinimidyl-4-(2-pyridylthio)pentanoate (SPP), N-succinimidyl-3-(2-pyridyldithio) propionate (SPDP, Carlsson et al (1978) Biochem. J. 173:723-737), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Useful linker reagents can also be obtained via other commercial sources, such as Molecular Biosciences Inc. (Boulder, Colo.), or synthesized in accordance with procedures described in Toki et al (2002) J. Org. Chem. 67:1866-1872; U.S. Pat. No. 6,214,345; WO 02/088172; US 2003130189; US2003096743; WO 03/026577; WO 03/043583; and WO 04/032828.

The Linker may be a dendritic type linker for covalent attachment of more than one drug moiety through a branching, multifunctional linker moiety to an antibody (US 2006/116422; US 2005/271615; de Groot et al (2003) Angew. Chem. Int. Ed. 42:4490-4494; Amir et al (2003) Angew. Chem. Int. Ed. 42:4494-4499; Shamis et al (2004) J. Am. Chem. Soc. 126:1726-1731; Sun et al (2002) Bioorganic & Medicinal Chemistry Letters 12:2213-2215; Sun et al (2003) Bioorganic & Medicinal Chemistry 11:1761-1768; King et al (2002) Tetrahedron Letters 43:1987-1990). Dendritic linkers can increase the molar ratio of drug to antibody, i.e. loading, which is related to the potency of the ADC. Thus, where an antibody bears only one reactive cysteine thiol group, a multitude of drug moieties may be attached through a dendritic or branched linker.

One exemplary embodiment of a dendritic type linker has the structure:

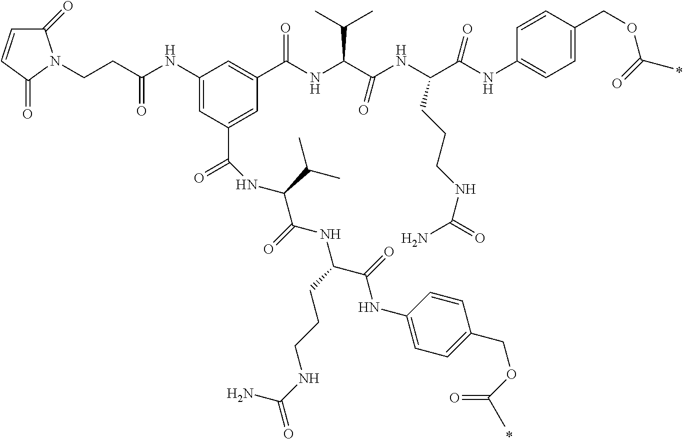

where the asterisk indicate the point of attachment to the N10 position of a PBD moiety.

R^{C}, Capping Group

The conjugate of the first aspect of the disclosure may have a capping group R^{C} at the N10 position.

The group R^{C} is removable from the N10 position of the PBD moiety to leave an N10-C11 imine bond, a carbinolamine, a substituted carbinolamine, where QR¹¹ is OSO₃M, a bisulfite adduct, a thiocarbinolamine, a substituted thiocarbinolamine, or a substituted carbinalamine.

In one embodiment, R^{C}, may be a protecting group that is removable to leave an N10-C11 imine bond, a carbinolamine, a substituted cabinolamine, or, where QR¹¹ is OSO₃M, a bisulfite adduct. In one embodiment, R^{C} is a protecting group that is removable to leave an N10-C11 imine bond.

The group R^{C} is intended to be removable under the same conditions as those required for the removal of the group R¹⁰, for example to yield an N10-C11 imine bond, a carbinolamine and so on. The capping group acts as a protecting group for the intended functionality at the N10 position. The capping group is intended not to be reactive towards an antibody. For example, R^{C} is not the same as R^{L}.

Compounds having a capping group may be used as intermediates in the synthesis of dimers having an imine monomer. Alternatively, compounds having a capping group may be used as conjugates, where the capping group is removed at the target location to yield an imine, a carbinolamine, a substituted cabinolamine and so on. Thus, in this embodiment, the capping group may be referred to as a therapeutically removable nitrogen protecting group, as defined in the inventors' earlier application WO 00/12507.

In one embodiment, the group R^{C} is removable under the conditions that cleave the linker R^{L} of the group R¹⁰. Thus, in one embodiment, the capping group is cleavable by the action of an enzyme.

In an alternative embodiment, the capping group is removable prior to the connection of the linker R^{L} to the antibody. In this embodiment, the capping group is removable under conditions that do not cleave the linker R^{L}.

Where a compound includes a functional group G¹ to form a connection to the antibody, the capping group is removable prior to the addition or unmasking of G¹.

The capping group may be used as part of a protecting group strategy to ensure that only one of the monomer units in a dimer is connected to an antibody.

The capping group may be used as a mask for a N10-C11 imine bond. The capping group may be removed at such time as the imine functionality is required in the compound. The capping group is also a mask for a carbinolamine, a substituted cabinolamine, and a bisulfite adduct, as described above.

R^{C} may be an N10 protecting group, such as those groups described in the inventors' earlier application, WO 00/12507. In one embodiment, R^{C} is a therapeutically removable nitrogen protecting group, as defined in the inventors' earlier application, WO 00/12507.

In one embodiment, R^{C} is a carbamate protecting group.

In one embodiment, the carbamate protecting group is selected from:
Alloc, Fmoc, Boc, Troc, Teoc, Psec, Cbz and PNZ.

Optionally, the carbamate protecting group is further selected from Moc.

In one embodiment, R^{C} is a linker group R^{L} lacking the functional group for connection to the antibody.

This application is particularly concerned with those R^{C} groups which are carbamates.

In one embodiment, R^{C} is a group:

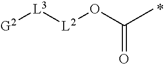

where the asterisk indicates the point of attachment to the N10 position, G² is a terminating group, L³ is a covalent bond or a cleavable linker L¹, L² is a covalent bond or together with OC(═O) forms a self-immolative linker.

Where L³ and L² are both covalent bonds, G² and OC(═O) together form a carbamate protecting group as defined above.

L¹ is as defined above in relation to R¹⁰.

L² is as defined above in relation to R¹⁰.

Various terminating groups are described below, including those based on well known protecting groups.

In one embodiment L³ is a cleavable linker L¹, and L², together with OC(═O), forms a self-immolative linker. In this embodiment, G² is Ac (acetyl) or Moc, or a carbamate protecting group selected from:
Alloc, Fmoc, Boc, Troc, Teoc, Psec, Cbz and PNZ.

Optionally, the carbamate protecting group is further selected from Moc.

In another embodiment, G² is an acyl group —C(═O)G³, where G³ is selected from alkyl (including cycloalkyl, alkenyl and alkynyl), heteroalkyl, heterocyclyl and aryl (including heteroaryl and carboaryl). These groups may be optionally substituted. The acyl group together with an amino group of L³ or L², where appropriate, may form an amide bond. The acyl group together with a hydroxy group of L³ or L², where appropriate, may form an ester bond.

In one embodiment, G³ is heteroalkyl. The heteroalkyl group may comprise polyethylene glycol. The heteroalkyl group may have a heteroatom, such as O or N, adjacent to the acyl group, thereby forming a carbamate or carbonate group, where appropriate, with a heteroatom present in the group L³ or L², where appropriate.

In one embodiment, G³ is selected from NH₂, NHR and NRR′. Preferably, G³ is NRR′.

In one embodiment G² is the group:

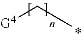

where the asterisk indicates the point of attachment to L³, n is 0 to 6 and G⁴ is selected from OH, OR, SH, SR, COOR, CONH₂, CONHR, CONRR′, NH₂, NHR, NRR′, NO₂, and halo. The groups OH, SH, NH₂ and NHR are protected. In one embodiment, n is 1 to 6, and preferably n is 5. In one embodiment, G⁴ is OR, SR, COOR, CONH₂, CONHR, CONRR′, and NRR′. In one embodiment, G⁴ is OR, SR, and NRR′. Preferably G⁴ is selected from OR and NRR′, most preferably G⁴ is OR. Most preferably G⁴ is OMe.

In one embodiment, the group G² is:

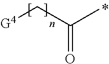

where the asterisk indicates the point of attachment to L³, and n and G⁴ are as defined above.

In one embodiment, the group G² is:

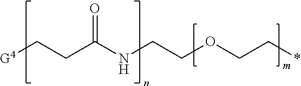

where the asterisk indicates the point of attachment to L³, n is 0 or 1, m is 0 to 50, and G⁴ is selected from OH, OR, SH, SR, COOR, CONH₂, CONHR, CONRR′, NH₂, NHR, NRR′, NO₂, and halo. In a preferred embodiment, n is 1 and m is 0 to 10, 1 to 2, preferably 4 to 8, and most preferably 4 or 8. In another embodiment, n is 1 and m is 10 to 50, preferably 20 to 40. The groups OH, SH, NH₂ and NHR are protected. In one embodiment, G⁴ is OR, SR, COOR, CONH₂, CONHR, CONRR′, and NRR′. In one embodiment, G⁴ is OR, SR, and NRR′. Preferably G⁴ is selected from OR and NRR′, most preferably G⁴ is OR.

Preferably G⁴ is OMe.

In one embodiment, the group G² is:

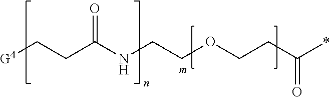

where the asterisk indicates the point of attachment to L³, and n, m and G⁴ are as defined above.

In one embodiment, the group G² is:

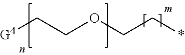

where n is 1-20, m is 0-6, and G⁴ is selected from OH, OR, SH, SR, COOR, CONH₂, CONHR, CONRR′, NH₂, NHR, NRR′, NO₂, and halo. In one embodiment, n is 1-10. In another embodiment, n is 10 to 50, preferably 20 to 40. In one embodiment, n is 1. In one embodiment, m is 1. The groups OH, SH, NH₂ and NHR are protected. In one embodiment, G⁴ is OR, SR, COOR, CONH₂, CONHR, CONRR′, and NRR′. In one embodiment, G⁴ is OR, SR, and NRR′. Preferably G⁴ is selected from OR and NRR′, most preferably G⁴ is OR.

Preferably G⁴ is OMe.

In one embodiment, the group G² is:

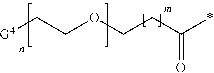

where the asterisk indicates the point of attachment to L³, and n, m and G⁴ are as defined above.

In each of the embodiments above G⁴ may be OH, SH, NH₂ and NHR. These groups are preferably protected.

In one embodiment, OH is protected with Bzl, TBDMS, or TBDPS.

In one embodiment, SH is protected with Acm, Bzl, Bzl-OMe, Bzl-Me, or Trt.

In one embodiment, NH₂ or NHR are protected with Boc, Moc, Z—Cl, Fmoc, Z, or Alloc.

In one embodiment, the group G² is present in combination with a group L³, which group is a dipeptide.

The capping group is not intended for connection to the antibody. Thus, the other monomer present in the dimer serves as the point of connection to the antibody via a linker.

Accordingly, it is preferred that the functionality present in the capping group is not available for reaction with an antibody. Thus, reactive functional groups such as OH, SH, NH₂, COOH are preferably avoided. However, such functionality may be present in the capping group if protected, as described above.

### Embodiments

Embodiments of the present disclosure include ConjA wherein the antibody is as defined above.

Embodiments of the present disclosure include ConjB wherein the antibody is as defined above.

Embodiments of the present disclosure include ConjC wherein the antibody is as defined above.

Embodiments of the present disclosure include ConjD wherein the antibody is as defined above.

Embodiments of the present disclosure include ConjE wherein the antibody is as defined above.

Embodiments of the present disclosure include ConjF wherein the antibody is as defined above.

Embodiments of the present disclosure include ConjG wherein the antibody is as defined above.

Embodiments of the present disclosure include ConjH wherein the antibody is as defined above.

Drug Loading

The drug loading is the average number of PBD drugs per antibody, e.g. antibody. Where the compounds of the disclosure are bound to native cysteines, drug loading may range from 1 to 8 drugs (D^{L}) per antibody, i.e. where 1, 2, 3, 4, 5, 6, 7, and 8 drug moieties are covalently attached to the antibody. Compositions of conjgates include collections of antibodies, conjugated with a range of drugs, from 1 to 8. Where the compounds of the disclosure are bound to lysines, drug loading may range from 1 to 80 drugs (D^{L}) per antibody, although an upper limit of 40, 20, 10 or 8 may be preferred. Compositions of conjugates include collections of antibodies, conjugated with a range of drugs, from 1 to 80, 1 to 40, 1 to 20, 1 to 10 or 1 to 8.

The average number of drugs per antibody in preparations of ADC from conjugation reactions may be characterized by conventional means such as UV, reverse phase HPLC, HIC, mass spectroscopy, ELISA assay, and electrophoresis. The quantitative distribution of ADC in terms of p may also be determined. By ELISA, the averaged value of p in a particular preparation of ADC may be determined (Hamblett et al (2004) Clin. Cancer Res. 10:7063-7070; Sanderson et al (2005) Clin. Cancer Res. 11:843-852). However, the distribution of p (drug) values is not discernible by the antibody-antigen binding and detection limitation of ELISA. Also, ELISA assay for detection of antibody-drug conjugates does not determine where the drug moieties are attached to the antibody, such as the heavy chain or light chain fragments, or the particular amino acid residues. In some instances, separation, purification, and characterization of homogeneous ADC where p is a certain value from ADC with other drug loadings may be achieved by means such as reverse phase HPLC or electrophoresis. Such techniques are also applicable to other types of conjugates.

For some antibody-drug conjugates, p may be limited by the number of attachment sites on the antibody. For example, an antibody may have only one or several cysteine thiol groups, or may have only one or several sufficiently reactive thiol groups through which a linker may be attached. Higher drug loading, e.g. p>5, may cause aggregation, insolubility, toxicity, or loss of cellular permeability of certain antibody-drug conjugates.

Typically, fewer than the theoretical maximum of drug moieties are conjugated to an antibody during a conjugation reaction. An antibody may contain, for example, many lysine residues that do not react with the drug-linker intermediate (D-L) or linker reagent. Only the most reactive lysine groups may react with an amine-reactive linker reagent. Also, only the most reactive cysteine thiol groups may react with a thiol-reactive linker reagent. Generally, antibodies do not contain many, if any, free and reactive cysteine thiol groups which may be linked to a drug moiety. Most cysteine thiol residues in the antibodies of the compounds exist as disulfide bridges and must be reduced with a reducing agent such as dithiothreitol (DTT) or TCEP, under partial or total reducing conditions. The loading (drug/antibody ratio) of an ADC may be controlled in several different manners, including: (i) limiting the molar excess of drug-linker intermediate (D-L) or linker reagent relative to antibody, (ii) limiting the conjugation reaction time or temperature, and (iii) partial or limiting reductive conditions for cysteine thiol modification.

Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by engineering one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

Cysteine amino acids may be engineered at reactive sites in an antibody and which do not form intrachain or intermolecular disulfide linkages (Junutula, et al., 2008b Nature Biotech., 26(8):925-932; Dornan et al (2009) Blood 114(13):2721-2729; U.S. Pat. No. 7,521,541; U.S. Pat. No. 7,723,485; WO2009/052249). The engineered cysteine thiols may react with linker reagents or the drug-linker reagents of the present disclosure which have thiol-reactive, electrophilic groups such as maleimide or alpha-halo amides to form ADC with cysteine engineered antibodies and the PBD drug moieties. The location of the drug moiety can thus be designed, controlled, and known. The drug loading can be controlled since the engineered cysteine thiol groups typically react with thiol-reactive linker reagents or drug-linker reagents in high yield. Engineering an IgG antibody to introduce a cysteine amino acid by substitution at a single site on the heavy or light chain gives two new cysteines on the symmetrical antibody. A drug loading near 2 can be achieved with near homogeneity of the conjugation product ADC.

Alternatively, site-specific conjugation can be achieved by engineering antibodies to contain unnatural amino acids in their heavy and/or light chains as described by Axup et al. ((2012), Proc Natl Acad Sci USA. 109(40):16101-16116). The unnatural amino acids provide the additional advantage that orthogonal chemistry can be designed to attach the linker reagent and drug.

Where more than one nucleophilic or electrophilic group of the antibody reacts with a drug-linker intermediate, or linker reagent followed by drug moiety reagent, then the resulting product is a mixture of ADC compounds with a distribution of drug moieties attached to an antibody, e.g. 1, 2, 3, etc. Liquid chromatography methods such as polymeric reverse phase (PLRP) and hydrophobic interaction (HIC) may separate compounds in the mixture by drug loading value. Preparations of ADC with a single drug loading value (p) may be isolated, however, these single loading value ADCs may still be heterogeneous mixtures because the drug moieties may be attached, via the linker, at different sites on the antibody.

Thus the antibody-drug conjugate compositions of the disclosure include mixtures of antibody-drug conjugate compounds where the antibody has one or more PBD drug moieties and where the drug moieties may be attached to the antibody at various amino acid residues.

In one embodiment, the average number of dimer pyrrolobenzodiazepine groups per antibody is in the range 1 to 20. In some embodiments the range is selected from 1 to 8, 2 to 8, 2 to 6, 2 to 4, and 4 to 8.

In some embodiments, there is one dimer pyrrolobenzodiazepine group per antibody.

Includes Other Forms

Unless otherwise specified, included in the above are the well known ionic, salt, solvate, and protected forms of these substituents. For example, a reference to carboxylic acid (—COOH) also includes the anionic (carboxylate) form (—COO⁻), a salt or solvate thereof, as well as conventional protected forms. Similarly, a reference to an amino group includes the protonated form (—N⁺HR¹R²), a salt or solvate of the amino group, for example, a hydrochloride salt, as well as conventional protected forms of an amino group. Similarly, a reference to a hydroxyl group also includes the anionic form (—O⁻), a salt or solvate thereof, as well as conventional protected forms.

Salts

It may be convenient or desirable to prepare, purify, and/or handle a corresponding salt of the active compound, for example, a pharmaceutically-acceptable salt. Examples of pharmaceutically acceptable salts are discussed in Berge, et al., J. Pharm. Sci, 66, 1-19 (1977).

For example, if the compound is anionic, or has a functional group which may be anionic (e.g. —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e. NH₄⁺) and substituted ammonium ions (e.g. NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g. —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, trifluoroacetic acid and valeric. Examples of suitable polymeric organic anions include, but are not limited to, those derived from the following polymeric acids: tannic acid, carboxymethyl cellulose.

Solvates

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

The disclosure includes compounds where a solvent adds across the imine bond of the PBD moiety, which is illustrated below where the solvent is water or an alcohol (R^{A}OH, where R^{A} is C₁₋₄ alkyl):

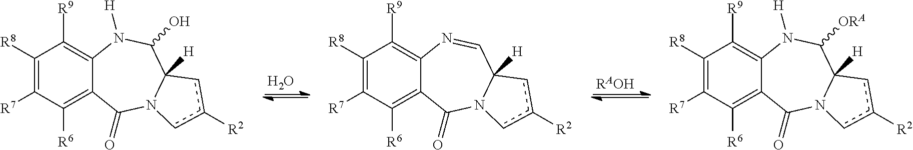

These forms can be called the carbinolamine and carbinolamine ether forms of the PBD (as described in the section relating to R¹⁰ above). The balance of these equilibria depend on the conditions in which the compounds are found, as well as the nature of the moiety itself.

These particular compounds may be isolated in solid form, for example, by lyophilisation.

Isomers

Certain compounds of the disclosure may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and I-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. The compounds of the disclosure may contain asymmetric or chiral centers, and therefore exist in different stereoisomeric forms. It is intended that all stereoisomeric forms of the compounds of the disclosure, including but not limited to, diastereomers, enantiomers and atropisomers, as well as mixtures thereof such as racemic mixtures, form part of the present disclosure. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or l meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g. C₁₋₇ alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

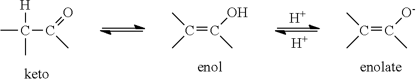

The term “tautomer” or “tautomeric form” refers to structural isomers of different energies which are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Examples of isotopes that can be incorporated into compounds of the disclosure include isotopes of hydrogen, carbon, nitrogen, oxygen, phosphorous, fluorine, and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl, and ¹²⁵I. Various isotopically labeled compounds of the present disclosure, for example those into which radioactive isotopes such as 3H, 13C, and 14C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays, or in radioactive treatment of patients. Deuterium labelled or substituted therapeutic compounds of the disclosure may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life or reduced dosage requirements. An 18F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. Further, substitution with heavier isotopes, particularly deuterium (i.e., 2H or D) may afford certain therapeutic advantages resulting from greater metabolic stability, for example increased in vivo half-life or reduced dosage requirements or an improvement in therapeutic index. It is understood that deuterium in this context is regarded as a substituent. The concentration of such a heavier isotope, specifically deuterium, may be defined by an isotopic enrichment factor. In the compounds of this disclosure any atom not specifically designated as a particular isotope is meant to represent any stable isotope of that atom.

Unless otherwise specified, a reference to a particular compound includes all such isomeric forms, including (wholly or partially) racemic and other mixtures thereof. Methods for the preparation (e.g. asymmetric synthesis) and separation (e.g. fractional crystallisation and chromatographic means) of such isomeric forms are either known in the art or are readily obtained by adapting the methods taught herein, or known methods, in a known manner.

### Biological Activity

### In Vitro Cell Proliferation Assays

Generally, the cytotoxic or cytostatic activity of an antibody-drug conjugate (ADC) is measured by: exposing mammalian cells having receptor proteins to the antibody of the ADC in a cell culture medium; culturing the cells for a period from about 6 hours to about 5 to 7 days; and measuring cell viability. Cell-based in vitro assays are used to measure viability (proliferation), cytotoxicity, and induction of apoptosis (caspase activation) of an ADC of the disclosure.

The in vitro potency of antibody-drug conjugates can be measured by a cell proliferation assay. The CellTiter-Glo® Luminescent Cell Viability Assay is a commercially available (Promega Corp., Madison, Wis.), homogeneous assay method based on the recombinant expression of Coleoptera luciferase (U.S. Pat. Nos. 5,583,024; 5,674,713 and 5,700,670). This cell proliferation assay determines the number of viable cells in culture based on quantitation of the ATP present, an indicator of metabolically active cells (Crouch et al (1993) J. Immunol. Meth. 160:81-88; U.S. Pat. No. 6,602,677). The CellTiter-Glo® Assay is conducted in 96 well format, making it amenable to automated high-throughput screening (HTS) (Cree et al (1995) AntiCancer Drugs 6:398-404). The homogeneous assay procedure involves adding the single reagent (CellTiter-Glo® Reagent) directly to cells cultured in serum-supplemented medium. Cell washing, removal of medium and multiple pipetting steps are not required. The system detects as few as 15 cells/well in a 384-well format in 10 minutes after adding reagent and mixing. The cells may be treated continuously with ADC, or they may be treated and separated from ADC. Generally, cells treated briefly, i.e. 3 hours, showed the same potency effects as continuously treated cells.

The homogeneous “add-mix-measure” format results in cell lysis and generation of a luminescent signal proportional to the amount of ATP present. The amount of ATP is directly proportional to the number of cells present in culture. The CellTiter-Glo® Assay generates a “glow-type” luminescent signal, produced by the luciferase reaction, which has a half-life generally greater than five hours, depending on cell type and medium used. Viable cells are reflected in relative luminescence units (RLU). The substrate, Beetle Luciferin, is oxidatively decarboxylated by recombinant firefly luciferase with concomitant conversion of ATP to AMP and generation of photons.

The in vitro potency of antibody-drug conjugates can also be measured by a cytotoxicity assay. Cultured adherent cells are washed with PBS, detached with trypsin, diluted in complete medium, containing 10% FCS, centrifuged, re-suspended in fresh medium and counted with a haemocytometer. Suspension cultures are counted directly. Monodisperse cell suspensions suitable for counting may require agitation of the suspension by repeated aspiration to break up cell clumps.

The cell suspension is diluted to the desired seeding density and dispensed (100 μl per well) into black 96 well plates. Plates of adherent cell lines are incubated overnight to allow adherence. Suspension cell cultures can be used on the day of seeding.

A stock solution (1 ml) of ADC (20 μg/ml) is made in the appropriate cell culture medium. Serial 10-fold dilutions of stock ADC are made in 15 ml centrifuge tubes by serially transferring 100 μl to 900 μl of cell culture medium.

Four replicate wells of each ADC dilution (100 μl) are dispensed in 96-well black plates, previously plated with cell suspension (100 μl), resulting in a final volume of 200 μl. Control wells receive cell culture medium (100 μl).

If the doubling time of the cell line is greater than 30 hours, ADC incubation is for 5 days, otherwise a four day incubation is done.

At the end of the incubation period, cell viability is assessed with the Alamar blue assay. AlamarBlue (Invitrogen) is dispensed over the whole plate (20 μl per well) and incubated for 4 hours. Alamar blue fluorescence is measured at excitation 570 nm, emission 585 nm on the Varioskan flash plate reader. Percentage cell survival is calculated from the mean fluorescence in the ADC treated wells compared to the mean fluorescence in the control wells.

Use

The conjugates of the disclosure may be used to provide a PBD compound at a target location.

The target location is preferably a proliferative cell population. The antibody is an antibody for an antigen present on a proliferative cell population.

In one embodiment the antigen is absent or present at a reduced level in a non-proliferative cell population compared to the amount of antigen present in the proliferative cell population, for example a tumour cell population.

At the target location the linker may be cleaved so as to release a compound RelA, RelB, RelC, RelD, RelE or RelG. Thus, the conjugate may be used to selectively provide a compound RelA, RelB, RelC, RelD, RelE or RelG to the target location.

The linker may be cleaved by an enzyme present at the target location.

The target location may be in vitro, in vivo or ex vivo.

The antibody-drug conjugate (ADC) compounds of the disclosure include those with utility for anticancer activity. In particular, the compounds include an antibody conjugated, i.e. covalently attached by a linker, to a PBD drug moiety, i.e. toxin. When the drug is not conjugated to an antibody, the PBD drug has a cytotoxic effect. The biological activity of the PBD drug moiety is thus modulated by conjugation to an antibody. The antibody-drug conjugates (ADC) of the disclosure selectively deliver an effective dose of a cytotoxic agent to tumour tissue whereby greater selectivity, i.e. a lower efficacious dose, may be achieved.

Thus, in one aspect, the present disclosure provides a conjugate compound as described herein for use in therapy.

In a further aspect there is also provides a conjugate compound as described herein for use in the treatment of a proliferative disease. A second aspect of the present disclosure provides the use of a conjugate compound in the manufacture of a medicament for treating a proliferative disease.

One of ordinary skill in the art is readily able to determine whether or not a candidate conjugate treats a proliferative condition for any particular cell type. For example, assays which may conveniently be used to assess the activity offered by a particular compound are described in the examples below.

The term “proliferative disease” pertains to an unwanted or uncontrolled cellular proliferation of excessive or abnormal cells which is undesired, such as, neoplastic or hyperplastic growth, whether in vitro or in vivo.

Examples of proliferative conditions include, but are not limited to, benign, pre-malignant, and malignant cellular proliferation, including but not limited to, neoplasms and tumours (e.g. histocytoma, glioma, astrocyoma, osteoma), cancers (e.g. lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreas cancer, brain cancer, sarcoma, osteosarcoma, Kaposi's sarcoma, melanoma), lymphomas, leukemias, psoriasis, bone diseases, fibroproliferative disorders (e.g. of connective tissues), and atherosclerosis. Cancers of particular interest include, but are not limited to, metastatic cancer cells, such as circulating tumour cells, which may be found circulating in body fluids such as blood or lymph, lymphomas (e.g., non-Hodgkin's lymphoma, NHL), leukemia (particularly acute myeloid leukemia, AML) and ovarian cancers. Any type of cell may be treated, including but not limited to, lung, gastrointestinal (including, e.g. bowel, colon), breast (mammary), ovarian, prostate, liver (hepatic), kidney (renal), bladder, pancreas, brain, and skin.

It is contemplated that the antibody-drug conjugates (ADC) of the present disclosure may be used to treat various diseases or disorders, e.g. characterized by the overexpression of a tumour antigen. Exemplary conditions or hyperproliferative disorders include benign or malignant tumours; leukemias, haematological, and lymphoid malignancies. Others include neuronal, glial, astrocytal, hypothalamic, glandular, macrophagal, epithelial, stromal, blastocoelic, inflammatory, angiogenic and immunologic, including autoimmune, disorders.

Generally, the disease or disorder to be treated is a hyperproliferative disease such as cancer. Examples of cancer to be treated herein include, but are not limited to, carcinoma, lymphoma, blastoma, sarcoma, and leukemias or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

Autoimmune diseases for which the ADC compounds may be used in treatment include rheumatologic disorders (such as, for example, rheumatoid arthritis, Sjögren's syndrome, scleroderma, lupus such as SLE and lupus nephritis, polymyositis/dermatomyositis, cryoglobulinemia, anti-phospholipid antibody syndrome, and psoriatic arthritis), osteoarthritis, autoimmune gastrointestinal and liver disorders (such as, for example, inflammatory bowel diseases (e.g. ulcerative colitis and Crohn's disease), autoimmune gastritis and pernicious anemia, autoimmune hepatitis, primary biliary cirrhosis, primary sclerosing cholangitis, and celiac disease), vasculitis (such as, for example, ANCA-associated vasculitis, including Churg-Strauss vasculitis, Wegener's granulomatosis, and polyarteriitis), autoimmune neurological disorders (such as, for example, multiple sclerosis, opsoclonus myoclonus syndrome, myasthenia gravis, neuromyelitis optica, Parkinson's disease, Alzheimer's disease, and autoimmune polyneuropathies), renal disorders (such as, for example, glomerulonephritis, Goodpasture's syndrome, and Berger's disease), autoimmune dermatologic disorders (such as, for example, psoriasis, urticaria, hives, pemphigus vulgaris, bullous pemphigoid, and cutaneous lupus erythematosus), hematologic disorders (such as, for example, thrombocytopenic purpura, thrombotic thrombocytopenic purpura, post-transfusion purpura, and autoimmune hemolytic anemia), atherosclerosis, uveitis, autoimmune hearing diseases (such as, for example, inner ear disease and hearing loss), Behcet's disease, Raynaud's syndrome, organ transplant, and autoimmune endocrine disorders (such as, for example, diabetic-related autoimmune diseases such as insulin-dependent diabetes mellitus (IDDM), Addison's disease, and autoimmune thyroid disease (e.g. Graves' disease and thyroiditis)). More preferred such diseases include, for example, rheumatoid arthritis, ulcerative colitis, ANCA-associated vasculitis, lupus, multiple sclerosis, Sjögren's syndrome, Graves' disease, IDDM, pernicious anemia, thyroiditis, and glomerulonephritis.

Methods of Treatment

The conjugates of the present disclosure may be used in a method of therapy. Also provided is a method of treatment, comprising administering to a subject in need of treatment a therapeutically-effective amount of a conjugate compound of the disclosure. The term “therapeutically effective amount” is an amount sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors.

A compound of the disclosure may be administered alone or in combination with other treatments, either simultaneously or sequentially dependent upon the condition to be treated. Examples of treatments and therapies include, but are not limited to, chemotherapy (the administration of active agents, including, e.g. drugs, such as chemotherapeutics); surgery; and radiation therapy.

A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy.

Examples of chemotherapeutic agents include: erlotinib (TARCEVA®, Genentech/OSI Pharm.), docetaxel (TAXOTERE®, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum(II), CAS No. 15663-27-1), carboplatin (CAS No. 41575-94-4), paclitaxel (TAXOL®, Bristol-Myers Squibb Oncology, Princeton, N.J.), trastuzumab (HERCEPTIN®, Genentech), temozolomide (4-methyl-5-oxo-2,3,4,6,8-pentazabicyclo[4.3.0]nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl)phenoxy]-N,N-dimethylethanamine, NOLVADEX®, ISTUBAL®, VALODEX®), and doxorubicin (ADRIAMYCIN®), Akti-1/2, HPPD, and rapamycin.

More examples of chemotherapeutic agents include: oxaliplatin (ELOXATIN@, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (PI3K inhibitor, Semafore Pharmaceuticals), BEZ-235 (PI3K inhibitor, Novartis), XL-147 (PI3K inhibitor, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERB®, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, II), vandetanib (rINN, ZD6474, ZACTIMA®, AstraZeneca), chloranmbucil, AG1478, AG1571 (SU 5271; Sugen), temsirolimus (TORISEL@, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g. calicheamicin, calicheamicin gammal I, calicheamicin omegall (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, nemorubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; capecitabine (XELODA®, Roche); ibandronate; CPT-11; topoisomerase inhibitor RFS 2000; difluoromethylornithine (DMFO); retinoids such as retinoic acid; and pharmaceutically acceptable salts, acids and derivatives of any of the above.

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON@ (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR@(vorozole), FEMARA@ (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as MEK inhibitors (WO 2007/044515); (v) lipid kinase inhibitors; (vi) antisense oligonucleotides, particularly those which inhibit expression of genes in signaling pathways implicated in aberrant cell proliferation, for example, PKC-alpha, Raf and H-Ras, such as oblimersen (GENASENSE®, Genta Inc.); (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN@rlL-2; topoisomerase 1 inhibitors such as LURTOTECAN®; ABARELIX® rmRH; (ix) anti-angiogenic agents such as bevacizumab (AVASTIN®, Genentech); and pharmaceutically acceptable salts, acids and derivatives of any of the above.

Also included in the definition of “chemotherapeutic agent” are therapeutic antibodies such as alemtuzumab (Campath), bevacizumab (AVASTIN@, Genentech); cetuximab (ERBITUX®, Imclone); panitumumab (VECTIBIX®, Amgen), rituximab (RITUXAN®, Genentech/Biogen Idec), ofatumumab (ARZERRA®, GSK), pertuzumab (PERJETA™, OMNITARG™, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth).

Humanized monoclonal antibodies with therapeutic potential as chemotherapeutic agents in combination with the conjugates of the disclosure include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

Pharmaceutical compositions according to the present disclosure, and for use in accordance with the present disclosure, may comprise, in addition to the active ingredient, i.e. a conjugate compound, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

The disclosure provides methods relating to the identification of subjects particularly suitable for treatment with the conjugate or pharmaceutical composition of the disclosure. Also provided are methods for determining the optimum timing and dosage of administration of the antibodies of the disclosure to a subject. In some embodiments the subject has a proliferative disease, such as cancer. In some embodiments the subject has an autoimmune disease. Preferably, administration of the treatment inhibits or reduces one or more aspects of the disease, for example reduces tumour volume, or reduces the level of one or more biomarkers of tumour progression, such as AXL, Akt3, or GAS6. In some embodiments the level of the biomarker is reduced to no more than 90% of the level immediately before treatment, such as no more than 80%, no more than 70%, no more than 60%, no more than 50%, no more than 40%, no more than 30%, no more than 20%, no more than 10%, or no more than 5% of the level immediately before treatment.

In one aspect the disclosure provides a method of selecting a subject for treatment with the conjugate or pharmaceutical composition of the disclosure, the method comprising assessing the level of one or more biomarkers associated with disease pathology, wherein subjects having the one or more biomarker, or subjects having a level of the one or more biomarkers which exceeds a threshold level, are selected for treatment. In some embodiments the biomarker is AXL, Akt3, or GAS6. In some embodiments the threshold is at least 10% higher than the upper boundary of the normal clinical range, such as at least 20% higher, at least 30% higher, at least 40% higher, at least 50% higher, at least 100% higher, or at least 200% higher.

In another aspect the disclosure provides a method of timing the administration of treatment of a subject with the conjugate or pharmaceutical composition of the disclosure, the method comprising assessing the level of one or more biomarkers associated with disease pathology, wherein the treatment is administered when the subject has the one or more biomarker, or the subject has a level of one or more biomarkers which exceeds a threshold level. In some embodiments the biomarker is AXL, Akt3, or GAS6. In some embodiments the threshold is at least 10% higher than the upper boundary of the normal clinical range, such as at least 20% higher, at least 30% higher, at least 40% higher, at least 50% higher, at least 100% higher, or at least 200% higher.

In another aspect the disclosure provides a method of determining the optimum dosage of the conjugate or pharmaceutical composition of the disclosure for administration to a subject, the method comprising assessing the level of one or more biomarkers associated with disease pathology, wherein subjects having the one or more biomarker, or subjects having a level of the one or more biomarkers which exceeds the threshold level, are selected for a particular dosage level. In some embodiments the biomarker is AXL, Akt3, or GAS6. In some embodiments the threshold is at least 10% higher than the upper boundary of the normal clinical range, such as at least 20% higher, at least 30% higher, at least 40% higher, at least 50% higher, at least 100% higher, or at least 200% higher.

In some embodiments the level of one or more biomarkers is assessed in a sample of blood, urine, other body fluid, or tissue. Level of one or more biomarkers samples can be assessed by immunoassay, proteomic assay, nucleic acid hybridization or amplification assays, immunohistochemistry, or in situ hybridization assays.

Formulations

While it is possible for the conjugate compound to be used (e.g., administered) alone, it is often preferable to present it as a composition or formulation.

In one embodiment, the composition is a pharmaceutical composition (e.g., formulation, preparation, medicament) comprising a conjugate compound, as described herein, and a pharmaceutically acceptable carrier, diluent, or excipient.

In one embodiment, the composition is a pharmaceutical composition comprising at least one conjugate compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents.

In one embodiment, the composition further comprises other active agents, for example, other therapeutic or prophylactic agents.

Suitable carriers, diluents, excipients, etc. can be found in standard pharmaceutical texts. See, for example, Handbook of Pharmaceutical Additives, 2nd Edition (eds. M. Ash and I. Ash), 2001 (Synapse Information Resources, Inc., Endicott, N.Y., USA), Remington's Pharmaceutical Sciences, 20th edition, pub. Lippincott, Williams & Wilkins, 2000; and Handbook of Pharmaceutical Excipients, 2nd edition, 1994.

Another aspect of the present disclosure pertains to methods of making a pharmaceutical composition comprising admixing at least one [¹¹C]-radiolabelled conjugate or conjugate-like compound, as defined herein, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the active compound.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

The formulations may be prepared by any methods well known in the art of pharmacy. Such methods include the step of bringing into association the active compound with a carrier which constitutes one or more accessory ingredients. In general, the formulations are prepared by uniformly and intimately bringing into association the active compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations suitable for parenteral administration (e.g., by injection), include aqueous or non-aqueous, isotonic, pyrogen-free, sterile liquids (e.g., solutions, suspensions), in which the active ingredient is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the active ingredient in the liquid is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

Dosage

It will be appreciated by one of skill in the art that appropriate dosages of the conjugate compound, and compositions comprising the conjugate compound, can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the active compound is in the range of about 100 ng to about 25 mg (more typically about 1 pg to about 10 mg) per kilogram body weight of the subject per day. Where the active compound is a salt, an ester, an amide, a prodrug, or the like, the amount administered is calculated on the basis of the parent compound and so the actual weight to be used is increased proportionately.

In one embodiment, the active compound is administered to a human patient according to the following dosage regime: about 100 mg, 3 times daily.

In one embodiment, the active compound is administered to a human patient according to the following dosage regime: about 150 mg, 2 times daily.

In one embodiment, the active compound is administered to a human patient according to the following dosage regime: about 200 mg, 2 times daily.

However in one embodiment, the conjugate compound is administered to a human patient according to the following dosage regime: about 50 or about 75 mg, 3 or 4 times daily.

In one embodiment, the conjugate compound is administered to a human patient according to the following dosage regime: about 100 or about 125 mg, 2 times daily.

The dosage amounts described above may apply to the conjugate (including the PBD moiety and the linker to the antibody) or to the effective amount of PBD compound provided, for example the amount of compound that is releasable after cleavage of the linker.

For the prevention or treatment of disease, the appropriate dosage of an ADC of the disclosure will depend on the type of disease to be treated, as defined above, the severity and course of the disease, whether the molecule is administered for preventive or therapeutic purposes, previous therapy, the patient's clinical history and response to the antibody, and the discretion of the attending physician. The molecule is suitably administered to the patient at one time or over a series of treatments. Depending on the type and severity of the disease, about 1 μg/kg to 15 mg/kg (e.g. 0.1-20 mg/kg) of molecule is an initial candidate dosage for administration to the patient, whether, for example, by one or more separate administrations, or by continuous infusion. A typical daily dosage might range from about 1 μg/kg to 100 mg/kg or more, depending on the factors mentioned above. An exemplary dosage of ADC to be administered to a patient is in the range of about 0.1 to about 10 mg/kg of patient weight. For repeated administrations over several days or longer, depending on the condition, the treatment is sustained until a desired suppression of disease symptoms occurs. An exemplary dosing regimen comprises a course of administering an initial loading dose of about 4 mg/kg, followed by additional doses every week, two weeks, or three weeks of an ADC. Other dosage regimens may be useful. The progress of this therapy is easily monitored by conventional techniques and assays.

Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically-effective amount,” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

Similarly, the term “prophylactically-effective amount,” as used herein, pertains to that amount of an active compound, or a material, composition or dosage from comprising an active compound, which is effective for producing some desired prophylactic effect, commensurate with a reasonable benefit/risk ratio, when administered in accordance with a desired treatment regimen.

Preparation of Drug Conjugates

Antibody drug conjugates may be prepared by several routes, employing organic chemistry reactions, conditions, and reagents known to those skilled in the art, including reaction of a nucleophilic group of an antibody with a drug-linker reagent. This method may be employed to prepare the antibody-drug conjugates of the disclosure.

Nucleophilic groups on antibodies include, but are not limited to side chain thiol groups, e.g. cysteine. Thiol groups are nucleophilic and capable of reacting to form covalent bonds with electrophilic groups on linker moieties such as those of the present disclosure. Certain antibodies have reducible interchain disulfides, i.e. cysteine bridges. Antibodies may be made reactive for conjugation with linker reagents by treatment with a reducing agent such as DTT (Cleland's reagent, dithiothreitol) or TCEP (tris(2-carboxyethyl)phosphine hydrochloride; Getz et al (1999) Anal. Biochem. Vol 273:73-80; Soltec Ventures, Beverly, Mass.). Each cysteine disulfide bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol.

The Subject/Patient

The subject/patient may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject/patient may be any of its forms of development, for example, a foetus. In one preferred embodiment, the subject/patient is a human.

Further Preferences

The following preferences may apply to all aspects of the disclosure as described above, or may relate to a single aspect. The preferences may be combined together in any combination.

In some embodiments, R^{6′}, R^{7′}, R^{9′}, and Y′ are preferably the same as R⁶, R⁷, R⁹, and Y respectively.

Dimer Link

Y and Y′ are preferably O.

R″ is preferably a C₃₋₇ alkylene group with no substituents. More preferably R″ is a C₃, C₅ or C₇ alkylene. Most preferably, R″ is a C₃ or C₅ alkylene.

R⁶ to R⁹

R⁹ is preferably H.

R⁶ is preferably selected from H, OH, OR, SH, NH₂, nitro and halo, and is more preferably H or halo, and most preferably is H.

R⁷ is preferably selected from H, OH, OR, SH, SR, NH₂, NHR, NRR′, and halo, and more preferably independently selected from H, OH and OR, where R is preferably selected from optionally substituted C₁₋₇ alkyl, C₃₋₁₀ heterocyclyl and C₅₋₁₀ aryl groups. R may be more preferably a C₁₋₄ alkyl group, which may or may not be substituted. A substituent of interest is a C₅₋₆ aryl group (e.g. phenyl). Particularly preferred substituents at the 7-positions are OMe and OCH₂Ph. Other substituents of particular interest are dimethylamino (i.e. —NMe₂); —(OC₂H4)_{q}OMe, where q is from 0 to 2; nitrogen-containing C₆ heterocyclyls, including morpholino, piperidinyl and N-methyl-piperazinyl.

These preferences apply to R^{9′}, R^{6′} and R^{7′} respectively.

R¹²

When there is a double bond present between C2′ and C3′, R¹² is selected from:

(a) C₅₋₁₀ aryl group, optionally substituted by one or more substituents selected from the group comprising: halo, nitro, cyano, ether, C₁₋₇ alkyl, C₃₋₇ heterocyclyl and bis-oxy-C₁₋₃ alkylene;

(b) C₁₋₅ saturated aliphatic alkyl;

(c) C₃₋₆ saturated cycloalkyl;

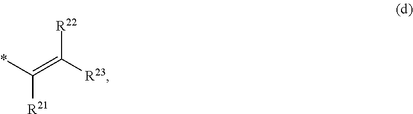

wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5;

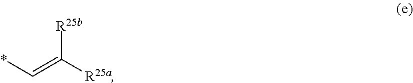

wherein one of R^{25a} and R^{25b} is H and the other is selected from: phenyl, which phenyl is optionally substituted by a group selected from halo methyl, methoxy; pyridyl; and thiophenyl; and

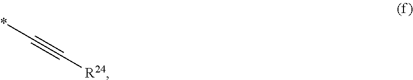

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo methyl, methoxy; pyridyl; and thiophenyl.

When R¹² is a C₅₋₁₀ aryl group, it may be a C₅₋₇ aryl group. A C₅₋₇ aryl group may be a phenyl group or a C₅₋₇ heteroaryl group, for example furanyl, thiophenyl and pyridyl. In some embodiments, R¹² is preferably phenyl. In other embodiments, R¹² is preferably thiophenyl, for example, thiophen-2-yl and thiophen-3-yl.

When R¹² is a C₅₋₁₀ aryl group, it may be a C₈₋₁₀ aryl, for example a quinolinyl or isoquinolinyl group. The quinolinyl or isoquinolinyl group may be bound to the PBD core through any available ring position. For example, the quinolinyl may be quinolin-2-yl, quinolin-3-yl, quinolin-4yl, quinolin-5-yl, quinolin-6-yl, quinolin-7-yl and quinolin-8-yl. Of these quinolin-3-yl and quinolin-6-yl may be preferred. The isoquinolinyl may be isoquinolin-1-yl, isoquinolin-3-yl, isoquinolin-4yl, isoquinolin-5-yl, isoquinolin-6-yl, isoquinolin-7-yl and isoquinolin-8-yl. Of these isoquinolin-3-yl and isoquinolin-6-yl may be preferred.

When R¹² is a C₅₋₁₀ aryl group, it may bear any number of substituent groups. It preferably bears from 1 to 3 substituent groups, with 1 and 2 being more preferred, and singly substituted groups being most preferred. The substituents may be any position.

Where R¹² is C₅₋₇ aryl group, a single substituent is preferably on a ring atom that is not adjacent the bond to the remainder of the compound, i.e. it is preferably β or γ to the bond to the remainder of the compound. Therefore, where the C₅₋₇ aryl group is phenyl, the substituent is preferably in the meta- or para-positions, and more preferably is in the para-position.

Where R¹² is a C₈₋₁₀ aryl group, for example quinolinyl or isoquinolinyl, it may bear any number of substituents at any position of the quinoline or isoquinoline rings. In some embodiments, it bears one, two or three substituents, and these may be on either the proximal and distal rings or both (if more than one substituent).

R¹² Substituents, when R¹² is a C₅₋₁₀ Aryl Group

If a substituent on R¹² when R¹² is a C₅₋₁₀ aryl group is halo, it is preferably F or Cl, more preferably Cl.

If a substituent on R¹² when R¹² is a 5-10 aryl group is ether, it may in some embodiments be an alkoxy group, for example, a C₁₋₇ alkoxy group (e.g. methoxy, ethoxy) or it may in some embodiments be a C₅₋₇ aryloxy group (e.g phenoxy, pyridyloxy, furanyloxy). The alkoxy group may itself be further substituted, for example by an amino group (e.g. dimethylamino).

If a substituent on R¹² when R¹² is a C₅₋₁₀ aryl group is C1-7 alkyl, it may preferably be a C₁₋₄ alkyl group (e.g. methyl, ethyl, propryl, butyl).

If a substituent on R¹² when R¹² is a C₅₋₁₀ aryl group is C₃₋₇ heterocyclyl, it may in some embodiments be C₆ nitrogen containing heterocyclyl group, e.g. morpholino, thiomorpholino, piperidinyl, piperazinyl. These groups may be bound to the rest of the PBD moiety via the nitrogen atom. These groups may be further substituted, for example, by C₁₋₄ alkyl groups.

If the C₆ nitrogen containing heterocyclyl group is piperazinyl, the said further substituent may be on the second nitrogen ring atom.

If a substituent on R¹² when R¹² is a C₅₋₁₀ aryl group is bis-oxy-C₁₋₃ alkylene, this is preferably bis-oxy-methylene or bis-oxy-ethylene.

If a substituent on R¹² when R¹² is a C₅₋₁₀ aryl group is ester, this is preferably methyl ester or ethyl ester.

Particularly preferred substituents when R¹² is a C₅₋₁₀ aryl group include methoxy, ethoxy, fluoro, chloro, cyano, bis-oxy-methylene, methyl-piperazinyl, morpholino and methyl-thiophenyl. Other particularly preferred substituent for R¹² are dimethylaminopropyloxy and carboxy.

Particularly preferred substituted R¹² groups when R¹² is a C₅₋₁₀ aryl group include, but are not limited to, 4-methoxy-phenyl, 3-methoxyphenyl, 4-ethoxy-phenyl, 3-ethoxy-phenyl, 4-fluoro-phenyl, 4-chloro-phenyl, 3,4-bisoxymethylene-phenyl, 4-methylthiophenyl, 4-cyanophenyl, 4-phenoxyphenyl, quinolin-3-yl and quinolin-6-yl, isoquinolin-3-yl and isoquinolin-6-yl, 2-thienyl, 2-furanyl, methoxynaphthyl, and naphthyl. Another possible substituted R¹² group is 4-nitrophenyl. R¹² groups of particular interest include 4-(4-methylpiperazin-1-yl)phenyl and 3,4-bisoxymethylene-phenyl.

When R¹² is C₁₋₅ saturated aliphatic alkyl, it may be methyl, ethyl, propyl, butyl or pentyl. In some embodiments, it may be methyl, ethyl or propyl (n-pentyl or isopropyl). In some of these embodiments, it may be methyl. In other embodiments, it may be butyl or pentyl, which may be linear or branched.

When R¹² is C₃₋₆ saturated cycloalkyl, it may be cyclopropyl, cyclobutyl, cyclopentyl or cyclohexyl. In some embodiments, it may be cyclopropyl.

When R¹² is

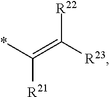

each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5. In some embodiments, the total number of carbon atoms in the R¹² group is no more than 4 or no more than 3.

In some embodiments, one of R²¹, R²² and R²³ is H, with the other two groups being selected from H, C₁₋₃ saturated alkyl, C₂₋₃ alkenyl, C₂₋₃ alkynyl and cyclopropyl.

In other embodiments, two of R²¹, R²² and R²³ are H, with the other group being selected from H, C₁₋₃ saturated alkyl, C₂₋₃ alkenyl, C₂₋₃ alkynyl and cyclopropyl.

In some embodiments, the groups that are not H are selected from methyl and ethyl. In some of these embodiments, the groups that re not H are methyl.

In some embodiments, R²¹ is H.

In some embodiments, R²² is H.

In some embodiments, R²³ is H.

In some embodiments, R²¹ and R²² are H.

In some embodiments, R²¹ and R²³ are H.

In some embodiments, R²² and R²³ are H.

An R¹² group of particular interest is:

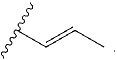 [SMILES: */C=C/C]

When R¹² is

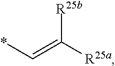

one of R^{25a} and R^{25b} is H and the other is selected from: phenyl, which phenyl is optionally substituted by a group selected from halo, methyl, methoxy; pyridyl; and thiophenyl. In some embodiments, the group which is not H is optionally substituted phenyl. If the phenyl optional substituent is halo, it is preferably fluoro. In some embodiment, the phenyl group is unsubstituted.

When R¹² is

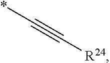

R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo methyl, methoxy; pyridyl; and thiophenyl. If the phenyl optional substituent is halo, it is preferably fluoro. In some embodiment, the phenyl group is unsubstituted.

In some embodiments, R²⁴ is selected from H, methyl, ethyl, ethenyl and ethynyl. In some of these embodiments, R²⁴ is selected from H and methyl.

When there is a single bond present between C2′ and C3′,

R¹² is

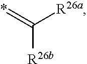

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester.

In some embodiments, it is preferred that R^{26a} and R^{26b} are both H.

In other embodiments, it is preferred that R^{26a} and R^{26b} are both methyl.

In further embodiments, it is preferred that one of R^{26a} and R^{26b} is H, and the other is selected from C₁₋₄ saturated alkyl, C₂₋₃ alkenyl, which alkyl and alkenyl groups are optionally substituted. In these further embodiment, it may be further preferred that the group which is not H is selected from methyl and ethyl.

R²

The above preferences for R¹² apply equally to R².

R²²

In some embodiments, R²² is of formula IIa.

A in R²² when it is of formula IIa may be phenyl group or a C₅₋₇ heteroaryl group, for example furanyl, thiophenyl and pyridyl. In some embodiments, A is preferably phenyl.

Q²—X may be on any of the available ring atoms of the C₅₋₇ aryl group, but is preferably on a ring atom that is not adjacent the bond to the remainder of the compound, i.e. it is preferably β or γ to the bond to the remainder of the compound. Therefore, where the C₅₋₇ aryl group (A) is phenyl, the substituent (Q²-X) is preferably in the meta- or para-positions, and more preferably is in the para-position.

In some embodiments, Q¹ is a single bond. In these embodiments, Q² is selected from a single bond and —Z—(CH₂)ₙ—, where Z is selected from a single bond, O, S and NH and is from 1 to 3. In some of these embodiments, Q² is a single bond. In other embodiments, Q² is —Z—(CH₂)ₙ—. In these embodiments, Z may be O or S and n may be 1 or n may be 2. In other of these embodiments, Z may be a single bond and n may be 1.

In other embodiments, Q¹ is —CH═CH—.

In other embodiments, R²² is of formula IIb. In these embodiments, R^{C1}, R^{C2} and R^{C3} are independently selected from H and unsubstituted C₁₋₂ alkyl. In some preferred embodiments, R^{C1}, R^{C2} and R^{C3} are all H. In other embodiments, R^{C1}, R^{C2} and R^{C3} are all methyl. In certain embodiments, R^{C1}, R^{C2} and R^{C3} are independently selected from H and methyl.

X is a group selected from the list comprising: O—R^{L2′}, S—R^{L2′}, CO₂—R^{L2′}, CO—R^{L2′}, NH—C(═O)—R^{L2}′, NHNH—R^{L2′}, CONHNH—R^{L2′},

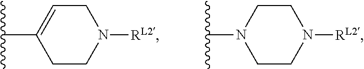

R^{N}L^{2′}, wherein R^{N} is selected from the group comprising H and C₁₋₄ alkyl. X may preferably be: OH, SH, CO₂H, —N═C═O or NHR^{N}, and may more preferably be: O—R^{L2′}, S—R^{L2′}, CO₂—R^{L2′}, —NH—C(═O)—R^{L2′} or NH—R^{L2′}. Particularly preferred groups include: O—R^{L2′}, S—R^{L2′} and NH—R^{L2′}, with NH—R^{L2′} being the most preferred group.

In some embodiments R²² is of formula IIc. In these embodiments, it is preferred that Q is NR^{N}—R^{L2′}. In other embodiments, Q is O—R^{L2′}. In further embodiments, Q is S—R^{L2′}. R^{N} is preferably selected from H and methyl. In some embodiment, R^{N} is H. In other embodiments, R^{N} is methyl.

In some embodiments, R²² may be -A-CH₂—X and -A-X. In these embodiments, X may be O—R^{L2′}, S—R^{L2′}, CO₂—R^{L2′}, CO—R^{L2′} and NH—R^{L2′}. In particularly preferred embodiments, X may be NHR^{L2′}.

R¹⁰, R¹¹

In some embodiments, R¹⁰ and R¹¹ together form a double bond between the nitrogen and carbon atoms to which they are bound.

In some embodiments, R¹¹ is OH.

In some embodiments, R¹¹ is OMe.

In some embodiments, R¹¹ is SO_{z}M, where z is 2 or 3 and M is a monovalent pharmaceutically acceptable cation.

R^{11a}

In some embodiments, R^{11a} is OH.

In some embodiments, R^{11a} is OMe.

In some embodiments, R^{11a} is SO_{z}M, where z is 2 or 3 and M is a monovalent pharmaceutically acceptable cation.

R²⁰, R²¹

In some embodiments, R²⁰ and R²¹ together form a double bond between the nitrogen and carbon atoms to which they are bound.

In some embodiments R²⁰ is H.

In some embodiments, R²⁰ is R^{C}.

In some embodiments, R²¹ is OH.

In some embodiments, R²¹ is OMe.

In some embodiments, R²¹ is SO_{z}M, where z is 2 or 3 and M is a monovalent pharmaceutically acceptable cation.

R³⁰, R³¹

In some embodiments, R³⁰ and R³¹ together form a double bond between the nitrogen and carbon atoms to which they are bound.

In some embodiments, R³¹ is OH.

In some embodiments, R³¹ is OMe.

In some embodiments, R³¹ is SO_{z}M, where z is 2 or 3 and M is a monovalent pharmaceutically acceptable cation.

M and z

It is preferred that M is a monovalent pharmaceutically acceptable cation, and is more preferably Na⁺.

z is preferably 3.

Preferred conjugates of the first aspect of the present disclosure may have a D^{L} of formula Ia:

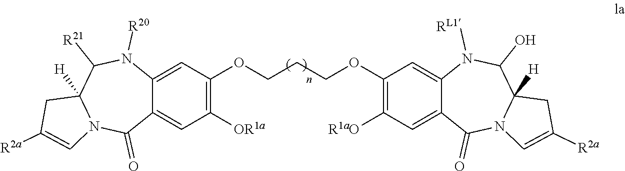

where

R^{L1′}, R²⁰ and R²¹ are as defined above;

n is 1 or 3;

R^{1a} is methyl or phenyl; and

R^{2a} is selected from:

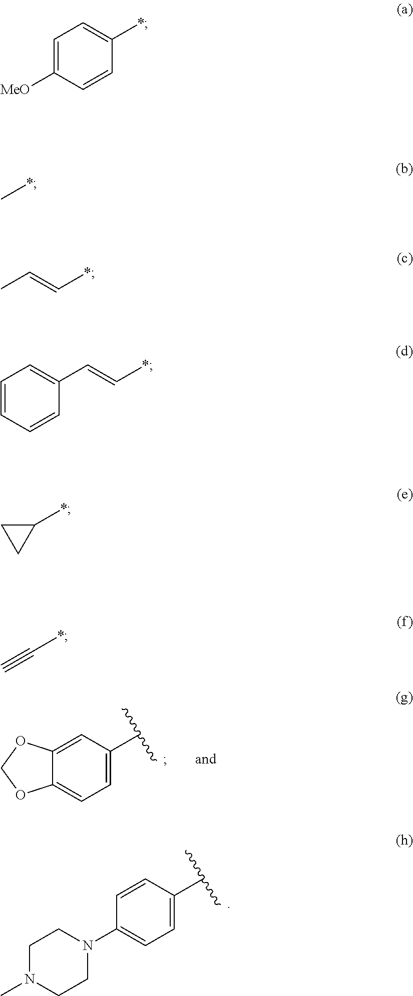

Preferred conjugates of the first aspect of the present disclosure may have a D^{L} of formula Ib:

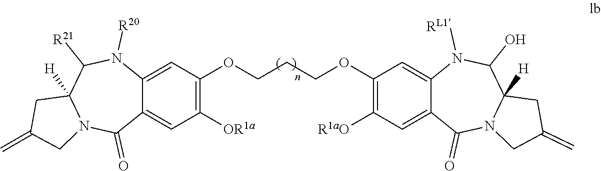

where

R^{L1′}, R²⁰ and R²¹ are as defined above;

n is 1 or 3; and

R^{1a} is methyl or phenyl.

Preferred conjugates of the first aspect of the present disclosure may have a D^{L} of formula Ic:

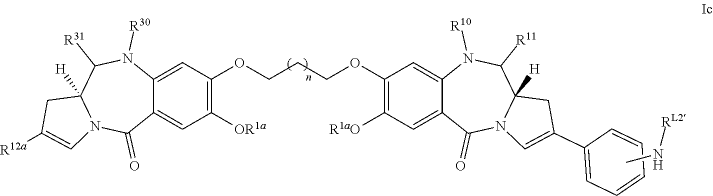

where R^{L2′}, R¹⁰, R¹¹, R³⁰ and R³¹ are as defined above

n is 1 or 3;

R^{12a} is selected from:

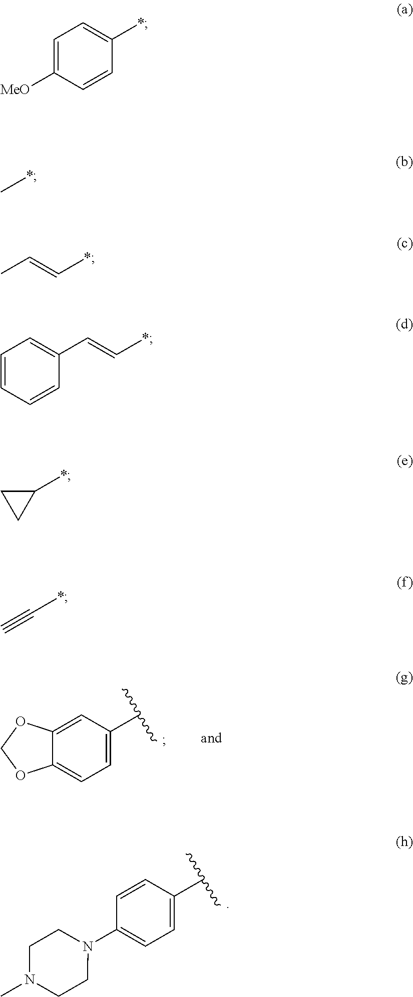

the amino group is at either the meta or para positions of the phenyl group.

Preferred conjugates of the first aspect of the present disclosure may have a D^{L} of formula Id:

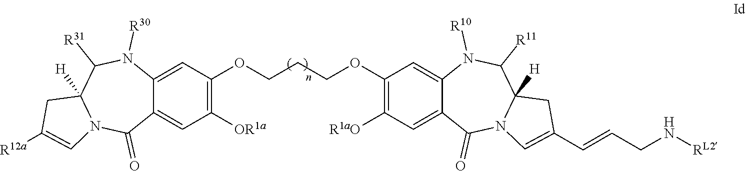

where R^{L2′}, R¹⁰, R¹, R³⁰ and R³¹ are as defined above

n is 1 or 3;

R^{1a} is methyl or phenyl;

R^{12a} is selected from:

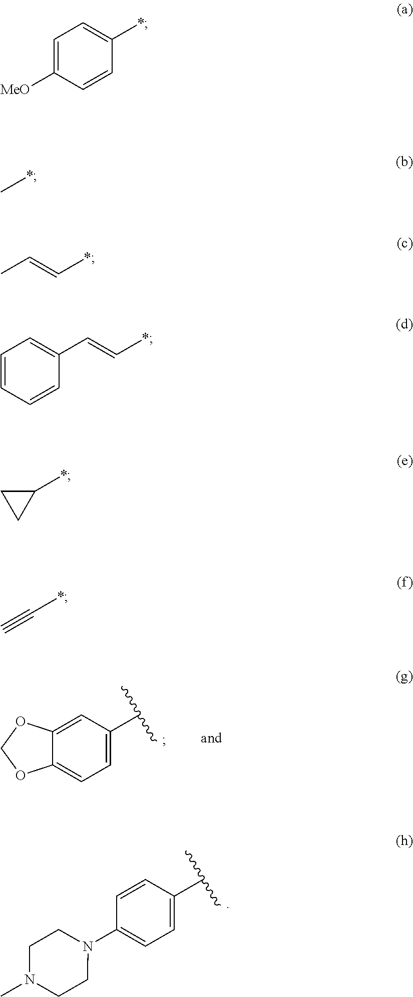

Preferred conjugates of the first aspect of the present disclosure may have a D^{L} of formula Ie:

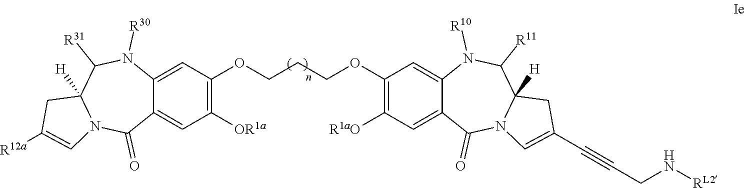

where R^{L2′}, R¹⁰, R¹¹, R³⁰ and R³¹ are as defined above

n is 1 or 3;

R^{1a} is methyl or phenyl;

R^{12a} is selected from:

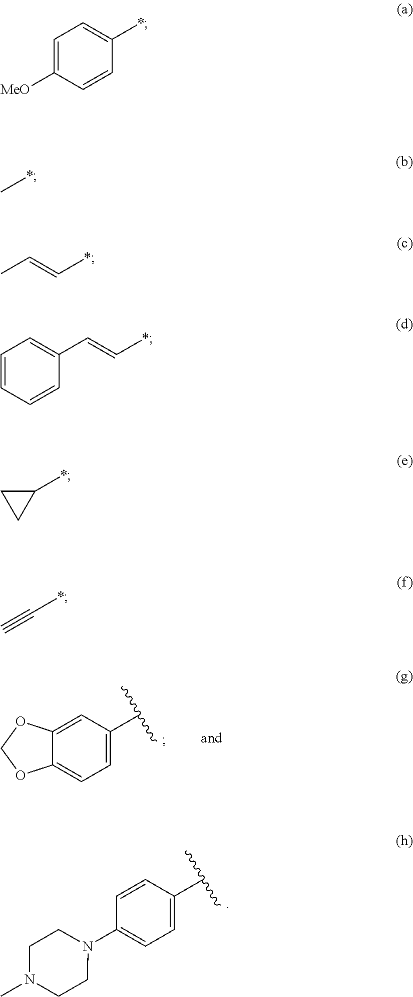

### BRIEF DESCRIPTION OF THE FIGURES

FIG. 1: AXL binding ELISA of fully humanised constructs. (2A) to Human AXL. (2B) to Cynomolgus monkey AXL.

FIG. 2: Accelerated stability analysis

### MATERIALS AND METHODS

Protocol 1: Production of DNA Plasmids for Expression

Materials
For heavy chain construct selection, Zeocin 25 μg/ml (Invivogen) was used.
For light chain construct selection, Blasticidin 100 μg/ml (Life Technologies) was used.

### Method

Transformed bacteria were spread on LB-agar Zeocin/Blasticidin plates, as required, incubated overnight at 37 C, then colonies were picked from each plate.

VH or VK colonies/glycerol stocks were inoculated into 3 ml LB containing Zeocin 25 μg/ml or Blasticidin 50 μg/ml, respectively.

p21, p27, pAdvantage (Promega) and pSVLT (generous gift from Tom Vink) were inoculated in LB-Ampicillin and shaken overnight.

3 ml overnight colony was seeded into 200 ml of LB-antibiotic and shaken overnight. DNA plasmids were isolated from each culture using the Promega PureYield™ Plasmid Maxiprep Kit following the manufacturer's instructions.

Protocol 2. Transient Transfection of HEK293T Cells with Expression Constructs

### Materials

Cells: HEK293T cells
Culture medium: DMEM high glucose 4.5 g/L (PAA) with 10% v/v FCS, penicillin and streptomycin
Fugene HD transfection reagent (Promega # E2311)
Opti-MEM (Life Technologies #11058-021) or
FreeStyle 293 Expression Medium (Life Technologies #12338-018)

### Method

Grow HEK293T cells in a T75 or T175 flask in a CO₂-gassed cell culture incubator. Split cultures 1:3 every 2 days or 1:4 to 1:5 every 3-4 days. The cells adhere weakly to the flasks and only a light trypsinisation is necessary to detach cells during passaging.

The day before transfection:
1. Trypsinise the cells, wash 1x in DMEM/10% FCS and count the cells.
2. Seed cells in a 6 well plate in 2 ml per well containing 2×10⁵ cells.

Next day, check cells are at least 80% confluent and replace the medium (2 ml/well).
1. 1.2 μg of total DNA (0.6 ug of each high and light chain DNA) is needed for each transfection and better results are obtained if the DNA concentration is at or above 90 ng/μl.
2. Add 0.6 ug of VH and 0.6 ug VK expression plasmid DNAs into of Fugene HD (4.5 μl) and OptiMEM/Freestyle medium, in a total volume of 60 ul, avoiding touching the sides of the tube with the Fugene HD.
3. Mix and leave at RT for 15 minutes.
4. Add Fugene mixture drop-wise around the well of HEK293T cells.
5. Return the 6-well plate to the CO₂-gassed cell culture incubator for 4 days.
6. Harvest each conditioned medium, centrifuge, and store at 4° C.

### Protocol 3: IgG Quantitation by ELISA

### Materials

Nunc-Immuno Plate MaxiSorp (Life Technologies, 43945A)
Goat Anti-Human IgG(Fc)-AffiniPure: Stratech Scientific, 109-005-098-JIR; 1 mg: 1.3 mg/ml
Human IgG1/kappa antibody (Sigma, 1-3889-1 mg: 1 mg/ml)
Goat anti-human kappa light chain peroxidase conjugate (Sigma, A-7164-1 ml)
1-Step Turbo TMB-ELISA, 250 mL (Thermo Scientific: #34022)
Acid stop=0.1M HCL
Sample enzyme conjugate (SEC) buffer Tween 20 (0.02% v/v), BSA 0.2% (w/v) in PBS
Washing buffer: 1×PBS, Tween 20 (0.1% v/v)

### Method

1. Coat each well of a 96-well immunoplate with 100 μl aliquots of 0.4 μg/ml (dilute stock×3000=10 ul in 30 ml: coat 5 ml per plate) goat anti-human IgG antibody, diluted in PBS, incubate overnight at 4° C. (or 37 C 1 hr). (Plates may be stored for 1 month at this stage). Also coat another blank plate with BSA/PBS blocking solution.
2. wash plate 3x with 200 μl/well of washing buffer.
3. Block coated plate: add 200 ul 3% BSA in PBS: incubate 37 C 1 hr
4. Into blank plate, dispense 200 μl SEC buffer into all wells except row B, cols 2-11 (blue, below).
5. Prepare 1 ug/ml solution of the human IgG1/kappa antibody in SEC buffer (×1000 diln)

Blocked Uncoated Plate:
6. Pipette 50 μl/well of stds/unknowns into rows A-H, cols 1 and 7 (makes a ×5 dilution).
7. Serially transfer 100 μl across plate to achieve serial ×3 dilution series.
8. Transfer 100 μl from each well to the corresponding well of the BLOCKED anti-IgG-COATED plate.

Blocked Anti-IgG Coated Plate:
9. Incubate at 37° C. for 1 hr. Rinse all the wells 3x with washing buffer (200 μl).
10. Dilute the goat anti-human kappa light chain peroxidase conjugate 5000-fold in SEC buffer and add 100 □l to each well. Repeat the incubation and washing steps (step 9).
11. Add 100 μl of TMB Turbo substrate to each well, incubate in the dark at room temperature for 10 min.
12. Stop the reaction by adding 50 μl of acid (0.1M HCl) to each well.
13. Read the optical density at 450 nm.

### Protocol 4: AXL Binding ELISA

### Materials

Human AXL-Strep-His was produced by Evitria AG in transiently transfected CHO cells and purified on Ni Sepharose High Performance (GE Healthcare 17-5268-01) following manufacturers instructions and stored in aliquots at −20 C.
Goat anti-human kappa light chain peroxidase conjugate (Sigma, A-7164-1 ml)
Nunc-Immuno Plate MaxiSorp (Life Technologies, 43945A)
Plate washer: Biotek LS405
3% BSA: BSA 3% w/v in PBS
PBS Tween: Tween 20 0.05% v/v in PBS
PBS/Tween/BSA: BSA 0.5% w/v in PBS/Tween
1-Step Turbo TMB-ELISA (Thermo Scientific #3402)

### Protocol

1. Dispense 50 μl/well of human AXL-strep-His (1 ug/ml in PBS)
2. Cover with adhesive plate sealer and incubate at 4 C overnight.
3. Block: Dispense 50 μl/well of 3% BSA and incubate for 1 hr 37 C,
4. Wash plate with PBS/Tween 3x
5. Serially 3-fold dilute 5E5 antibodies (2 ml HEK293T culture supernatants) on non-binding polypropylene plate in PBS/Tween/BSA: serially transfer 50 ul onto 100 ul.
6. Transfer 50 ul from antibody dilution plate onto washed, blocked AXL-coated plate
7. Incubate 37 C 1 hr
8. Wash plate with PBS/Tween 3×
9. Dispense anti-human IgG-HRP conjugate, diluted 1:1000 in PBS/Tween/BSA
10. Incubate 37 C 1 hr
11. Wash plate with PBS/Tween 3
12. Wash plate with PBS 3×
13. Dispense 100 ul/well 1-Step Turbo TMB-ELISA substrate solution
14. Incubate 30 min at room temperature (or less if reaction is rapid)
15. Dispense 100 ul/well 0.6M HCl to stop the substrate reaction
16. Measure optical density at 450 nm

### Protocol 4A: SPR Measurement of Antibody Affinity

### Materials

1. Sensor Chip CM5 Biacore; Cat. # BR-1000-14
2. Amine Coupling Kit (EDC, NHS, ethanolamine-HCl) Biacore; Cat. # BR-1000-50
3. Immobilization buffer (10 mM Na acetate, pH 4.0) Biacore; Cat. # BR-1003-49
4. 50 mM NaOH Biacore; Cat. # BR-1003-58
5. Running buffer (PBS/Tween20 0.05% v/v)
6. Biacore T200 GE Healthcare
7. Regeneration solution: 10 mM HCl, 1 M NaCl

### Coupling Method

Activate flow cell 2 with NHS-EDC 420s at 5 μl/min, then inject human AxI-Strep-His (Evitria) (10 μg/mL in 10 mM sodium acetate, pH 4.0) to achieve a coupling of 10⁻²⁰ RU. Block with ethanolamine for 420s. Repeat the process for flow cell 1 but with no antigen to create a reference flow cell. 12RU AXL fusion protein was coated.
For the Fc fusion, Human Axl-Fc chimera (R&D Systems #154-AL) (5 μg/mL in 10 mM sodium acetate, pH 4.5) was used with the above protocol. 16RU of AXL-Fc was coated.
   Protocol with AXL-Strep-His Antigen
Serial 10x dilutions of antibody, from 3000 nM, were made in PBS/Tween20. 2x regeneration cycles were used with 10 mM HCl, 1 M NaCl for 30s at 30 μl/min for both cycles. Start-up solution was PBS/Tween20 and set to 3 cycles.
Sample injection parameters were 120s at 30 μl/min, with 600s dissociation time.
Prime and normalise detector were run before sample application with experimental conditions 25 C and sample storage at 4 C.
Kinetic analysis used BIAevaluation software with bivalent ligand binding model. For chimeric 1H12 with AXL-Strep-His, “heterogeneous ligand” binding model was used due to poor fit with bivalent or monovalent algorithms.
Each antibody dilution was injected twice.
   Protocol with AXL-Fc Chimera Antigen
The above protocol was used except that the running buffer was HBSEP+, serial 10x dilutions of antibody were from 500 nM in HBSEP+ running buffer and were injected for 180 sec.
Analysis used BIAevaluation software with the bivalent binding model.

### Protocol 5: Capillary Isoelectric Focussing

### Materials

PA 800 plus (AB SCIEX)
Anolyte Solution: 200 mM Phosphoric Acid (Sigma-Aldrich #345 245).
4.3M Urea (Sigma-Aldrich #U0631) in water.
Catholyte Solution: 300 mM Sodium Hydroxide.
3M Urea (Sigma-Aldrich #U0631) in cIEF Gel (Beckman Coulter #477497).
Chemical Mobiliser: 350 mM Acetic Acid (Sigma-Aldrich #537 020).
Pharmalyte 3-10 (GE Healthcare 17-0456-01).
Cathodic Stabiliser: 500 mM Arginine (Sigma-Aldrich #A5006).
cIEF Peptide markers PI 4-10 (Beckman Coulter # A58481).
Anodic Stabiliser: 200 mM Iminodiacetic Acid (Sigma-Aldrich #220 000).
Neutral Capillary, 50 μm i.d.×45 cm, (Beckman Coulter #477 441)

### Method

Turn on the PA800+ machine and UV lamp, allowing it to warm up 30 mins before use.
Clean the system electrodes and interface block with a damp Kimwipe.
Prepare buffer trays as shown, with 1.5 mL reagent per vial, 1 mL of water in the waste and place in the system.
1. DDI water
2. Anolyte
3. Urea Solution
4. 3M urea/cIEF Gel
5. Chemical Mobilizer
6. Waste
7. Catholyte
8. Chemical Mobilizer
9. BI (Inlet Buffer Tray)
10. BO (Outlet Buffer Tray)
NOTE. Each set of buffer vials is good for 6 consecutive runs or for 24 hours inside the instrument.
Insert the capillary cartridge into the system and close the front panel.
NOTE. Do not expose the neutral-coated capillary ends to air for more than fifteen min. When the capillary is not in use, submerge the capillary ends in vials filled with DDI water.
Prepare cIEF master mix using the table below. Dispense each reagent into a centrifuge tube, vortex 1 min, invert every 15-20 sec to ensure complete mixing and store at 2° C. to 8° C. for up to 1 day.

| Reagent | Volume per sample (μl) |
|---|---|
| 3M Urea-cIEF Gel | 100 |
| Pharmalyte 3-10 | 12 |
| Cathodic Stabiliser | 20 |
| Anodic Stabiliser | 2 |
| pI marker 10 | 2 |
| pI marker 9.5 | 2 |
| pI marker 7 | 2 |
| pI marker 5.5 | 2 |
| pI marker 4.1 | 2 |

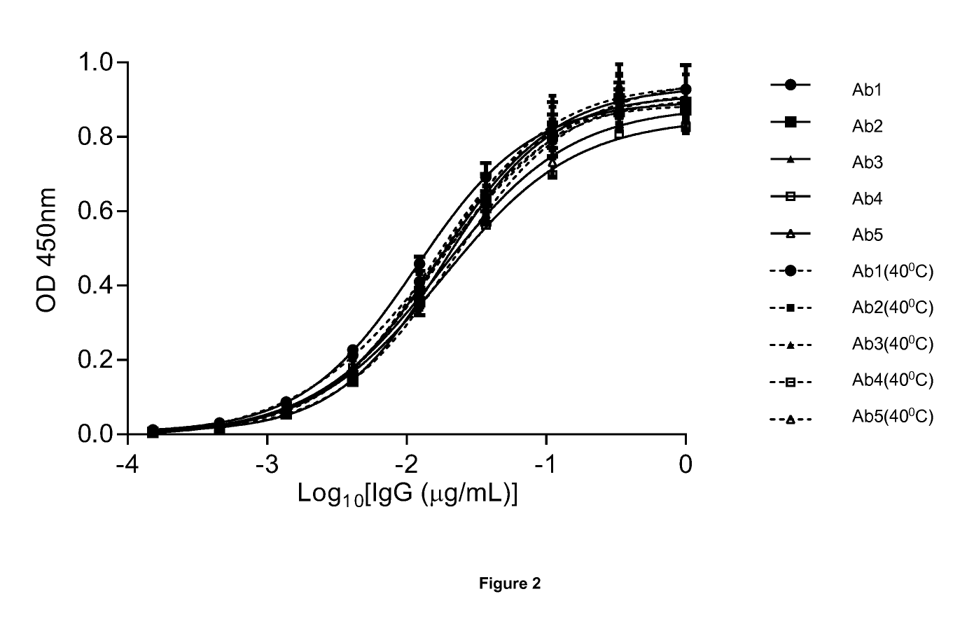

Desalt and concentrate each antibody (to about 5 mg/ml) in 2M urea using Amicon Ultra 0.5 mL centrifugal filters (Sigma Z677108).
Mix 200 μL of master mix with 10 μL of protein, vortex the cIEF sample for 1 min, inverting the tube every 15-20 sec, then centrifuge at high speed to remove any air bubbles. Transfer 100 μL of sample into a micro vial. Place the micro vial into a universal plastic vial and cap it with a blue cap. Then place the sample vial in the inlet sample tray.
Run the “cIEF Conditioning—PA 800 plus.met” method to condition the column.
   Rinse for 5 min at 50 psi with Chemical Mobilizer. See FIG. 2.10.
   2 Rinse for 2 min at 50 psi with DDI water.
   3 Rinse for 5 min at 50 psi with cIEF gel.
   4 Submerge both of the capillary ends in vials filled with DDI water.
Use the “cIEF Separation—PA 800 plus.met” method to create a sequence containing all protein samples and a blank.
   Rinse for 3 min at 50 psi with Urea Solution. See FIG. 2.11.
   Rinse for 2 min at 50 psi with DDI water.
   Inject sample for 99.0 sec at 25 psi.
   Water dip by submerging both capillary ends in DDI water.
   Focusing step, 15 min at 25 kV under normal polarity (Time=0).
   Chemical mobilization, 30 min at 30 kV under normal polarity (Time=15 min).
   Stop data collection (Time=45 min).
   Rinse for 2 min at 50 psi with DDI water (Time=45.10 min).
   Submerged both of the capillary ends in DDI water (Time=47.20 min).
   End the method (Time=47.30 min).
Put the “cIEF Shutdown—PA 800 plus.met” method at the end of the sequence to rinse the capillary and turn off the UV.
   Rinse for 2 min at 50 psi with DDI water. See FIG. 2.12.
   Rinse for 5 min at 50 psi with cIEF gel.
   Turn off the UV lamp.
   Submerge both of the capillary ends in vials filled with DDI water.
For short term storage (1 to 3 days), leave the capillary on the instrument. For long term storage (over 3 days), place the capillary cartridge in the storage box with both ends submerged in water tubes and store upright at 4 C.

### Protocol 6: Protein Thermal Shift Protocol

### Materials

96 well optical plate semi-skirted (Starlab cat.L1402-9700).
Protein thermal shift dye kit (Life Technologies cat.446148).
Microamp optical adhesive film (Applied Biosystems)
7500 Real-Time PCR System (Life Technologies)
Test items: Antibodies from Evitria and Spirogen
Protein thermal shift v1.2 software (Life Technologies)

### Method

Protein thermal shift dye (2.5 μL 1:1000 dilution) was added to sample proteins (17.5 μL of 0.5 mg/mL in PBS) in a 96 well optical plate and mixed thoroughly. Every sample was done in quadruplicate. The plate was sealed with a optical adhesive film and bubbles in the wells were removed by centrifugation 1 min at 500 g, then placed on ice. The sealed plate was introduced in the 7500 Real-Time PCR System and subsequently the experiment was set up as follows:

| | |
|---|---|
| Experiment Name | Name (using up to 100 letters/numbers) |
| Instrument type | 7500 Fast (96 Wells) or 7500 (96 Wells) |
| Experiment type | Melt Curve |
| Reagent type | Other |
| Ramp speed | Standard |
| Reporter | ROX |
| Quencher | None |
| Passive Reference | None |

Temperature Cycling on the ABI 7500 Set Up:

Reaction vol 20 μl
Ramp mode continuous

| Step | Ramp rate | Temp ° C. | Time (mm:ss) |
|---|---|---|---|
| 1 | 100% | 25.0 | 02:00 |
| 2 | 1% | 99.0 | 02:00 |

Data analysis and derivation of Tm data were done using the software following the manufacturers instructions.

### Protocol 7: HPLC Size Exclusion Chromatography

### Materials

Shimadzu HPLC system, or equivalent, consisting of the following, or equivalent:
2x DGU-20A_{5R} Prominence Degassing units
2x LC-20AD_{XR} Nexera Pumps
SIL-20AC_{XR} Nexera Autosampler
CTO-20AC Prominence Column oven
SPD-M30A Prominence DAD detector
Computer with LabSolutions software.
TSKgel Super SW mAb HTP 4 um 4.6 mm×15 cm
200 mM Potassium Phosphate, 250 mM Potassium Chloride, 10% v/v i-Propanol, pH 6.95.

### Sample Preparation

Mobile Phase: 200 mM Potassium Phosphate, 250 mM Potassium Chloride, 10% v/v i-Propanol, pH 6.95.
Analytical Sample: Inject 2-20 μL of neat ADC sample (at least 1 mg/ml for best results). Typically 10 μL of 5 mg/ml gives good results.

HPLC Parameters
Method File name: MSOP-018
HPLC Column: TSKgel Super SW mAb HTP 4 um 4.6 mm×15 cm
Flow Rate: 0.5 ml/min
Injection volume: 2-20 μL
Detection, UV: 280 nm
   330 nm (for information only)
Column Temp: ambient temperature
Autosampler Temp: 15° C.
Gradient: Isocratic

### Method

Intact ADCs typically elute at ˜16-18 minutes.

Aggregates typically elute at ˜11-14 minutes.

Low molecular weight species typically elute at ˜>20 minutes.

### Protocol 8: Hydrophobic Interaction Chromatography

### Materials

HPLC system, or equivalent, consisting of the following, or equivalent:
SRD-3600 SOLVENT RACK, 6 DEGASS. LINES
HPG-3400RS PUMP (Thermo Scientific)
HPG-3200RS PUMP(Thermo Scientific)
WPS-3000TFC ANALYTICAL AUTOSAMPLER (Thermo Scientific)
TCC-3000RS COLUMN THERMOSTAT (Thermo Scientific)
DAD-3000RS DETECTOR (Thermo Scientific)
Computer with Chromeleon software (Thermo Scientific)
Proteomix HIC Butyl-NP5, 5 um, non-porous, 4.6×35 mm (Sepax) column
Ammonium sulfate ((NH₄)₂SO₄)
Sodium acetate (NaOAc)
i-Propanol
Water, HPLC grade
Mobile Phase A: 1.25 M (NH₄)₂SO₄, 25 mM NaOAc (pH 5.50)
Mobile Phase B: 75% 25 mM NaOAc (pH 5.50), 25% i-Propanol
Blank Solution: Water

### Sample Preparation

Analytical Sample: <10 μL of neat ADC sample at a concentration of 1-5 mg/mL.

### HPLC Parameters

Method File name: HIC Gradient 1 AB
HPLC Column: Proteomix HIC Butyl-NP5, 5 um, non-porous, 4.6×35 mm (Sepax)
Flow Rate: 0.8 ml/min
Injection volume: <10 μL
Detection, UV: 214 nm
   330 nm (for information only)
Column Temp: 25° C.
Autosampler Temp: 10° C.

### Gradient

| Time (min) | % B |
|---|---|
| 0 | 0 |
| 1 | 0 |
| 13 | 100 |
| 14 | 100 |
| 14.1 | 0 |
| 18 | 0 |

### Method

Flush the flow-path of the HPLC and column with water. Set up the HPLC under the operating conditions outlined above and equilibrate the system for a minimum of 10 minutes.

### Protocol 9: Reverse Phase Chromatography

### Materials

HPLC system, or equivalent, consisting of the following, or equivalent:
SRD-3600 SOLVENT RACK, 6 DEGASS. LINES
HPG-3400RS PUMP (Thermo Scientific)
HPG-3200RS PUMP(Thermo Scientific)
WPS-3000TFC ANALYTICAL AUTOSAMPLER (Thermo Scientific)
TCC-3000RS COLUMN THERMOSTAT (Thermo Scientific)
DAD-3000RS DETECTOR (Thermo Scientific)
Computer with Chromeleon software (Thermo Scientific)
Aeris Widepore XB-C18, 200 Å, 3.6 μm, 2.1×150 mm (Phenomenex, 00F-4482-AN)
Acetonitrile, HPLC grade.
Water, HPLC grade.
Trifluoroacetic acid (TFA), HPLC grade.
100 mM NaBorate, pH 8.4
500 mM DTT
49:49:2 Acetonitrile/Water/Formic acid

Mobile Phase A: Water+0.1% v/v TFA.

Mobile Phase B: Acetonitrile+0.1% v/v TFA.

Blank Solution: 1:1 v/v Acetonitrile/Water.

### Sample Preparation

To 40 μL of sample (5 mg/ml) add water, 30 μl, NaBorate, 20 μl, and DTT (500 mM), 10 μL. Incubate at 37° C., 30 min, then add 100 μl of 49:49:2 Acetonitrile/Water/Formic acid.

### HPLC Parameters

Method File name: RP_Aeris_Column6
HPLC Column: Aeris Widepore XB-C18, 200A, 3.6 μm, 2.1×150 mm (Phenomenex, OOF-4482-AN)
Flow Rate: 1.0 ml/min
Injection volume: 10 μl (or full loop)
Detection, UV: 214 nm
   330 nm (for information only)
Column Temp: 80° C.
Autosampler Temp: 15° C.

Gradient

| Time (minutes) | % B |
|---|---|
| 0 | 22.5 |
| 1 | 22.5 |
| 11 | 50 |
| 11.5 | 90 |
| 13.5 | 90 |
| 14.5 | 22.5 |
| 16 | 22.5 |

### Method

Set up the HPLC under the operating conditions outlined above and equilibrate the system for a minimum of 10 minutes. Inject a blank sample, followed by the sample for analysis.

### EXAMPLES

### General Experimental Methods

Optical rotations were measured on an ADP 220 polarimeter (Bellingham Stanley Ltd.) and concentrations (c) are given in g/100 mL. Melting points were measured using a digital melting point apparatus (Electrothermal). IR spectra were recorded on a Perkin-Elmer Spectrum 1000 FT IR Spectrometer. ¹H and ¹³C NMR spectra were acquired at 300 K using a Bruker Avance NMR spectrometer at 400 and 100 MHz, respectively. Chemical shifts are reported relative to TMS (6=0.0 ppm), and signals are designated as s (singlet), d (doublet), t (triplet), dt (double triplet), dd (doublet of doublets), ddd (double doublet of doublets) or m (multiplet), with coupling constants given in Hertz (Hz). Mass spectroscopy (MS) data were collected using a Waters Micromass ZQ instrument coupled to a Waters 2695 HPLC with a Waters 2996 PDA. Waters Micromass ZQ parameters used were: Capillary (kV), 3.38; Cone (V), 35; Extractor (V), 3.0; Source temperature (° C.), 100; Desolvation Temperature (° C.), 200; Cone flow rate (L/h), 50; De-solvation flow rate (L/h), 250. High-resolution mass spectroscopy (HRMS) data were recorded on a Waters Micromass QTOF Global in positive W-mode using metal-coated borosilicate glass tips to introduce the samples into the instrument. Thin Layer Chromatography (TLC) was performed on silica gel aluminium plates (Merck 60, F₂₅₄), and flash chromatography utilised silica gel (Merck 60, 230-400 mesh ASTM). Except for the HOBt (NovaBiochem) and solid-supported reagents (Argonaut), all other chemicals and solvents were purchased from Sigma-Aldrich and were used as supplied without further purification. Anhydrous solvents were prepared by distillation under a dry nitrogen atmosphere in the presence of an appropriate drying agent, and were stored over 4A molecular sieves or sodium wire. Petroleum ether refers to the fraction boiling at 40-60° C.

### General LC/MS Conditions:

The HPLC (Waters Alliance 2695) was run using a mobile phase of water (A) (formic acid 0.1%) and acetonitrile (B) (formic acid 0.1%). Gradient: initial composition 5% B held over 1.0 min, then increase from 5% B to 95% B over a 3 min period. The composition was held for 0.1 min at 95% B, then returned to 5% B in 0.03 minutes and hold there for 0.87 min. Total gradient run time equals 5 minutes.

Flow rate 3.0 mL/min, 400 μL was split via a zero dead volume tee piece which passes into the mass spectrometer. Wavelength detection range: 220 to 400 nm. Function type: diode array (535 scans). Column: Phenomenex Onyx Monolithic C18 50×4.60 mm.

The reverse phase flash purification conditions were as follows: The Flash purification system (Varian 971-Fp) was run using a mobile phase of water (A) and acetonitrile (B). Gradient: initial composition 5% B over 20 C.V. (Column Volume) then 5% B to 70% B within 60 C.V. The composition was held for 15 C.V. at 95% B, and then returned to 5% B in 5 C.V. and held at 5% B for 10 C.V. Total gradient run time equals 120 C.V. Flow rate 6.0 mL/min. Wavelength detection range: 254 nm. Column: Agilent AX1372-1 SF10-5.5gC8.

Preparative HPLC: Reverse-phase ultra-high-performance liquid chromatography (UPLC) was carried out on Phenomenex Gemini NX 5μ C-18 columns of the following dimensions: 150×4.6 mm for analysis, and 150×21.20 mm for preparative work. All UPLC experiments were performed with gradient conditions. Eluents used were solvent A (H₂O with 0.1% Formic acid) and solvent B (CH₃CN with 0.1% Formic acid). Flow rates used were 1.0 ml/min for analytical, and 20.0 ml/min for preparative HPLC. Detection was at 254 and 280 nm.

### Example 1: Characterization of Humanized Antibodies

The five antibodies described below were produced, expressed, and quantified according to Protocols 1-3 described herein. The expression levels recorded are shown below in Table 1.
Ab1 is an anti-AXL antibody comprising a VH domain having the sequence according to SEQ ID NO. 1, a VL domain having the sequence according to SEQ ID NO. 4, and a constant region derived from one or more human antibodies.
Ab2 is an anti-AXL antibody comprising a VH domain having the sequence according to SEQ ID NO. 2, a VL domain having the sequence according to SEQ ID NO. 5, and a constant region derived from one or more human antibodies.
Ab3 is an anti-AXL antibody comprising a VH domain having the sequence according to SEQ ID NO. 2, a VL domain having the sequence according to SEQ ID NO. 7, and a constant region derived from one or more human antibodies.
Ab4 is an anti-AXL antibody comprising a VH domain having the sequence according to SEQ ID NO. 3, a VL domain having the sequence according to SEQ ID NO. 5, and a constant region derived from one or more human antibodies.
Ab5 is an anti-AXL antibody comprising a VH domain having the sequence according to SEQ ID NO. 3, a VL domain having the sequence according to SEQ ID NO. 7, and a constant region derived from one or more human antibodies.

A stability assay was performed during which the antibodies were heated in sterile PBS at 40° C. for 60 hr and analysed for aggregation by SEC and for binding activity by human AXL ELISA. No increase in aggregation or loss in AXL binding activity was detected for any of the antibodies (see FIG. 2).

The antibodies were further characterised using Protocols 5-9 as described herein. The results are shown below in Table 1 (all assays were performed on the HEK293F expression product unless otherwise stated; “F”=HEK293F, “T”=HEK293T).

TABLE 1

| | | | | | | | | | | RP HPLC | |
|---|---|---|---|---|---|---|---|---|---|---|---|
| | Expression | | | SEC % | | HIC | | | | LC2 peak | |
| | (μg/mL) | | | monomer | | retention | | | | % protein | |
| | in . . . | | | 280 nm | | time min | | | Tm | 280 nm | |
| Antibody | T | F | CHO | CHO | F | F | CHO | pl | (° C.) | F | CHO |
| Ab1 | 33.7, | 7.16 | 366 | 97.9 | 87% | 4.5 | 4.5 | 8.05 | 69.52 | 0 | 0 |
| | 40.1 | | | | | | | | | | |
| Ab2 | 18.2 | 2.55 | 265 | 97.6 | 86% | 5.2 | 5.2 | 8.06 | 59.71 | 6.4% | 5.9% |
| Ab3 | 15.2 | 2.24 | 287 | 98.3 | 88% | 4.8 | 4.7 | 8.15 | 62.05 | 0 | 0 |
| Ab4 | 17.8 | 1.91 | 306 | 96.9 | 96% | 5.6 | 5.6 | 7.56 | 60.32 | 8.1% | 5.9% |
| Ab5 | 16.5 | 1.95 | 298 | 97.7 | 98% | 5.0 | 4.9 | 7.54 | 63.06 | 0 | 0 |

Binding of the antibodies to AXL antigens indicated that binding was unusually sensitive to both antigen preparation and presentation and antibody geometry.

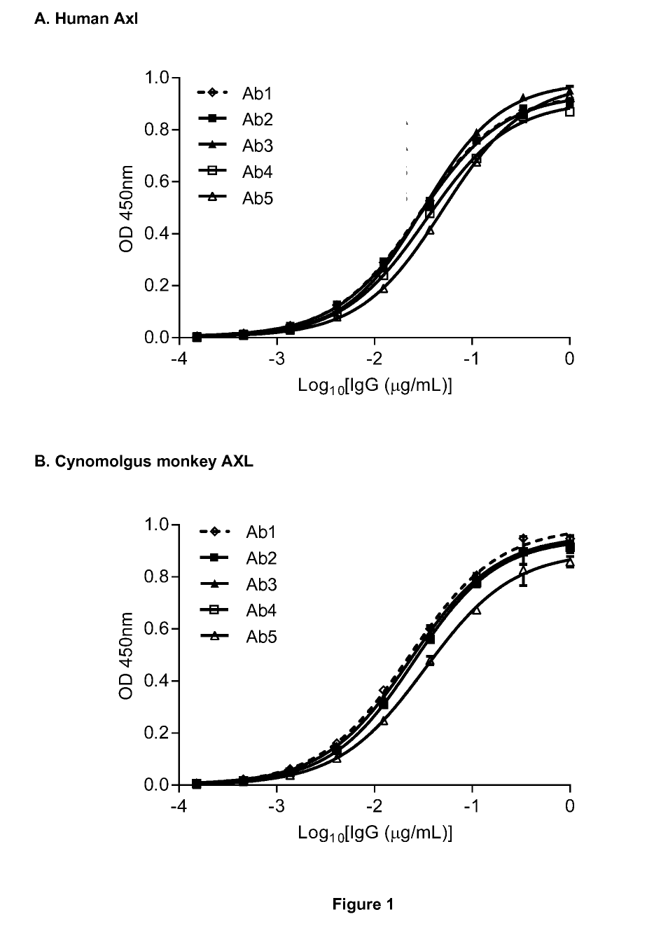

Initial measurements by ELISA using Axl-Strep-His antigen, as disclosed in Protocol 4 suggested that the binding of antibodies comprising humanised 1H12 heavy and light chains (Ab2—Ab5) were broadly similar to the antibody comprising the murine VH and VL domains (Abl) (see FIG. 1).

SPR measurements of antibody affinity using Axl-Strep-His antigen indicated that Ab2, Ab3, and Ab5 had higher affinity for Axl-Strep-His than Ab1 (see Table 2).

SPR measurements of antibody affinity using Axl-Fc antigen indicated that Ab2 and Ab4 had higher affinity for Axl-Fc than Ab1 (see Table 2).

TABLE 2

| | AXL-Strep-His antigen | | | AXL-Fc antigen | | |
|---|---|---|---|---|---|---|
| | ka | | KD1 | | | KD |
| Antibody | (1/Ms) | kd (1/s) | (nM) | ka (1/Ms) | kd (1/s) | (nM) |
| Ab1 | 1.31E+04 | 5.33E−04 | 40.7** | 3.09E+04 | 1.42E−04 | 4.6 |
| Ab2 | 1.32E+04 | 4.41E−04 | 33.4 | 8.37E+04 | 1.54E−04 | 1.84 |
| Ab3 | 1.92E+04 | 6.36E−04 | 33.1 | 2.45E+04 | 2.00E−04 | 8.5 |
| Ab4 | 9759 | 5.90E−04 | 60.4 | 2.90E+05 | 3.96E−04 | 1.37 |
| Ab5 | 1.89E+04 | 4.35E−04 | 23 | 2.06E+04 | 1.59E−04 | 7.69 |

**The observed binding data for 1H12 chimeric antibody did not fit monovalent or bivalent algorithms well, so in this one case, a “heterogeneous ligand” model was used. For all other binding data, the bivalent ligand model was used.

### Example 2: Cytotoxicity of Anti-AXL ADCs in MDA-MB-231 Cells

### Method

Adherent MDA-MB-231 cells (80-90% confluent) were washed with PBS, dissociated with trypsin-EDTA (5 ml), diluted with F12K culture medium (10 ml), containing FCS (10%), centrifuged (400 g for 5 min) and re-suspended in culture medium (10 ml). Cells were counted (Trypan blue), diluted to 2×105/ml, dispensed in 96-well flat bottom micro-plates (50 μl/well) and incubated overnight to allow re-adherence.
A stock solution (1 ml) of antibody drug conjugate (ADC) (20 μg/ml) was made by dilution of filter-sterile ADC into cell culture medium. Eight 10-fold dilutions of ADC were made by serial transfer of 100 μl into 900 μl of culture medium. Each ADC dilution was dispensed (50 μl/well) into four wells containing MDA-MB-231 cells (50 μl), seeded the previous day. Control wells received cell culture medium (50 μl).
The micro-plate, containing cells and ADCs, was incubated at 37° C. in a 5% CO2-gassed incubator for 5 days. After incubation, cell viability was measured by MTS assay. MTS (CellTiter 96® AQueous One Solution Cell Proliferation Assay: Promega) was dispensed (20 μl per well) into each well and the plate returned to the incubator for 4 hr.
Absorbance of each well was measured at 490 nm. Percent cell survival was calculated from the mean absorbance in the four ADC-treated wells compared to the mean absorbance in the four control wells (100%).

### Results and Discussion

The results of the cytoxicity experiments are shown in Table 3.

TABLE 3

| ADC | DAR | GI₅₀ (pg/ml) |
|---|---|---|
| Ab1-ConjE | 2.2 | 6.953 |
| Ab2-ConjE | 2.4 | 73.01 |
| Ab3-ConjE | 2.2 | 27.61 |
| Ab4-ConjE | 2.6 | 64.2 |
| Ab5-ConjE | 2.3 | 44.03 |
| B12-ConjE | 2.7 | 1776 |

Despite the similar AXL-Strep-His binding ELISA EC50 of antibodies comprising humanised 1H12 heavy and light chains (Ab2-Ab5) and the murine VH and VL domains (Abl) humanised and chimeric 1H12 antibodies, the MDA-MB-231 cytotoxicity of the same antibodies conjugated to the ConjE payload (Table 3) showed a greater cytoxicity of Ab1 (GI₅₀ 6.9 μg/ml) versus Ab2-Ab5 (GI₅₀ 27-73 μg/ml). This may indicate that the presentation of AXL 1H12 epitope on cells is subtly different from that in recombinant fusion protein attached to a plastic surface.

### Example 2—Alternative Synthesis of Compound 83

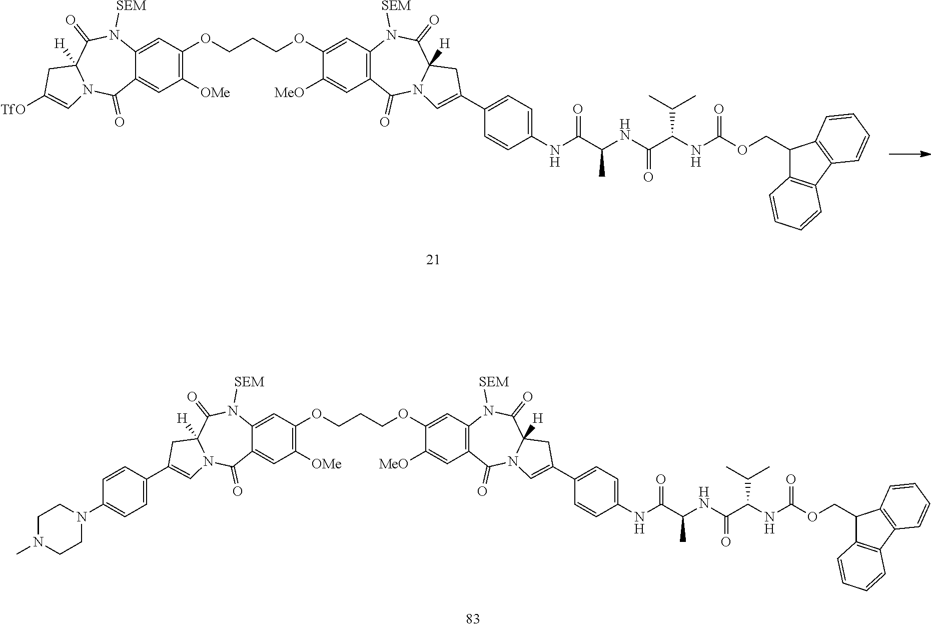 [SMILES: COc1cc2c(cc1OCCCOc1cc3c(cc1OC)C(=O)N1C=C(c4ccc(NC(=O)[C@H](C)NC(=O)[C@@H](NC(=O)OCC5c6ccccc6-c6ccccc65)C(C)C)cc4)C[C@H]1C(=O)N3COCC[Si](C)(C)C)N(COCC[Si](C)(C)C)C(=O)[C@@H]1CC(OS(=O)(=O)C(F)(F)F)=CN1C2=O ; COc1cc2c(cc1OCCCOc1cc3c(cc1OC)C(=O)N1C=C(c4ccc(N5CCN(C)CC5)cc4)C[C@H]1C(=O)N3COCC[Si](C)(C)C)N(COCC[Si](C)(C)C)C(=O)[C@@H]1CC(c3ccc(NC(=O)[C@H](C)NC(=O)[C@@H](NC(=O)OCC4c5ccccc5-c5ccccc54)C(C)C)cc3)=CN1C2=O]

(9H-fluoren-9-yl)methyl ((S)-1-(((S)-1-((4-((S)-7-methoxy-8-(3-(((S)-7-methoxy-2-(4-(4-methylpiperazin-1-yl)phenyl)-5,11-dioxo-10-((2-(trimethylsilyl)ethoxy)methyl)-5,10,11,11a-tetrahydro-1H-pyrrolo[2, 1-c][1,4]benzodiazepin-8-yl)oxy)propoxy)-5,11-dioxo-10-((2-(trimethylsilyl)ethoxy)methyl)-5,10,11,11a-tetrahydro-1H-pyrrolo[2,1-c][1,4]benzodiazepin-2-yl)phenyl)amino)-1-oxopropan-2-yl)amino)-3-methyl-1-oxobutan-2-yl)carbamate (83) PBD-triflate 21 (469 mg, 0.323 mmol)(Compound 21 in WO 2014/057073), boronic pinacol ester (146.5 mg, 0.484 mmol) and Na₂CO₃ (157 mg, 1.48 mmol) were dissolved in a mixture of toluene/MeOH/H₂O, 2:1:1 (10 mL). The reaction flask was purged with argon three times before tetrakis(triphenylphosphine)palladium(0) (7.41 mg, 0.0064 mmol) was added and the reaction mixture heated to 30° C. overnight. The solvents were removed under reduced pressure and the residue was taken up in H₂O (50 mL) and extracted with EtOAc (3×50 mL). The combined organics were washed with brine (100 mL), dried with MgSO₄, filtered and the volatiles removed by rotary evaporation under reduced pressure. The crude product was purified by silica gel column chromatography (CHCl₃ 100% to CHCl₃/MeOH 95%:5%) to afford pure 83 in 33% yield (885 mg). LC/MS 3.27 min (ES+) m/z (relative intensity) 1478 ([M+H]⁺, 100%).

### Example 3

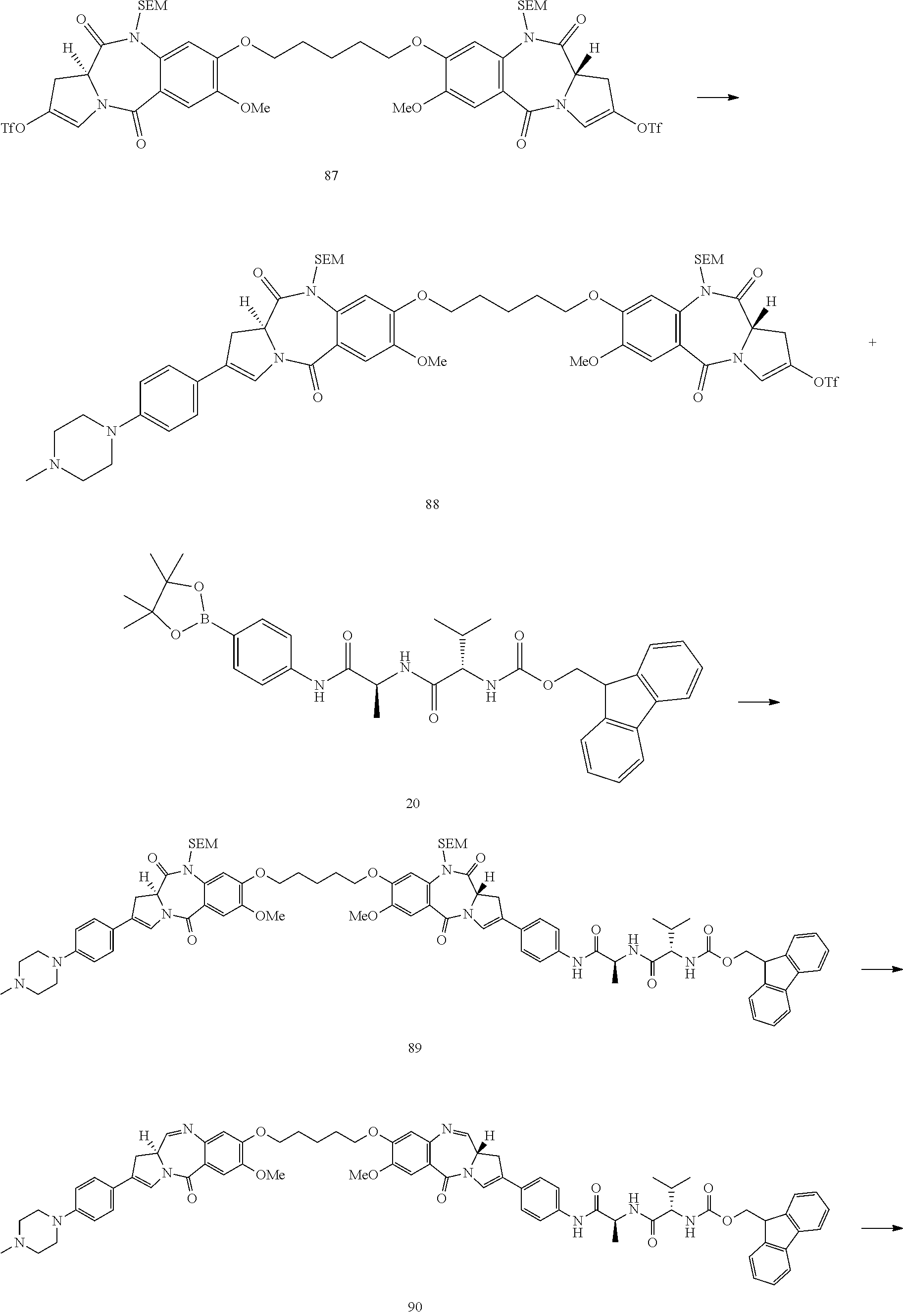

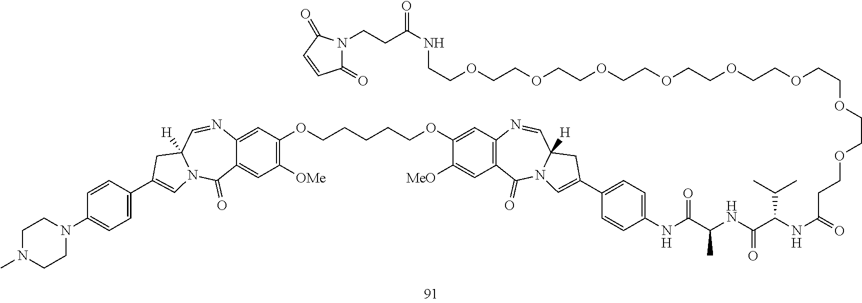 [SMILES: COc1cc2c(cc1OCCCCCOc1cc3c(cc1OC)C(=O)N1C=C(c4ccc(N5CCN(C)CC5)cc4)C[C@H]1C=N3)N=C[C@@H]1CC(c3ccc(NC(=O)[C@H](C)NC(=O)[C@@H](NC(=O)CCOCCOCCOCCOCCOCCOCCOCCOCCNC(=O)CCN4C(=O)C=CC4=O)C(C)C)cc3)=CN1C2=O]

### (a) (S)-7-methoxy-8-((5-(((S)-7-methoxy-2-(4-(4-methylpiperazin-1-yl)phenyl)-5,11-dioxo-10-((2-(trimethylsilyl)ethoxy)methyl)-5,10,11,11a-tetrahydro-1H-benzo[e]pyrrolo[1,2-a][1,4]diazepin-8-yl)oxy)pentyl)oxy)-5,11-dioxo-10-((2-(trimethylsilyl)ethoxy)methyl)-5,10,11,11a-tetrahydro-1H-benzo[e]pyrrolo[1,2-a][1,4]diazepin-2-yl trifluoromethanesulfonate (88)

Pd(PPh₃)₄ (30 mg, 26 μmol) was added to a stirred mixture of the bis-enol triflate 87 (1 g, 0.87 mmol)(Compound 8b in WO 2010/043880), 4-(4-methylpiperazin-1-yl)phenylboronic acid, pinacol ester (264 mg, 0.87 mmol), Na₂CO₃ (138 mg, 1.30 mmol), EtOH (5 mL), toluene (10 mL) and water (5 mL). The reaction mixture was allowed to stir under a nitrogen atmosphere overnight at room temperature after which time the complete consumption of starting material was observed by TLC (EtOAc) and LC/MS (1.52 min (ES+) m/z (relative intensity) 1171.40 ([M+H]⁺, 100)). The reaction mixture was diluted with EtOAc (400 mL) and washed with H₂O (2×300 mL), brine (200 mL), dried (MgSO₄), filtered and evaporated under reduced pressure to provide the crude product. Purification by flash chromatography (gradient elution: 100:0 v/v EtOAc/MeOH to 85:15 v/v EtOAc/MeOH) afforded the asymmetrical triflate 88 (285 mg, 28%). ¹H NMR (400 MHz, CDCl3) δ 7.39 (s, 1H), 7.37-7.29 (m, 4H), 7.23 (d, J=2.8 Hz, 2H), 7.14 (t, J=2.0 Hz, 1H), 6.89 (d, J=9.0 Hz, 2H), 5.54 (d, J=10.0 Hz, 2H), 4.71 (dd, J=10.0, 2.6 Hz, 2H), 4.62 (td, J=10.7, 3.5 Hz, 2H), 4.13-4.01 (m, 4H), 3.97-3.87 (m, 8H), 3.85-3.75 (m, 2H), 3.74-3.63 (m, 2H), 3.31-3.22 (m, 4H), 3.14 (tdd, J=16.2, 10.8, 2.2 Hz, 2H), 2.73-2.56 (m, 4H), 2.38 (d, J=2.4 Hz, 3H), 2.02-1.92 (m, 4H), 1.73 (dd, J=9.4, 6.0 Hz, 2H), 1.04-0.90 (m, 4H), 0.05-−0.00 (m, 18H). MS (ES⁺) m/z (relative intensity) 1171.40 ([M+H]⁺, 100).

### (b) (9H-fluoren-9-yl)methyl ((S)-1-(((S)-1-((4-((S)-7-methoxy-8-((5-(((S)-7-methoxy-2-(4-(4-methylpiperazin-1-yl)phenyl)-5,11-dioxo-10-((2-(trimethylsilyl)ethoxy)methyl)-5,10,11,11a-tetrahydro-1H-benzo[e]pyrrolo[1,2-a][1,4]diazepin-8-yl)oxy)pentyl)oxy)-5, 11-dioxo-10-((2-(trimethylsilyl)ethoxy)methyl)-5,10, 11,11a-tetrahydro-1H-benzo[e]pyrrolo[1,2-a][1,4]diazepin-2-yl)phenyl)amino)-1-oxopropan-2-yl)amino)-3-methyl-1-oxobutan-2-yl)carbamate (89)

Pd(PPh₃)₄ (8 mg, 7 μmol) was added to a stirred mixture of the asymmetrical triflate 88 (269 mg, 0.23 mmol), Fmoc-Val-Ala-4-aminophenylboronic acid, pinacol ester 20 (210 mg, 0.34 mmol), Na₂CO₃ (36.5 mg, 0.34 mmol), EtOH (5 mL), toluene (10 mL), THF (1 mL), and water (5 mL). The reaction mixture was allowed to stir under a nitrogen atmosphere at 35° C. for 2 hours after which time the complete consumption of starting material was observed by TLC (80:20 v/v EtOAc/MeOH) and LC/MS (1.68 min (ES+) m/z (relative intensity) 1508.10 ([M+H]⁺, 100)). The reaction mixture was diluted with EtOAc (100 mL) and washed with H₂O (1×100 mL), brine (200 mL), dried (MgSO₄), filtered and evaporated under reduced pressure to provide the crude product. Purification by flash chromatography (gradient elution: 100:0 v/v EtOAc/MeOH to 80:20 v/v EtOAc/MeOH) afforded the SEM protected dimer 89 (240 mg, 69%). ¹H NMR (400 MHz, CDCl₃) δ 8.42 (s, 1H), 7.76 (d, J=7.5 Hz, 2H), 7.63-7.49 (m, 4H), 7.45-7.28 (m, 9H), 7.25 (d, J=2.9 Hz, 1H), 6.87 (t, J=14.0 Hz, 2H), 6.41 (s, 1H), 5.63-5.49 (m, 2H), 5.25 (s, 1H), 4.71 (d, J=10.1 Hz, 2H), 4.68-4.57 (m, 2H), 4.49 (d, J=6.7 Hz, 2H), 4.20 (s, 1H), 4.16-4.02 (m, 4H), 4.00-3.87 (m, 7H), 3.86-3.61 (m, 7H), 3.30-3.21 (m, 4H), 3.19-3.05 (m, 2H), 2.69-2.54 (m, 4H), 2.37 (s, 3H), 2.04-1.92 (m, 4H), 1.91-1.79 (m, 4H), 1.72 (s, 2H), 1.46 (d, J=6.9 Hz, 3H), 1.04-0.82 (m, 8H), 0.04-−0.02 (m, 18H). MS (ES⁺) m/z (relative intensity) 1508.10 ([M+H]⁺, 100).

### (c) (9H-fluoren-9-yl)methyl ((S)-1-(((S)-1-((4-((S)-7-methoxy-8-((5-(((S)-7-methoxy-2-(4-(4-methylpiperazin-1-yl)phenyl)-5-oxo-5,11a-dihydro-1H-benzo[e]pyrrolo[1,2-a][1,4]diazepin-8-yl)oxy)pentyl)oxy)-5-oxo-5,11a-dihydro-1H-benzo[e]pyrrolo[1,2-a][1,4]diazepin-2-yl)phenyl)amino)-1-oxopropan-2-yl)amino)-3-methyl-1-oxobutan-2-yl)carbamate (90)

Super hydride (0.358 mL, 0.358 mmol, 1.0 M in THF) was added dropwise to a stirred solution of the SEM-tetralactam 89 (216 mg, 0.143 mmol) in anhydrous THF (10 mL) at −78° C. The reaction mixture was allowed to stir for 3 hours after which time the complete conversion of starting material directly was observed by LC/MS (1.37 min (ES+) m/z (relative intensity) 608.15 (([M+2H]²⁺)/2, 100)). The reaction mixture was carefully diluted with H₂O (100 mL) and extracted with DCM (100 mL). The organic layers was washed with brine (100 mL), dried over MgSO₄, filtered and evaporated under reduced pressure to provide the intermediate SEM-carbinolamine. The white solids were immediately dissolved in MeOH (100 mL), DCM (10 mL) and H₂O (20 mL) and treated with flash silica gel (50 g). The thick suspension was allowed to stir at room temperature for 4 days after which time the formation of a significant quantity of desired product was observed by TLC (90:10 v/v CHCl₃/MeOH). The reaction mixture was filtered through a porosity 3 sinter funnel and the pad rinsed slowly and thoroughly with 90:10 v/v CHCl₃/MeOH until no further product eluted (checked by TLC). The filtrate was washed with brine (100 mL), dried (MgSO₄), filtered and evaporated in vacuo, followed by high vacuum drying, to provide the crude product. Purification by flash chromatography (gradient elution: HPLC grade 98:2 v/v CHCl₃/MeOH to 88:12 v/v CHCl₃/MeOH) gave 90 as a mixture of carbinolamine ethers and imine (80 mg, 46%).

¹H NMR (400 MHz, CDCl3) δ 8.52 (s, 1H), 7.87 (d, J=3.9 Hz, 2H), 7.75 (d, J=7.5 Hz, 2H), 7.66-7.26 (m, 12H), 6.90 (d, J=8.8 Hz, 2H), 6.81 (s, 1H), 6.64 (d, J=6.0 Hz, 1H), 5.37 (d, J=5.7 Hz, 1H), 4.74-4.58 (m, 2H), 4.54-4.31 (m, 4H), 4.26-3.98 (m, 6H), 3.94 (s, 2H), 3.86 (dd, J=13.6, 6.6 Hz, 1H), 3.63-3.48 (m, 2H), 3.37 (dd, J=16.5, 5.6 Hz, 2H), 3.31-3.17 (m, 4H), 2.66-2.51 (m, 4H), 2.36 (s, 3H), 2.16 (d, J=5.1 Hz, 1H), 2.06-1.88 (m, 4H), 1.78-1.55 (m, 6H), 1.46 (d, J=6.8 Hz, 3H), 0.94 (d, J=6.8 Hz, 6H). MS (ES⁺) m/z (relative intensity) 608.15 (([M+2H]²⁺)/2, 100).

### (d) 1-(3-(2,5-dioxo-2,5-dihydro-1H-pyrrol-1-yl)propanamido)-N—((S)-1-(((S)-1-((4-((S)-7-methoxy-8-((5-(((S)-7-methoxy-2-(4-(4-methylpiperazin-1-yl)phenyl)-5-oxo-5,11a-dihydro-1H-benzo[e]pyrrolo[1,2-a][1,4]diazepin-8-yl)oxy)pentyl)oxy)-5-oxo-5,11a-dihydro-1H-benzo[e]pyrrolo[1,2-a][1,4]diazepin-2-yl)phenyl)amino)-1-oxopropan-2-yl)amino)-3-methyl-1-oxobutan-2-yl)-3,6,9, 12,15, 18,21,24-octaoxaheptacosan-27-amide (91)

Piperidine (0.2 mL) was added to a solution of 90 (77 mg, 63.4 μmol) in DMF (1 mL). The reaction mixture was allowed to stir for 20 minutes. The reaction mixture was carefully diluted with DCM (50 mL) and washed with water (50 mL). The organic layers was washed with brine (100 mL), dried over MgSO₄, filtered and evaporated under reduced pressure to provide the unprotected valine intermediate. The crude residue was immediately redissolved in chloroform (5 mL). Mal(Peg)₈-acid (56 mg, 95 μmol) and EDCI (18 mg, 95 μmol) were added, followed by methanol (0.1 mL). The reaction was allowed to stir for 3 hours at room temperature at which point completion was observed by TLC and LC/IMS (1.19 min (ES+) m/z (relative intensity) 784.25 (([M+2H]²⁺)/2, 100)). The reaction mixture was diluted with chloroform (50 mL), washed with water (100 mL), dried (MgSO₄), filtered and evaporated in vacuo, followed by high vacuum drying, to provide the crude product. Purification by flash chromatography (gradient elution: HPLC grade 96:4 v/v CHCl₃/MeOH to 90:10 v/v CHCl₃/MeOH) gave 91 as a yellow solid (43 mg, 43%). ¹H NMR (400 MHz, CDCl₃) δ 8.73 (s, 1H), 7.88 (dd, J=7.6, 3.9 Hz, 2H), 7.75 (d, J=8.6 Hz, 2H), 7.52 (d, J=2.0 Hz, 2H), 7.44 (s, 1H), 7.40-7.28 (m, 4H), 6.91 (d, J=8.8 Hz, 2H), 6.81 (s, 2H), 6.69 (s, 2H), 6.48 (s, 1H), 4.72-4.63 (m, 1H), 4.46-4.34 (m, 2H), 4.25-4.03 (m, 6H), 3.95 (s, 4H), 3.84 (dd, J=17.2, 10.1 Hz, 4H), 3.72-3.46 (m, 30H), 3.44-3.32 (m, 4H), 3.30-3.20 (m, 4H), 2.75-2.63 (m, 1H), 2.59 (s, 4H), 2.55-2.43 (m, 3H), 2.37 (s, 3H), 2.29 (dd, J=12.7, 6.7 Hz, 1H), 2.03-1.89 (m, 4H), 1.72 (d, J=22.7 Hz, 8H), 1.46 (d, J=7.2 Hz, 3H), 1.01 (dd, J=11.5, 6.9 Hz, 6H). MS (ES⁺) m/z (relative intensity) 784.25 (([M+2H]²⁺)/2, 100).

### Example 4

### (i) (S)-((pentane-1,5-diylbis(oxy))bis(2-amino-5-methoxy-4,1-phenylene))bis(((S)-2-(((tert-butyldimethylsilyl)oxy)methyl)-4-methyl-2,3-dihydro-1H-pyrrol-1-yl)methanone) (98)

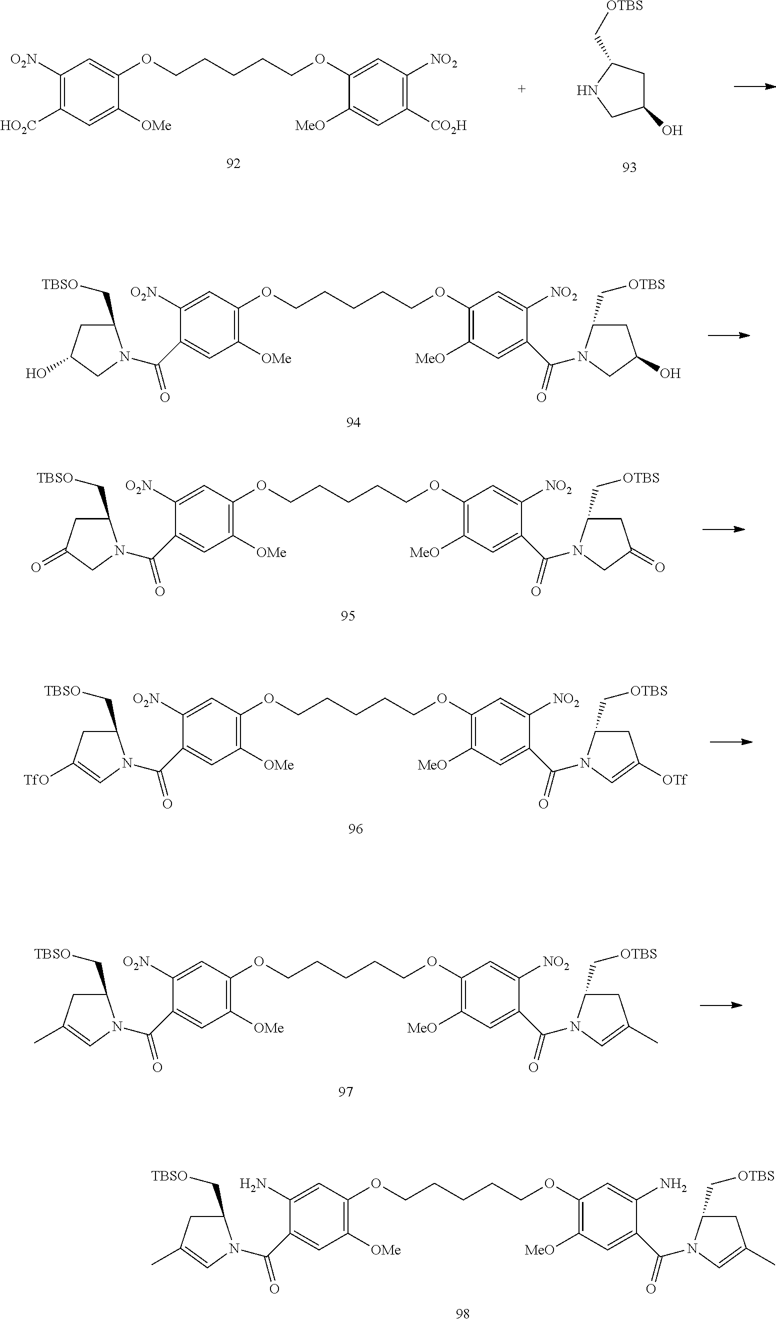

### (a) (S,R)-((pentane-1,5-diylbis(oxy))bis(5-methoxy-2-nitro-4,1-phenylene))bis(((2S,4R)-2-(((tert-butyldimethylsilyl)oxy)methyl)-4-hydroxypyrrolidin-1-yl)methanone) (94)

Anhydrous DMF (approx. 0.5 mL) was added dropwise to a stirred suspension of 4,4′-(pentane-1,5-diylbis(oxy))bis(5-methoxy-2-nitrobenzoic acid) (92) (36.64 g, 74.0 mmol) and oxalyl chloride (18.79 mL, 0.222 mol, 3.0 eq.) in anhydrous DCM (450 mL) until vigorous effervescence occurred and the reaction mixture was left to stir overnight. The reaction mixture was evaporated to dryness, and triturated with diethyl ether. The resulting yellow precipitate was filtered from solution, washed with diethyl ether (100 mL) and immediately added to a solution of (3R,5S)-5-((tert-butyldimethylsilyloxy)methyl) pyrrolidin-3-ol (93) (39.40 g, 0.170 mol, 2.3 eq.) and anhydrous triethylamine (82.63 mL, 0.592 mol, 8 eq.) in anhydrous DCM (400 mL) at −40° C. The reaction mixture was allowed to slowly warm to room temperature (over 2.5 hours) after which, LCMS analysis indicated complete reaction. DCM (250 mL) was added and the mixture was transferred into a separating funnel. The organic layer was washed successively with 0.1M HCl (2×800 mL), saturated NaHCO₃ (500 mL) and brine (300 mL). After drying over MgSO₄ and filtration, evaporation of the solvent left the product as a yellow foam (62.8 g, 92%). LC/MS: RT 1.96 min; MS (ES+) m/z (relative intensity) 921.45 ([M+H]⁺, 100).

### (b) (5S,5′S)-1,1′-(4,4′-(pentane-1,5-diylbis(oxy))bis(5-methoxy-2-nitrobenzoyl))bis(5-(((tert-butyldimethylsilyl)oxy)methyl)pyrrolidin-3-one) (95)

Trichloroisocyanuric acid (21.86 g, 94.07 mmol, 1.4 eq) was added in one portion to a solution of diol 94 (61.90 g, 67.20 mmol) and TEMPO (2.10 g, 13.44 mmol, 0.2 eq) in anhydrous DCM (500 mL) under an atmosphere of argon at 0° C. The reaction mixture was stirred at 0° C. for 20 minutes after which, LCMS analysis of the reaction mixture showed complete reaction. The reaction mixture was diluted with DCM (400 mL) and washed with saturated sodium bicarbonate (500 mL), 0.2 M sodium thiosulfate solution (600 mL), brine (400 mL) and dried (MgSO₄). Evaporation of the solvent gave the crude product. Flash chromatography [gradient elution 80% n-hexane/20% ethyl acetate to 100% ethyl acetate] gave pure 95 as yellow solid (49.30 g, 80%). LC/MS: RT 2.03 min; MS (ES+) m/z (relative intensity) 917.55 ([M+H]⁺, 100).

### (c) (5S,5'S)-1, 1′-(4, 4′-(pentane-1,5-diylbis(oxy))bis(5-methoxy-2-nitrobenzoyl))bis(5-(((tert-butyldimethylsilyl)oxy)methyl)-4,5-dihydro-1H-pyrrole-3, 1-diyl)bis(trifluoromethanesulfonate), (96)

Triflic anhydride (24.19 mL, 0.144 mol, 6.0 eq) was added dropwise to a vigorously stirred solution of bis-ketone 95 (21.98 g, 23.96 mmol) in anhydrous DCM (400 mL) containing 2,6-lutidine (22.33 mL, 0.192 mol, 8.0 eq) at −40° C. The reaction mixture was stirred at −40° C. for 30 min after which, LCMS analysis indicated complete reaction. Reaction mixture was rapidly diluted with DCM (500 mL) and washed with ice-cold water (600 mL), ice-cold saturated sodium bicarbonate (400 mL) and brine (500 mL), dried over MgSO₄, filtered and evaporated to leave a crude brown oil. Flash chromatography [gradient elution 80% n-hexane/20% ethyl acetate to 66% n-hexane/33% ethyl acetate] gave pure 96 as a brown foam (16.40 g, 58%). LC/MS: RT 2.28 min; MS (ES+) m/z (relative intensity) no data.

### (d) (S)-((pentane-1,5-diylbis(oxy))bis(5-methoxy-2-nitro-4, 1-phenylene))bis(((S)-2-(((tert-butyldimethylsilyl)oxy)methyl)-4-methyl-2,3-dihydro-1H-pyrrol-1-yl)methanone) (97)

Triflate 96 (5.06 g, 4.29 mmol), methyl boronic acid (1.80 g, 30.00 mmol, 7 eq) and triphenylarsine (1.05 g, 3.43 mmol, 0.8 eq) were dissolved in anhydrous dioxane and stirred under argon. Pd (II) bisbenzonitrile chloride was then added and the reaction mixture heated rapidly to 80° C. for 20 min. Reaction mixture cooled, filtered through Celite (washed through with ethyl acetate), filtrate washed with water (500 mL), brine (500 mL), dried over MgSO₄, filtered and evaporated. Flash chromatography [gradient elution 50% n-hexane/50% ethyl acetate] gave pure 97 as a brown foam (4.31 g, 59%). LC/MS: RT 2.23 min; MS (ES+) m/z (relative intensity) 913.50 ([M+H]⁺, 100).

### (e) (S)-((pentane-1,5-diylbis(oxy))bis(2-amino-5-methoxy-4, 1-phenylene))bis(((S)-2-(((tert-butyldimethylsilyl)oxy)methyl)-4-methyl-2,3-dihydro-1H-pyrrol-1-yl)methanone) (98)

Zinc dust (26.48 g, 0.405 mol, 36.0 eq) was added in one portion to a solution of bis-nitro compound 97 (10.26 g, 11.24 mmol) in 5% formic acid/methanol (200 mL) keeping the temperature between 25-30° C. with the aid of a cold water bath. The reaction was stirred at 30° C. for 20 minutes after which, LCMS showed complete reaction. The reaction mixture was filtered through Celite to remove the excess zinc, which was washed with ethyl acetate (600 mL). The organic fractions were washed with water (500 mL), saturated sodium bicarbonate (500 mL) and brine (400 mL), dried over MgSO₄ and evaporated. Flash chromatography [gradient elution 100% chloroform to 99% chloroform/1% methanol] gave pure 98 as an orange foam (6.22 g, 65%). LC/MS: RT 2.20 min; MS (ES+) m/z (relative intensity) 853.50 ([M+H]⁺, 100).

### (ii) 4-((R)-2-((R)-2-(((allyloxy)carbonyl)amino)-3-methylbutanamido)propanamido)benzyl 4-((10R,13R)-10-isopropyl-13-methyl-8,11-dioxo-2,5-dioxa-9,12-diazatetradecanamido)benzyl ((S)-(pentane-1,5-diylbis(oxy))bis(2-((S)-2-(hydroxymethyl)-4-methyl-2,3-dihydro-1H-pyrrole-1-carbonyl)-4-methoxy-5,1-phenylene))dicarbamate (103)

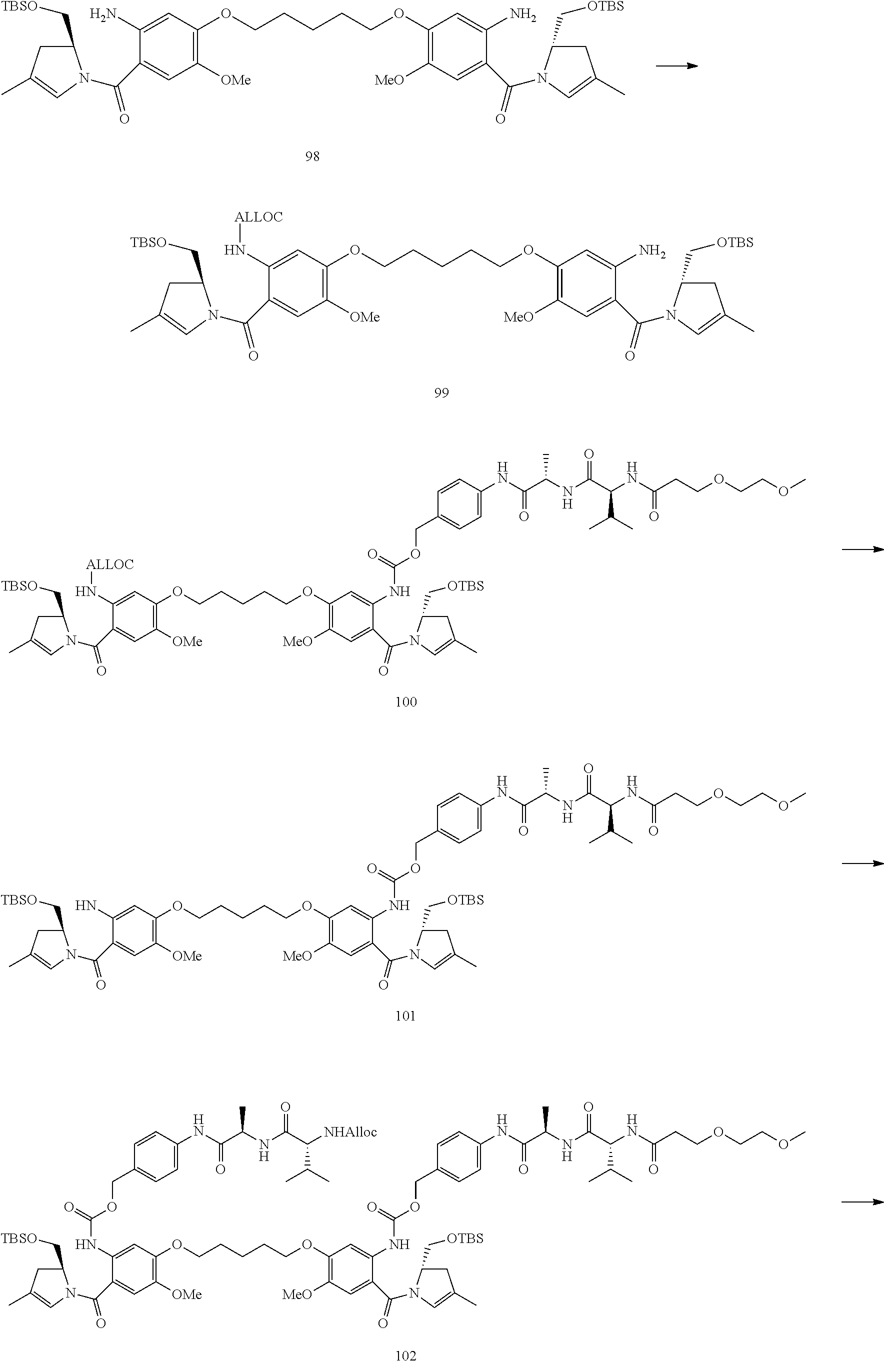

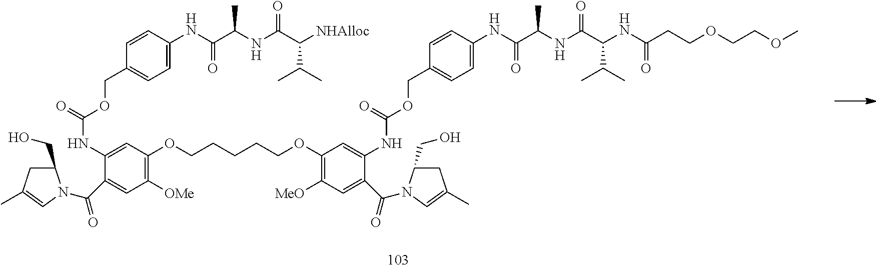

### (a) Allyl (5-((5-(5-amino-4-((S)-2-(((tert-butyldimethylsilyl)oxy)methyl)-4-methyl-2,3-dihydro-1H-pyrrole-1-carbonyl)-2-methoxyphenoxy)pentyl)oxy)-2-((S)-2-(((tert-butyldimethylsilyl)oxy)methyl)-4-methyl-2,3-dihydro-1H-pyrrole-1-carbonyl)-4-methoxyphenyl)carbamate (99)

Pyridine (1.156 mL, 14.30 mmol, 1.5 eq) was added to a solution of the bis-aniline 98 (8.14 g, 9.54 mmol) in anhydrous DCM (350 mL) at −78° C. under an atmosphere of argon. After 5 minutes, allyl chloroformate (0.911 mL, 8.58 mmol, 0.9 eq) was added and the reaction mixture allowed to warm to room temperature. The reaction mixture was diluted with DCM (250 mL), washed with saturated CuSO₄ solution (400 mL), saturated sodium bicarbonate (400 mL) and brine (400 mL), dried over MgSO₄. Flash chromatography [gradient elution 66% n-hexane/33% ethyl acetate to 33% n-hexane/66% ethyl acetate] gave pure 99 as an orange foam (3.88 g, 43%). LC/MS: RT 2.27 min; MS (ES+) m/z (relative intensity) 937.55 ([M+H]⁺, 100).

### (b) Allyl 4-((10S,13S)-10-isopropyl-13-methyl-8,11-dioxo-2,5-dioxa-9, 12-diazatetradecanamido)benzyl ((S)-(pentane-1,5-diylbis(oxy))bis(2-((S)-2-(((tert-butyldimethylsilyl)oxy)methyl)-4-methyl-2,3-dihydro-1H-pyrrole-1-carbonyl)-4-methoxy-5, 1-phenylene))dicarbamate (100)

Triethylamine (0.854 mL, 6.14 mmol, 2.2 eq) was added to a stirred solution of the aniline 99 (2.62 g, 2.79 mmol) and triphosgene (0.30 g, 1.00 mmol, 0.36 eq) in anhydrous THF (50 mL) under argon 0° C. The reaction mixture was stirred at room temperature for 5 minutes. LCMS analysis of an aliquot quenched with methanol, showed formation of the isocyanate. A solution of mPEG₂-Val-Ala-PAB-OH (1.54 g, 3.63 mmol, 1.3 eq) and triethylamine (0.583 mL, 4.19 mmol, 1.5 eq) in dry THF (50 mL) was added in one portion and the resulting mixture was stirred overnight at 40° C. The solvent of the reaction mixture was evaporated leaving a crude product. Flash chromatography [gradient elution 100% chloroform to 98% chloroform/2% methanol] gave pure 100 as a light orange solid (2.38 g, 62%). LC/MS: RT 2.29 min; MS (ES+) m/z (relative intensity) no data.

### (c) 4-((10S,13S)-10-isopropyl-13-methyl-8, 11-dioxo-2, 5-dioxa-9, 12-diazatetradecanamido)benzyl (5-((5-(5-amino-4-((S)-2-(((tert-butyldimethylsilyl)oxy)methyl)-4-methyl-2,3-dihydro-1H-pyrrole-1-carbonyl)-2-methoxyphenoxy)pentyl)oxy)-2-((S)-2-(((tert-butyldimethylsilyl)oxy)methyl)-4-methyl-2,3-dihydro-1H-pyrrole-1-carbonyl)-4-methoxyphenyl)carbamate (101)

Tetrakis(triphenylphosphine)palladium (39 mg, 0.034 mmol, 0.02 eq) was added to a stirred solution of 100 (2.35 g, 1.69 mmol) and pyrrolidine (0.35 mL, 4.24 mmol, 2.5 eq) in anhydrous DCM (25 mL) under argon at room temperature. Reaction mixture allowed to stir for 45 min then diluted with DCM (100 mL), washed with saturated ammonium chloride solution (100 mL), brine (100 mL), dried over MgSO₄, filtered and evaporated. Flash chromatography [gradient elution 100% chloroform to 95% chloroform/5% methanol] gave pure 101 as a yellow solid (1.81 g, 82%). LC/MS: RT 2.21 min; MS (ES+) m/z (relative intensity) 1303.65 ([M+H]⁺, 100).

### (d) 4-((R)-2-((R)-2-(((allyloxy)carbonyl)amino)-3-methylbutanamido)propanamido)benzyl 4-((10R,13R)-10-isopropyl-13-methyl-8, 11-dioxo-2,5-dioxa-9, 12-diazatetradecanamido)benzyl ((S)-(pentane-1,5-diylbis(oxy))bis(2-((S)-2-(((tert-butyldimethylsilyl)oxy)methyl)-4-methyl-2,3-dihydro-1H-pyrrole-1-carbonyl)-4-methoxy-5, 1-phenylene))dicarbamate (102)

Triethylamine (0.419 mL, 3.01 mmol, 2.2 eq) was added to a stirred solution of the aniline 101 (1.78 g, 1.37 mmol) and triphosgene (0.15 g, 0.49 mmol, 0.36 eq) in anhydrous THF (50 mL) under argon 0° C. The reaction mixture was stirred at room temperature for 5 min. LCMS analysis of an aliquot quenched with methanol, showed formation of the isocyanate. A solution of Alloc-Val-Ala-PAB-OH (0.67 g, 1.78 mmol, 1.3 eq) and triethylamine (0.29 mL, 2.05 mmol, 1.5 eq) in dry THF (45 mL) was added in one portion and the resulting mixture was stirred overnight at 40° C. The solvent of the reaction mixture was evaporated leaving a crude product. Flash chromatography [gradient elution 100% ethyl acetate to 97% ethyl acetate/3% methanol] gave pure 102 as a pale yellow solid (1.33 g, 57%).

LC/MS: RT 2.21 min; MS (ES+) m/z (relative intensity) no data.

### (e) 4-((R)-2-((R)-2-(((allyloxy)carbonyl)amino)-3-methylbutanamido)propanamido)benzyl 4-((10R,13R)-10-isopropyl-13-methyl-8, 11-dioxo-2,5-dioxa-9, 12-diazatetradecanamido)benzyl ((S)-(pentane-1,5-diylbis(oxy))bis(2-((S)-2-(hydroxymethyl)-4-methyl-2,3-dihydro-1H-pyrrole-1-carbonyl)-4-methoxy-5, 1-phenylene))dicarbamate (103)

Tetra-n-butylammonium fluoride (1 M, 1.52 mL, 1.52 mmol, 2.0 eq) was added to a solution of the TBS protected compound 102 (1.30 g, 0.76 mmol) in anhydrous THF (15 mL). The reaction mixture was stirred at room temperature for 4 hours. The reaction mixture was diluted with chloroform (100 mL) and washed sequentially with water (40 mL) and brine (40 mL). The organic phase was dried over MgSO₄ and evaporated to leave a yellow solid. Flash chromatography [gradient elution 95% ethyl acetate/5% methanol to 90% ethyl acetate/10% methanol] gave pure 103 as a pale yellow solid (1.00 g, 89%). LC/MS: RT 1.60 min; MS (ES+) m/z (relative intensity) 1478.45 (100).

### (iii) (11S,11aS)-4-((2R,5R)-37-(2,5-dioxo-25-dihydro-1H-pyrol-1-yl)-5-isopropyl-2-methyl-4,7,35-trioxo-10,13,16,19,22,25,28,31-octaoxa-3,6,34-triazaheptatriacontanamido)benzyl 11-hydroxy-8-((5-(((11S,11aS)-11-hydroxy-10-(((4-((10R,13R)-10-isopropyl-13-methyl-8,11-dioxo-2,5-dioxa-9,12-diazatetradecanamido)benzyl)oxy)carbonyl)-7-methoxy-2-methyl-5-oxo-5,10,11,11a-tetrahydro-1H-pyrrolo[2,1-c][1,4]benzodiazepin-8-yl)oxy)pentyl)oxy)-7-methoxy-2-methyl-5-oxo-11,11a-dihydro-1H-pyrrolo[2,1-c][1,4]benzodiazepine-10(5H)-carboxylate (106

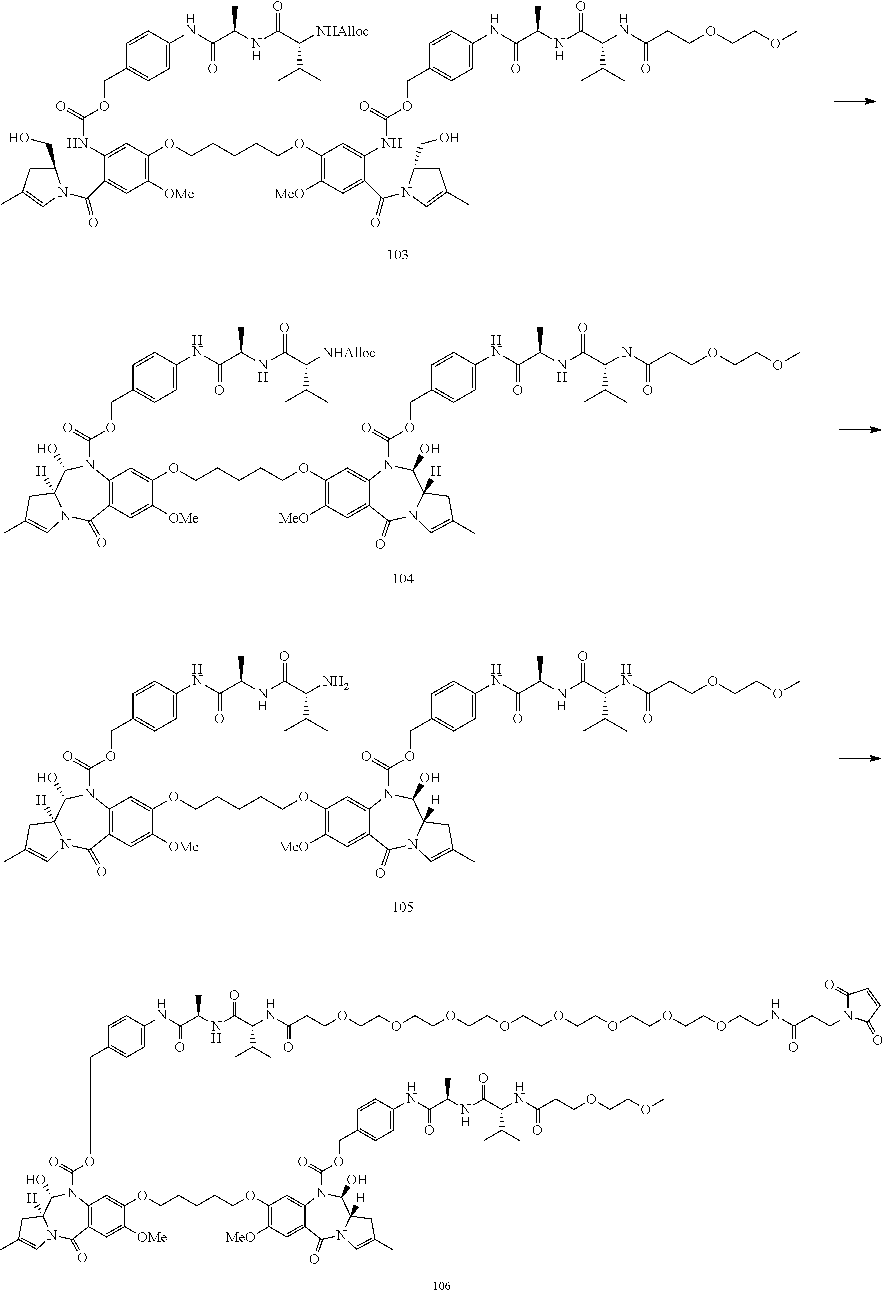

### (a) (11S,11aS)-4-((R)-2-((R)-2-(((allyloxy)carbonyl)amino)-3-methylbutanamido)propanamido)benzyl 11-hydroxy-8-((5-(((11S,11 aS)-11-hydroxy-10-(((4-((10R,13R)-10-isopropyl-13-methyl-8, 11-dioxo-2,5-dioxa-9, 12-diazatetradecanamido)benzyl)oxy)carbonyl)-7-methoxy-2-methyl-5-oxo-5,10,11,11a-tetrahydro-1H-pyrrolo[2, 1-c][1,4]benzodiazepin-8-yl)oxy)pentyl)oxy)-7-methoxy-2-methyl-5-oxo-11,11a-dihydro-1H-pyrrolo[2,1-c][1,4]benzodiazepine-10(5H)-carboxylate (104)

Dess-Martin periodinane (0.59 g, 1.38 mmol, 2.1 eq) was added to a stirred solution of 103 (0.97 g, 0.66 mmol) in anhydrous DCM under argon at room temperature. The reaction mixture was allowed to stir for 4 hours. Reaction mixture diluted with DCM (100 mL), washed with saturated sodium bicarbonate solution (3×100 mL), water (100 mL), brine (100 mL), dried over MgSO₄, filtered and evaporated. Flash chromatography [gradient elution 100% chloroform to 95% chloroform/5% methanol] gave pure 104 as a pale yellow solid (0.88 g, 90%). LC/MS: RT 1.57 min; MS (ES+) m/z (relative intensity) 1473.35 (100).

### (b) (11S,11aS)-4-((R)-2-((R)-2-amino-3-methylbutanamido)propanamido)benzyl 11-hydroxy-8-((5-(((11S,11 aS)-11-hydroxy-10-(((4-((10R,13R)-10-isopropyl-13-methyl-8, 11-dioxo-2,5-dioxa-9, 12-diazatetradecanamido)benzyl)oxy)carbonyl)-7-methoxy-2-methyl-5-oxo-5,10,11,11a-tetrahydro-1H-pyrrolo[2,1-c][1,4]benzodiazepin-8-yl)oxy)pentyl)oxy)-7-methoxy-2-methyl-5-oxo-11,11a-dihydro-1H-pyrrolo[2, 1-c][1,4]benzodiazepine-10(5H)-carboxylate (105)

Tetrakis(triphenylphosphine)palladium (5 mg, 0.004 mmol, 0.06 eq) was added to a solution of 104 (105 mg, 0.071 mmol) and pyrrolidine (7 μL, 0.086 mmol, 1.2 eq) in anhydrous DCM (5 mL). The reaction mixture was stirred 15 minutes then diluted with chloroform (50 mL) and washed sequentially with saturated aqueous ammonium chloride (30 mL) and brine (30 mL). The organic phase was dried over magnesium sulphate, filtered and evaporated. Flash chromatography [gradient elution 100% chloroform to 90% chloroform/10% methanol] gave pure 105 as a pale yellow solid (54 mg, 55%). LC/MS: RT 1.21 min; MS (ES+) m/z (relative intensity) 1389.50 (100).

### (c) (11S,11aS)-4-((2R,5R)-37-(2,5-dioxo-2,5-dihydro-1H-pyrrol-1-yl)-5-isopropyl-2-methyl-4,7,35-trioxo-10,13, 16, 19,22,25,28,31-octaoxa-3,6,34-triazaheptatriacontanamido)benzyl 11-hydroxy-8-((5-(((11S,11aS)-11-hydroxy-10-(((4-((10R,13R)-10-isopropyl-13-methyl-8,11-dioxo-2,5-dioxa-9, 12-diazatetradecanamido)benzyl)oxy)carbonyl)-7-methoxy-2-methyl-5-oxo-5,10,11,11a-tetrahydro-1H-pyrrolo[2, 1-c][1,4]benzodiazepin-8-yl)oxy)pentyl)oxy)-7-methoxy-2-methyl-5-oxo-11,11a-dihydro-1H-pyrrolo[2,1-c][1,4]benzodiazepine-10(5H)-carboxylate (106)

N-(3-Dimethylaminopropyl)-N′-ethylcarbodiimide (28 mg, 0.146 mmol, 1 eq) was added to a solution of 105 (203 mg, 0.146 mmol) and maleimide-PEG₈ acid (87 mg, 0.146 mmol) in chloroform (5 mL). The reaction was stirred for 1.5 h then diluted with chloroform (50 mL), washed with water (50 mL), brine (30 mL), dried over magnesium sulphate, filtered and evaporated. Flash chromatography [gradient elution 100% DCM to 90% DCM/10% methanol] gave 106 as a pale yellow solid (205 mg, 72%). LC/MS: RT 5.75 min; MS (ES+) m/z (relative intensity) 982.90 (100), 1963.70 (5).

### Example 5: Activity of Released Compounds

### K562 Assay

K562 human chronic myeloid leukaemia cells were maintained in RPM1 1640 medium supplemented with 10% fetal calf serum and 2 mM glutamine at 37° C. in a humidified atmosphere containing 5% CO₂ and were incubated with a specified dose of drug for 1 hour or 96 hours at 37° C. in the dark. The incubation was terminated by centrifugation (5 min, 300 g) and the cells were washed once with drug-free medium. Following the appropriate drug treatment, the cells were transferred to 96-well microtiter plates (10⁴ cells per well, 8 wells per sample). Plates were then kept in the dark at 37° C. in a humidified atmosphere containing 5% CO₂. The assay is based on the ability of viable cells to reduce a yellow soluble tetrazolium salt, 3-(4,5-dimethylthiazol-2-yl)-2,5-diphenyl-2H-tetrazolium bromide (MTT, Aldrich-Sigma), to an insoluble purple formazan precipitate. Following incubation of the plates for 4 days (to allow control cells to increase in number by approximately 10 fold), 20 μL of MTT solution (5 mg/mL in phosphate-buffered saline) was added to each well and the plates further incubated for 5 h. The plates were then centrifuged for 5 min at 300 g and the bulk of the medium pipetted from the cell pellet leaving 10⁻²⁰ μL per well. DMSO (200 μL) was added to each well and the samples agitated to ensure complete mixing. The optical density was then read at a wavelength of 550 nm on a Titertek Multiscan ELISA plate reader, and a dose-response curve was constructed. For each curve, an IC₅₀ value was read as the dose required to reduce the final optical density to 50% of the control value.

### Example 6: Formation of Conjugates

### General Antibody Conjugation Procedure

Antibodies between 1-10 mg/ml in 30 mM Histidine, 200 mM sorbitol, pH 6 is increased in pH from 6 to 7.5 by the addition of a volume of 0.5 M Tris, 25 mM EDTA, pH 8.5, equivalent to 6.7% of the mAb volume. DTT reductant is added to the batch as a 20-fold molar excess with respect to antibody and the reduction reaction is allowed to proceed overnight at room temperature (no agitation). Post reduction the antibody is desalted into 0.1 M sodium phosphate pH 7.5. Reduced antibody is reoxidised by the addition of 25 mM DHAA as a 20 fold molar excess with respect to antibody and the reoxidation reaction is allowed to proceed for a total of 2 hours at 20° C. Conjugation is initiated by the addition of DMA and 10 mM drug-linker is added in that order to achieve a 5% v/v final and 3 fold excess relative to the antibody, respectively. The conjugation reaction is incubated for 60 min. Post conjugation the reaction is quenched with a 3 fold molar excess of N-acetyl cysteine and incubated for an additional 30 mins. The final products can be analysed by SEC, HIC, PLRP and non-reducing gel electrophoresis.

Corresponding antibody-drug conjugates can be determined by analysis by High-Performance Liquid Chromatography (HPLC) or Ultra-High-Performance Liquid Chromatography (UHPLC) to assess drug-per-antibody ratio (DAR) using reverse-phase chromatography (RP) or Hydrophobic-Interaction Chromatography (HIC), coupled with UV-Visible, Fluorescence or Mass-Spectrometer detection; aggregate level and monomer purity can be analysed by HPLC or UHPLC using size-exclusion chromatography coupled with UV-Visible, Fluorescence or Mass-Spectrometer detection. Final conjugate concentration is determined by a combination of spectroscopic (absorbance at 280, 214 and 330 nm) and biochemical assay (bicinchonic acid assay BCA; Smith, P. K., et al. (1985) Anal. Biochem. 150 (1): 76-85; using a known-concentration IgG antibody as reference). Antibody-drug conjugates are generally sterile filtered using 0.2 m filters under aseptic conditions, and stored at +4° C., −20° C. or −80° C.

### DAR Determination

Antibody or ADC (ca. 35 pg in 35 μL) was reduced by addition of 10 μL borate buffer (100 mM, pH 8.4) and 5 μL DTT (0.5 M in water), and heated at 37° C. for 15 minutes. The sample was diluted with 1 volume of acetonitrile: water: formic acid (49%: 49%: 2% v/v), and injected onto a Widepore 3.6μ XB-C18 150×2.1 mm (P/N 00F-4482-AN) column (Phenomenex Aeris) at 80° C., in a UPLC system (Shimadzu Nexera) with a flow rate of 1 ml/min equilibrated in 75% Buffer A (Water, Trifluoroacetic acid (0.1% v/v) (TFA), 25% buffer B (Acetonitrile: water: TFA 90%: 10%: 0.1% v/v). Bound material was eluted using a gradient from 25% to 55% buffer B in 10 min. Peaks of UV absorption at 214 nm were integrated. The following peaks were identified for each ADC or antibody: native antibody light chain (L0), native antibody heavy chain (HO), and each of these chains with added drug-linkers (labelled L1 for light chain with one drug and H1, H2, H3 for heavy chain with 1, 2 or 3 attached drug-linkers). The UV chromatogram at 330 nm was used for identification of fragments containing drug-linkers (i.e., L1, H1, H2, H3).

A PBD/protein molar ratio was calculated for both light chains and heavy chains:

$$\frac{Drug}{Protein} ratio on light chain = \frac{\% Area at 214 nm for L 1}{\% Area at 214 nm for L 0 and L 1}$$

$$\frac{Drug}{Protein} ratio on heavy chain = \frac{{\sum }_{n = 0}^{3} n \times ( \% area at 214 for Hn )}{{\sum }_{n == 0}^{3} \% area at 214 for Hn}$$

Final DAR is Calculated as:

$$DAR = 2 \times ( \frac{Drug}{Protein} ratio on light chain + \frac{Drug}{Protein} ratio on heavy chain )$$

DAR measurement is carried out at 214 nm because it minimises interference from drug-linker absorbance.

### Abbreviations

5 Å+ set The FW residues in the 5A CDR envelope, defined by the homology model, together with the canonical, vernier and VH/VK interface residues
1H12 The anti-AXL mouse monoclonal antibody
1H12 VK VK of mouse 1H12 antibody
1H12RKA1 Humanised version, A1, of 1H12 VK
1H12RHA Humanised version, A, of 1H12 VH
1H12RHA×IgG1k antibody comprising the VH and VK constructs 1H12RHA and 1H12RKA 1H12RKA respectively, functionally contiguous with the constant regions of human IgG1 and Ig-kappa heavy and light chains respectively
A Adenine
A Angstrom
Ac acetyl
Acm acetamidomethyl
Alloc allyloxycarbonyl
B7 The anti-LPA antibody product of mouse hybridoma clone B7
Boc di-tert-butyl dicarbonate
bp base pairs
Bzl benzyl, where Bzl-OMe is methoxybenzyl and Bzl-Me is methylbenzene
C Cytosine
Cbz or Z benzyloxy-carbonyl, where Z—Cl and Z—Br are chloro- and bromobenzyloxy carbonyl respectively
CDR Complementarity determining region in the immunoglobulin variable regions, defined using the Kabat numbering system
CHO Chinese hamster ovary cell line
D-gene Diversity gene
DMF N,N-dimethylformamide
DNA Deoxyribonucleic acid
Dnp dinitrophenyl
DTT dithiothreitol
Fmoc 9H-fluoren-9-ylmethoxycarbonyl
FW Framework region: the immunoglobulin variable regions excluding the CDR regions
G Guanine
IgG Immunoglobulin G
imp N-10 imine protecting group: 3-(2-methoxyethoxy)propanoate-Val-Ala-PAB
MC-OSu maleimidocaproyl-O—N-succinimide
J-gene Joining gene
Kabat an immunoglobulin alignment and numbering system pioneered by Elvin A Kabat
mAb monoclonal antibody
Moc methoxycarbonyl
MP maleimidopropanamide
Mtr 4-methoxy-2,3,6-trimethtylbenzenesulfonyl
PAB para-aminobenzyloxycarbonyl
PEG ethyleneoxy
PNZ p-nitrobenzyl carbamate
Psec 2-(phenylsulfonyl)ethoxycarbonyl
T Thymine
TBDMS tert-butyldimethylsilyl
TBDPS tert-butyldiphenylsilyl
t-Bu tert-butyl
Teoc 2-(trimethylsilyl)ethoxycarbonyl
Tos tosyl
Troc 2,2,2-trichlorethoxycarbonyl chloride
Trt trityl
V region The segment of IgG chains which is variable in sequence between different antibodies. It extends to Kabat residue 109 in the light chain and 113 in the heavy chain.
VCI Framework residue classified as vernier or canonical or VH-VL interface
V-gene The gene segment that is rearranged, together with a J (and D for VH) gene, to generate a complete VK (or VH)
VH Immunoglobulin heavy chain variable region
VK Immunoglobulin kappa light chain variable region
Xan xanthyl

### REFERENCES

[1] C. Chothia, et al., “Domain association in immunoglobulin molecules. The packing of variable domains,” J Mol. Biol. 186(3), 651 (1985).
[2] J. Foote and G. Winter, “Antibody framework residues affecting the conformation of the hypervariable loops,” J Mol. Biol. 224(2), 487 (1992).
[3] E. A Kabat, et al., sequences of proteins of immunological interest, 5 ed. (NIH National Technical Information Service, 1991).
[4] V. Morea, A. M. Lesk, and A. Tramontano, “Antibody modeling: implications for engineering and design,” Methods 20(3), 267 (2000).

### Statements of Disclosure

1. An isolated humanized antibody that binds to AXL, wherein the isolated humanized antibody comprises a heavy chain variable region having the amino acid sequence of SEQ ID NO: 1, SEQ ID NO: 2 or SEQ ID NO: 3.

2. The isolated humanized antibody according to statement 1, wherein the isolated humanized antibody further comprises a light chain variable region having the amino acid sequence of SEQ ID NO: 4, 5, 6, 7, or 8; and, optionally, comprises a constant region derived from one or more human antibodies.

3. The isolated humanized antibody according to either one of statements 1 or 2, wherein the isolated humanized antibody comprises:
(i) a heavy chain variable region having the amino acid sequence of SEQ ID NO: 1 and a light chain variable region having the amino acid sequence of SEQ ID NO: 4;
(ii) a heavy chain variable region having the amino acid sequence of SEQ ID NO: 1 and a light chain variable region having the amino acid sequence of SEQ ID NO: 5;
(iii) a heavy chain variable region having the amino acid sequence of SEQ ID NO: 1 and a light chain variable region having the amino acid sequence of SEQ ID NO: 6;
(iv) a heavy chain variable region having the amino acid sequence of SEQ ID NO: 1 and a light chain variable region having the amino acid sequence of SEQ ID NO: 7;
(v) a heavy chain variable region having the amino acid sequence of SEQ ID NO: 1 and a light chain variable region having the amino acid sequence of SEQ ID NO: 8;
(vi) a heavy chain variable region having the amino acid sequence of SEQ ID NO: 2 and a light chain variable region having the amino acid sequence of SEQ ID NO: 4;
(vii) a heavy chain variable region having the amino acid sequence of SEQ ID NO: 2 and a light chain variable region having the amino acid sequence of SEQ ID NO: 5;
(viii) a heavy chain variable region having the amino acid sequence of SEQ ID NO: 2 and a light chain variable region having the amino acid sequence of SEQ ID NO: 6;
(ix) a heavy chain variable region having the amino acid sequence of SEQ ID NO: 2 and a light chain variable region having the amino acid sequence of SEQ ID NO: 7;
(x) a heavy chain variable region having the amino acid sequence of SEQ ID NO: 2 and a light chain variable region having the amino acid sequence of SEQ ID NO: 8;
(xi) a heavy chain variable region having the amino acid sequence of SEQ ID NO: 3 and a light chain variable region having the amino acid sequence of SEQ ID NO: 4;
(xii) a heavy chain variable region having the amino acid sequence of SEQ ID NO: 3 and a light chain variable region having the amino acid sequence of SEQ ID NO: 5;
(xiii) a heavy chain variable region having the amino acid sequence of SEQ ID NO: 3 and a light chain variable region having the amino acid sequence of SEQ ID NO: 6;
(xiv) a heavy chain variable region having the amino acid sequence of SEQ ID NO: 3 and a light chain variable region having the amino acid sequence of SEQ ID NO: 7; or
(xv) a heavy chain variable region having the amino acid sequence of SEQ ID NO: 3 and a light chain variable region having the amino acid sequence of SEQ ID NO: 8.

4. The humanized antibody according to any one of statements 1 to 3, wherein said antibody binds human AXL with an affinity (Kd) of at least 10⁻⁶ M, such as an affinity (Kd) of at least 10⁻⁹ M.

5. The humanized antibody according to any one of statements 1 to 4, wherein said antibody competitively inhibits the binding to human AXL of an antibody comprising a heavy chain variable region having the amino acid sequence of SEQ ID NO: 1 and a light chain variable region having the amino acid sequence of SEQ ID NO: 4.

6. The humanized antibody according to any one of statements 1 to 5, wherein said antibody:
(i) binds the Axl-Strep-His antigen with an affinity (Kd) of no more 0.6 of the Kd of an antibody comprising a VH domain having the sequence according to SEQ ID NO. 1, a VL domain having the sequence according to SEQ ID NO. 4, and a constant region derived from one or more human antibodies; or
(ii) binds the Axl-Fc antigen with an affinity (Kd) of no more 0.5 of the Kd of an antibody comprising a VH domain having the sequence according to SEQ ID NO. 1, a VL domain having the sequence according to SEQ ID NO. 4, and a constant region derived from one or more human antibodies.

7. The humanized antibody according to any one of statements 1 to 6, wherein said antibody competitively inhibits the binding to human AXL of the mouse 1H12 antibody.

8. The humanized antibody according to any one of statements 1 to 7, wherein said antibody has a pI of at least 8.00.

9. The humanized antibody according to statement 8 wherein the antibody has a pI of at least 8.15.

10. The humanized antibody according to any one of statements 1 to 9, wherein said antibody or antibody fragment has a constant region of either isotype IgG1, IgG2, IgG3 or IgG4, or a mutated IgG constant region, and optionally a light chain constant region of isotype kappa or lambda.

11. The humanized antibody according to any one of statements 1 to 10, wherein the humanized antibody fragment is a scFv, Fab or F(ab′)₂.

12. A conjugate of formula L-(DL)p, where DL is of formula I or II::

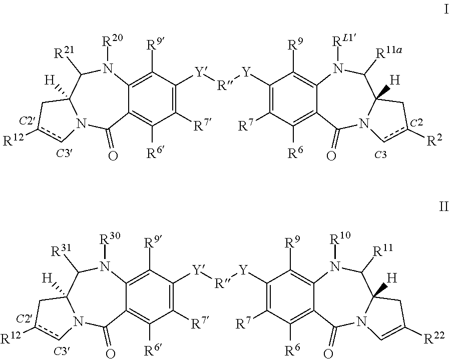

wherein:

L is an antibody (Ab) according to any one of statements 1 to 11;

when there is a double bond present between C2′ and C3′, R¹² is selected from the group consisting of:

(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;

(ib) C₁₋₅ saturated aliphatic alkyl;

(ic) C₃₋₆ saturated cycloalkyl;

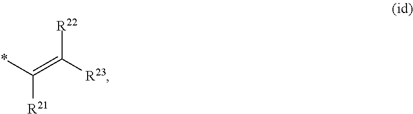

wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5;

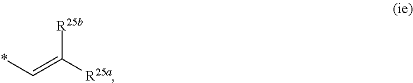

wherein one of R^{25a} and R^{25b} is H and the other is selected from: phenyl, which phenyl is optionally substituted by a group selected from halo, methyl, methoxy; pyridyl; and thiophenyl; and

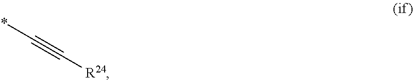

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;

when there is a single bond present between C2′ and C3′,

R¹² is

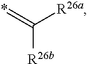

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;

R⁶ and R⁹ are independently selected from H, R, OH, OR, SH, SR, NH₂, NHR, NRR′, nitro, Me₃Sn and halo;

where R and R′ are independently selected from optionally substituted C₁₋₁₂ alkyl, C₃₋₂₀ heterocyclyl and C₅₋₂₀ aryl groups;

R⁷ is selected from H, R, OH, OR, SH, SR, NH₂, NHR, NHRR′, nitro, Me₃Sn and halo;

R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;

Y and Y′ are selected from O, S, or NH;

R^{6′}, R^{7′}, R^{9′} are selected from the same groups as R⁶, R⁷ and R⁹ respectively;

[Formula I]

R^{L1′} is a linker for connection to the antibody (Ab);

R^{11a} is selected from OH, OR^{A}, where R^{A} is C₁₋₄ alkyl, and SO_{z}M, where z is 2 or 3 and M is a monovalent pharmaceutically acceptable cation;

R²⁰ and R²¹ either together form a double bond between the nitrogen and carbon atoms to which they are bound or;

R²⁰ is selected from H and R^{C}, where R^{C} is a capping group;

R²¹ is selected from OH, OR^{A} and SO_{z}M;

when there is a double bond present between C2 and C3, R² is selected from the group consisting of:

(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;

(ib) C₁₋₅ saturated aliphatic alkyl;

(ic) C₃₋₆ saturated cycloalkyl;

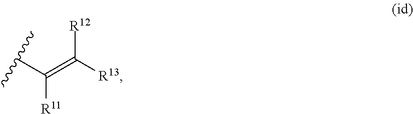

wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;

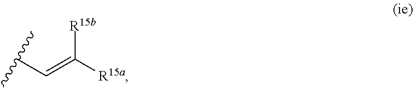

wherein one of R^{15a} and R^{15b} is H and the other is selected from: phenyl, which phenyl is optionally substituted by a group selected from halo, methyl, methoxy; pyridyl; and thiophenyl; and

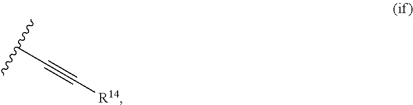

where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;

when there is a single bond present between C2 and C3,

R² is

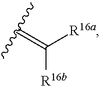

where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;

[Formula II]

R²² is of formula IIIa, formula IIIb or formula IIIc:

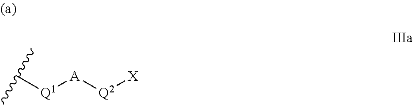

where A is a C₅₋₇ aryl group, and either

(i) Q¹ is a single bond, and Q² is selected from a single bond and —Z—(CH₂)ₙ—, where Z is selected from a single bond, O, S and NH and n is from 1 to 3; or

(ii) Q¹ is —CH═CH—, and Q² is a single bond;

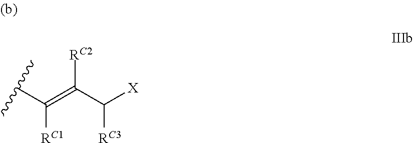

where;

R^{C1}, R^{C2} and R^{C3} are independently selected from H and unsubstituted C₁₋₂ alkyl;

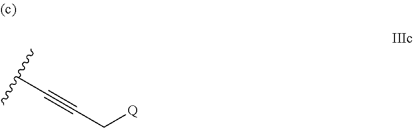

where Q is selected from O—R^{L2′}, S—R^{L2′} and NR^{N}—R^{L2′}, and R^{N} is selected from H, methyl and ethyl

X is selected from the group comprising: O—R^{L2′}, S—R^{L2′}, CO₂—R^{L2′}, CO—R^{L2′}, NH—C(═O)—R^{L2′}, NHNH—R^{L2′}, CONHNH—R^{L2′},

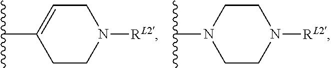

NR^{N}R^{L2′}, wherein R^{N} is selected from the group comprising H and C₁₋₄ alkyl;

R^{L2′} is a linker for connection to the antibody (Ab);

R¹⁰ and R¹¹ either together form a double bond between the nitrogen and carbon atoms to which they are bound or;

R¹⁰ is H and R¹¹ is selected from OH, OR^{A} and SO_{z}M;

R³⁰ and R³¹ either together form a double bond between the nitrogen and carbon atoms to which they are bound or;

R³⁰ is H and R³¹ is selected from OH, OR^{A} and SO_{z}M.

13. The conjugate according to statement 12, wherein the conjugate is not:

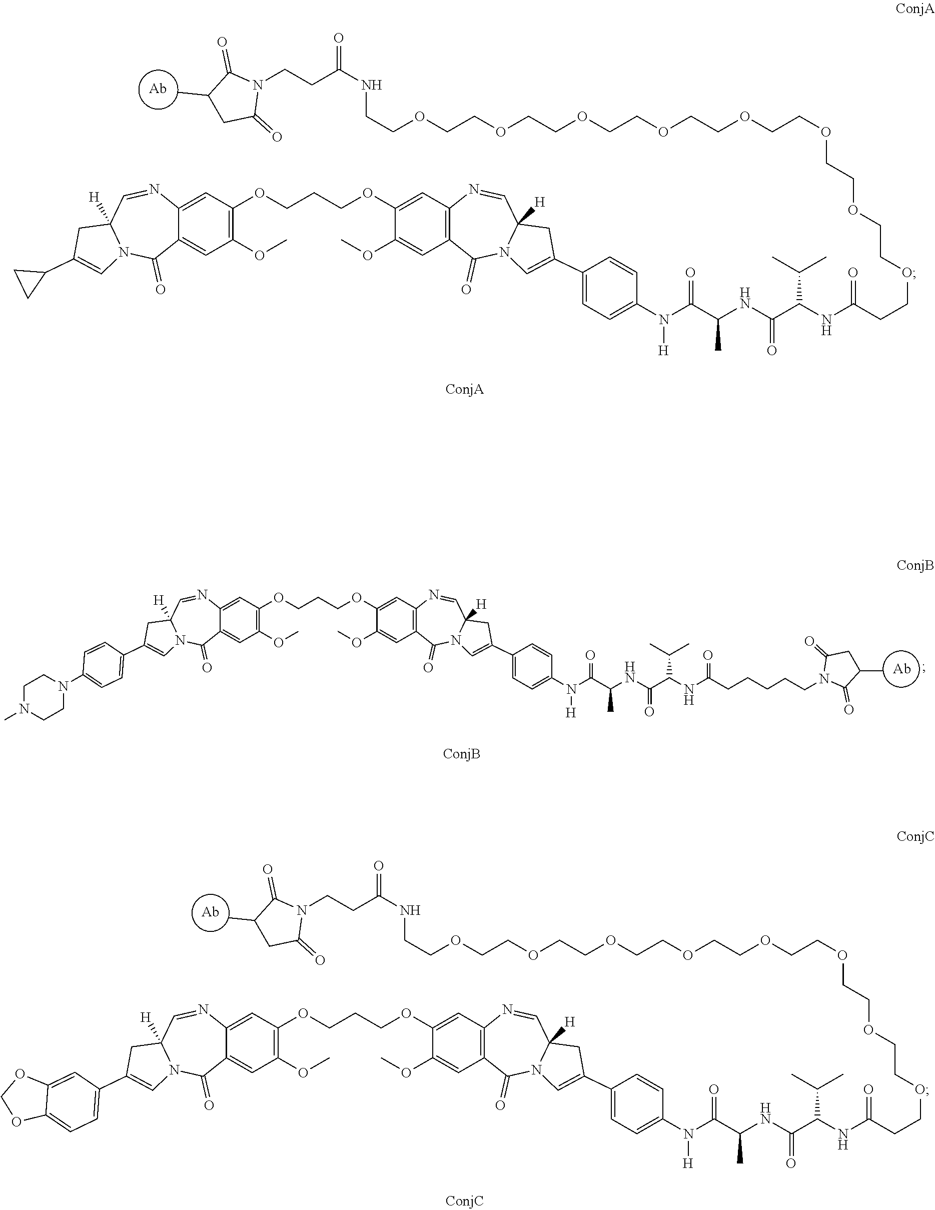

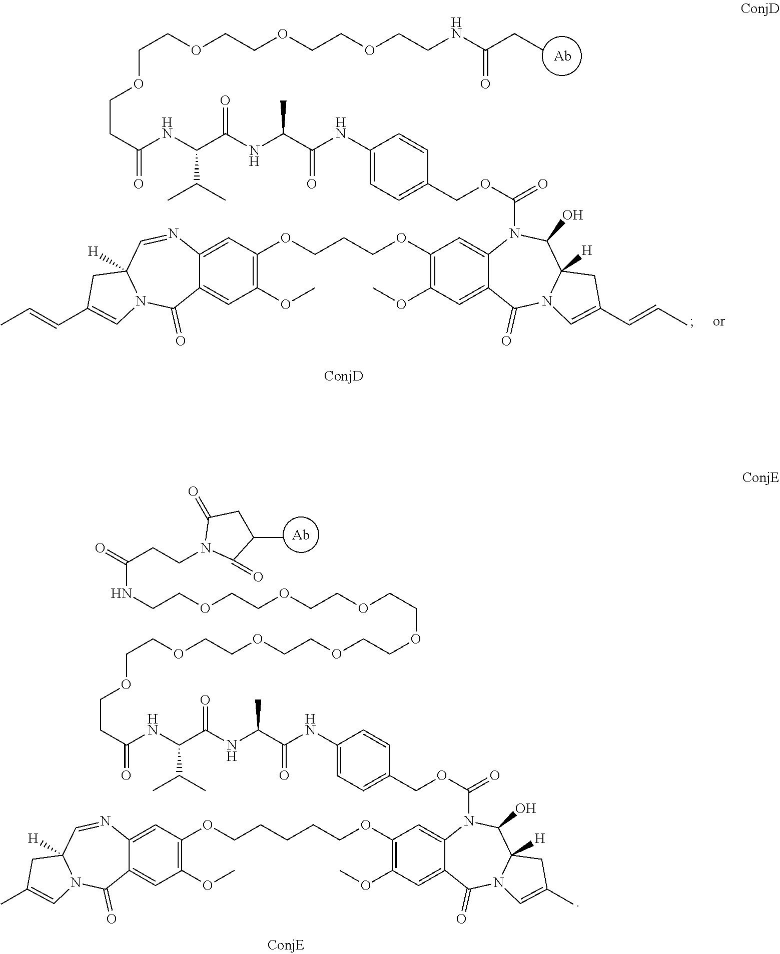

14. The conjugate according to either statement 12 or statement 13, wherein R⁷ is selected from H, OH and OR.

15. The conjugate according to statement 14, wherein R⁷ is a C₁₋₄ alkyloxy group.

16. The conjugate according to any one of statements 12 to 15, wherein Y is O.

17. The conjugate according to any one of the preceding statements, wherein R″ is C₃₋₇ alkylene.

18. The conjugate according to any one of statements 12 to 17, wherein R⁹ is H.

19. The conjugate according to any one of statements 12 to 18, wherein R⁶ is selected from H and halo.

20. The conjugate according to any one of statements 12 to 19, wherein there is a double bond between C2′ and C3′, and R¹² is a C₅₋₇ aryl group.

21. The conjugate according to statement 20, wherein R¹² is phenyl.

22. The conjugate according to any one of statements 12 to 19, wherein there is a double bond between C2′ and C3′, and R¹² is a C₈₋₁₀ aryl group.

23. The conjugate according to any one of statements 20 to 22, wherein R¹² bears one to three substituent groups.

24. The conjugate according to any one of statements 20 to 23, wherein the substituents are selected from methoxy, ethoxy, fluoro, chloro, cyano, bis-oxy-methylene, methyl-piperazinyl, morpholino and methyl-thiophenyl.

25. The conjugate according to any one of statements 12 to 19, wherein there is a double bond between C2′ and C3′, and R¹² is a C₁₋₅ saturated aliphatic alkyl group.

26. A compound according to statement 25, wherein R¹² is methyl, ethyl or propyl.

27. The conjugate according to any one of statements 12 to 19, wherein there is a double bond between C2′ and C3′, and R¹² is a C₃₋₆ saturated cycloalkyl group.

28. The conjugate according to statement 27, wherein R¹² is cyclopropyl.

29. The conjugate according to any one of statements 12 to 19, wherein there is a double bond between C2′ and C3′, and R¹² is a group of formula:

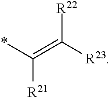

30. The conjugate according to statement 29, wherein the total number of carbon atoms in the R¹² group is no more than 4.

31. The conjugate according to statement 30, wherein the total number of carbon atoms in the R¹² group is no more than 3.

32. The conjugate according to any one of statements 29 to 31, wherein one of R²¹, R²² and R²³ is H, with the other two groups being selected from H, C₁₋₃ saturated alkyl, C₂₋₃ alkenyl, C₂₋₃ alkynyl and cyclopropyl.

33. The conjugate according to any one of statements 29 to 31, wherein two of R²¹, R²² and R²³ are H, with the other group being selected from H, C₁₋₃ saturated alkyl, C₂₋₃ alkenyl, C₂₋₃ alkynyl and cyclopropyl.

34. The conjugate according to any one of statements 12 to 19, wherein there is a double bond between C2′ and C3′, and R¹² is a group of formula:

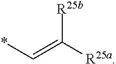

35. The conjugate according to statement 34, wherein R¹² is the group:

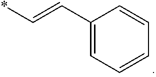 [SMILES: */C=C/c1ccccc1]

36. The conjugate according to any one of statements 12 to 19, wherein there is a double bond between C2′ and C3′, and R¹² is a group of formula:

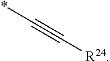

37. The conjugate according to statement 36, wherein R²⁴ is selected from H, methyl, ethyl, ethenyl and ethynyl.

38. The conjugate according to statement 37, wherein R²⁴ is selected from H and methyl.

39. The conjugate according to any one of statements 12 to 19, wherein there is a single bond between C2′ and C3′, R¹² is

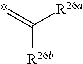

and R^{26a} and R^{26b} are both H.

40. The conjugate according to any one of statements 23 to 30, wherein there is a single bond between C2′ and C3′, R¹² is

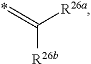

and R^{26a} and R^{26b} are both methyl.

41. The conjugate according to any one of statements 12 to 19, wherein there is a single bond between C2′ and C3′, R¹² is

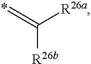

one of R^{26a} and R^{26b} is H, and the other is selected from C₁₋₄ saturated alkyl, C₂₋₃ alkenyl, which alkyl and alkenyl groups are optionally substituted.

[Formula I]

42. The conjugate according to any one of statements 12 to 41, wherein there is a double bond between C2 and C3, and R² is a C₅₋₇ aryl group.

43. The conjugate according to statement 42, wherein R² is phenyl.

44. The conjugate according to any one of statements 12 to 41, wherein there is a double bond between C2 and C3, and R¹ is a C₈₋₁₀ aryl group.

45. A compound according to any one of statements 42 to 44 wherein R² bears one to three substituent groups.

46. The conjugate according to any one of statements 42 to 45, wherein the substituents are selected from methoxy, ethoxy, fluoro, chloro, cyano, bis-oxy-methylene, methyl-piperazinyl, morpholino and methyl-thiophenyl.

47. The conjugate according to any one of statements 12 to 41, wherein there is a double bond between C2 and C3, and R² is a C₁₋₅ saturated aliphatic alkyl group.

48. The conjugate according to statement 47, wherein R² is methyl, ethyl or propyl.

49. The conjugate according to any one of statements 12 to 41, wherein there is a double bond between C2 and C3, and R² is a C₃₋₆ saturated cycloalkyl group.

50. The conjugate according to statement 49, wherein R² is cyclopropyl.

51. The conjugate according to any one of statements 12 to 41, wherein there is a double bond between C2 and C3, and R² is a group of formula:

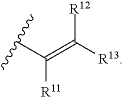

52. The conjugate according to statement 51, wherein the total number of carbon atoms in the R² group is no more than 4.

53. The conjugate according to statement 52, wherein the total number of carbon atoms in the R² group is no more than 3.

54. The conjugate according to any one of statements 51 to 53, wherein one of R¹¹, R¹² and R¹³ is H, with the other two groups being selected from H, C₁₋₃ saturated alkyl, C₂₋₃ alkenyl, C₂₋₃ alkynyl and cyclopropyl.

55. The conjugate according to any one of statements 51 to 53, wherein two of R¹¹, R¹² and R¹³ are H, with the other group being selected from H, C₁₋₃ saturated alkyl, C₂₋₃ alkenyl, C₂₋₃ alkynyl and cyclopropyl.

56. The conjugate according to any one of statements 12 to 41, wherein there is a double bond between C2 and C3, and R² is a group of formula:

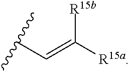

57. The conjugate according to statement 56, wherein R² is the group:

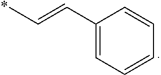

58. The conjugate according to any one of statements 12 to 41, wherein there is a double bond between C2 and C3, and R² is a group of formula:

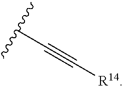

59. The conjugate according to statement 58, wherein R¹⁴ is selected from H, methyl, ethyl, ethenyl and ethynyl.

60. The conjugate according to statement 59, wherein R¹⁴ is selected from H and methyl.

61. The conjugate according to any one of statements 12 to 41, wherein there is a single bond between C2 and C3, R² is

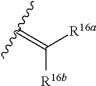

and R^{16a} and R^{16b} are both H.

62. The conjugate according to any one of statements 12 to 41, wherein there is a single bond between C2 and C3, R² is

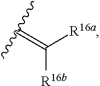

and R^{16a} and R^{16b} are both methyl.

63. The conjugate according to any one of statements 12 to 41, wherein there is a single bond between C2 and C3, R² is

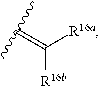

one of R^{16a} and R^{16b} is H, and the other is selected from C₁₋₄ saturated alkyl, C₂₋₃ alkenyl, which alkyl and alkenyl groups are optionally substituted.

64. The conjugate according to any one of statements 12 to 63, wherein R^{11a} is OH.

65. The conjugate according to any one of statements 12 to 64, wherein R²¹ is OH.

66. The conjugate according to any one of statements 12 to 64, wherein R²¹ is OMe.

67. The conjugate according to any one of statements 12 to 66, wherein R²⁰ is H.

68. The conjugate according to any one of statements 12 to 66, wherein R²⁰ is R^{C}.

69. The conjugate according to statement 68, wherein R^{C} is selected from the group consisting of: Alloc, Fmoc, Boc, Troc, Teoc, Psec, Cbz and PNZ.

70. The conjugate according to statement 68, wherein R^{C} is a group:

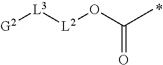

where the asterisk indicates the point of attachment to the N10 position, G² is a terminating group, L³ is a covalent bond or a cleavable linker L¹, L² is a covalent bond or together with OC(═O) forms a self-immolative linker.

71. The conjugate according to statement 70, wherein G² is Ac or Moc or is selected from the group consisting of: Alloc, Fmoc, Boc, Troc, Teoc, Psec, Cbz and PNZ.

72. The conjugate according to any one of statements 12 to 64, wherein R²⁰ and R²¹ together form a double bond between the nitrogen and carbon atoms to which they are bound.

[Formula II]

73. The conjugate according to any one of statements 12 to 41, wherein R²² is of formula IIIa, and A is phenyl.

74. The conjugate according to any one of statements 12 to 41 and statement 63, wherein R²² is of formula IIa, and Q¹ is a single bond.

75. The conjugate according to statement 73, wherein Q² is a single bond.

76. The conjugate according to statement 73, wherein Q² is —Z—(CH₂)ₙ—, Z is O or S and n is 1 or 2.

77. The conjugate according any one of statements 12 to 41 and statement 73, wherein R²² is of formula IIIa, and Q¹ is —CH═CH—.

78. The conjugate according to any one of statements 12 to 41, wherein R²² is of formula IIIb,

and R^{C1}, R^{C2} and R^{C3} are independently selected from H and methyl.

79. The conjugate according to statement 78, wherein R^{C1}, R^{C2} and R^{C3} are all H.

80. The conjugate according to statement 78, wherein R^{C1}, R^{C2} and R^{C3} are all methyl.

81. The conjugate according to any one of statements 12 to 41 and statements 73 to 80, wherein R²² is of formula IIIa or formula IIIb and X is selected from O—R^{L2′}, S—R^{L2′}, CO₂—R^{L2′}, —N—C(═O)—R^{L2′} and NH—R^{L2}′.

82. The conjugate according to statement 81, wherein X is NH—R^{L2′}.

83. The conjugate according to any one of statements 12 to 41, wherein R²² is of formula IIIc, and Q is NR^{N}—R^{L2′}.

84. The conjugate according to statement 83, wherein R^{N} is H or methyl.

85. The conjugate according to any one of statements 12 to 41, wherein R²² is of formula IIIc, and Q is O—R^{L2′} or S—R^{L2′}.

86. The conjugate according to any one of statements 12 to 41 and statements 73 to 85, wherein R¹¹ is OH.

87. The conjugate according to any one of statements 12 to 41 and statements 73 to 85, wherein R¹¹ is OMe.

88. The conjugate according to any one of statements 12 to 41 and statements 73 to 87, wherein R¹⁰ is H.

89. The conjugate according to any one of statements 12 to 41 and statements 73 to 85, wherein R¹⁰ and R¹¹ together form a double bond between the nitrogen and carbon atoms to which they are bound.

90. The conjugate according to any one of statements 12 to 41 and statements 73 to 89, wherein R³¹ is OH.

91. The conjugate according to any one of statements 12 to 41 and statements 73 to 89, wherein R³¹ is OMe.

92. The conjugate according to any one of statements 12 to 41 and statements 73 to 91, wherein R³⁰ is H.

93. The conjugate according to any one of statements 12 to 41 and statements 73 to 89, wherein R³⁰ and R³¹ together form a double bond between the nitrogen and carbon atoms to which they are bound.

94. The conjugate according to any one of statements 1 to 93, wherein R^{6′}, R^{7′}, R^{9′}, and Y′ are the same as R⁶, R⁷, R⁹, and Y.

95. The conjugate according to any one of statements 1 to 94 wherein, wherein L-R^{L1′} or L-R^{L2′} is a group:

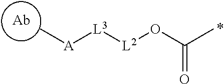

where the asterisk indicates the point of attachment to the PBD, Ab is the antibody, L¹ is a cleavable linker, A is a connecting group connecting L¹ to the antibody, L² is a covalent bond or together with —OC(═O)— forms a self-immolative linker.

96. The conjugate of statement 95, wherein L¹ is enzyme cleavable.

97. The conjugate of statement 95 or statement 96, wherein L¹ comprises a contiguous sequence of amino acids.

98. The conjugate of statement 97, wherein L¹ comprises a dipeptide and the group —X₁—X₂— in dipeptide, —NH—X₁—X₂—CO—, is selected from:
Phe-Lys-,
Val-Ala-,
Val-Lys-,
Ala-Lys-,
Val-Cit-,
Phe-Cit-,
Leu-Cit-,
Ile-Cit-,
Phe-Arg-,
Trp-Cit-.

99. The conjugate according to statement 98, wherein the group —X₁—X₂— in dipeptide, —NH—X₁—X₂—CO—, is selected from:
Phe-Lys-,
Val-Ala-,
Val-Lys-,
Ala-Lys-,
Val-Cit-.

100. The conjugate according to statement 99, wherein the group —X₁—X₂— in dipeptide, —NH—X₁—X₂—CO—, is -Phe-Lys-, -Val-Ala- or -Val-Cit-.

101. The conjugate according to any one of statements 98 to 100, wherein the group X₂—CO— is connected to L².

102. The conjugate according to any one of statements 98 to 101, wherein the group NH—X₁— is connected to A.

103. The conjugate according to any one of statements 98 to 102, wherein L² together with OC(═O) forms a self-immolative linker.

104. The conjugate according to statement 103, wherein C(═O)O and L² together form the group:

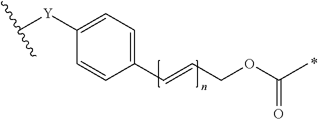

where the asterisk indicates the point of attachment to the PBD, the wavy line indicates the point of attachment to the linker L¹, Y is NH, O, C(═O)NH or C(═O)O, and n is 0 to 3.

105. The conjugate according to statement 104, wherein Y is NH.

106. The conjugate according to statement 104 or statement 105, wherein n is 0.

107. The conjugate according to statement 105, wherein L¹ and L² together with —OC(═O)— comprise a group selected from:

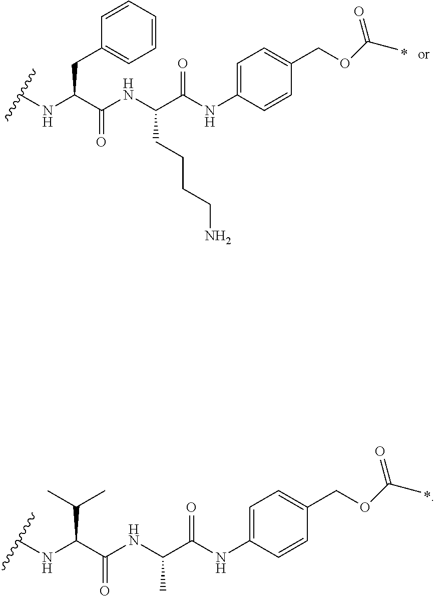

where the asterisk indicates the point of attachment to the PBD, and the wavy line indicates the point of attachment to the remaining portion of the linker L¹ or the point of attachment to A.

108. The conjugate according to statement 107, wherein the wavy line indicates the point of attachment to A.

109. The conjugate according to any one of statements 95 to 108, wherein A is:

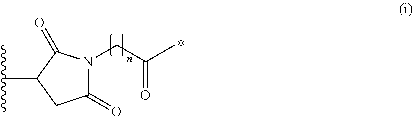

where the asterisk indicates the point of attachment to L¹, the wavy line indicates the point of attachment to the antibody, and n is 0 to 6; or

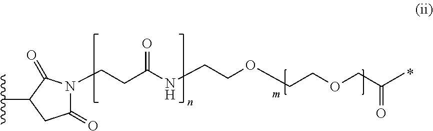

where the asterisk indicates the point of attachment to L¹, the wavy line indicates the point of attachment to the antibody, n is 0 or 1, and m is 0 to 30.

110. A conjugate according to statement 12 of formula ConjA:

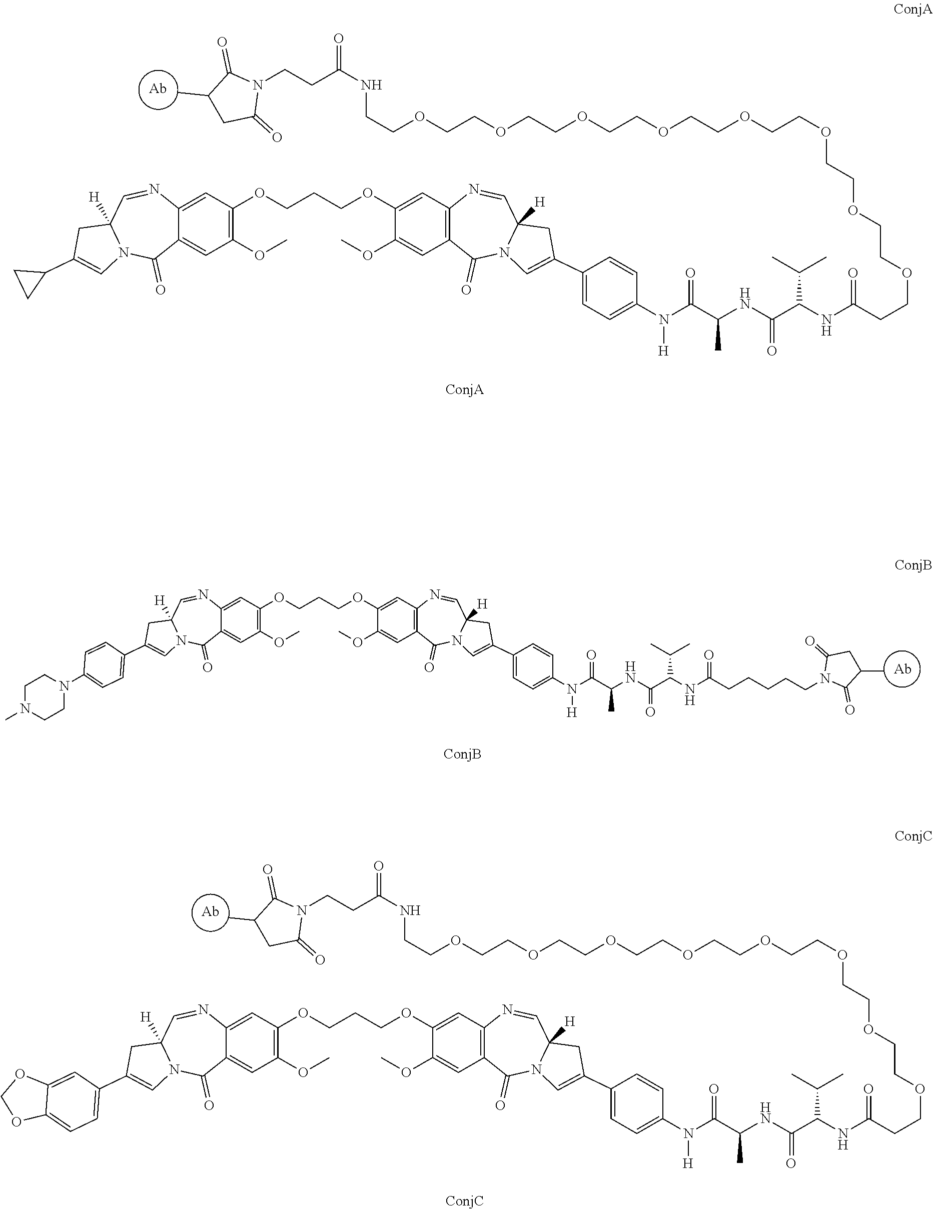

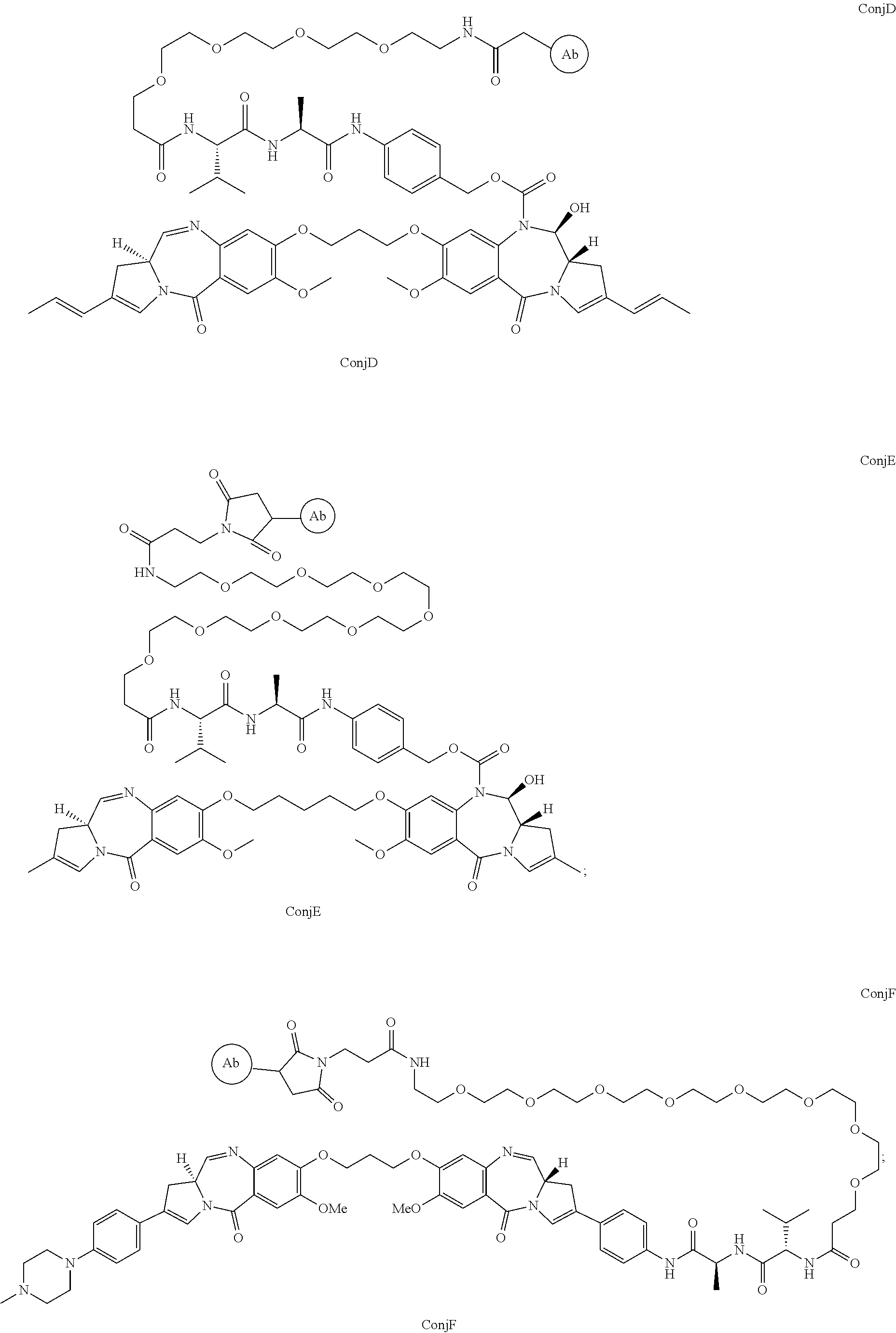

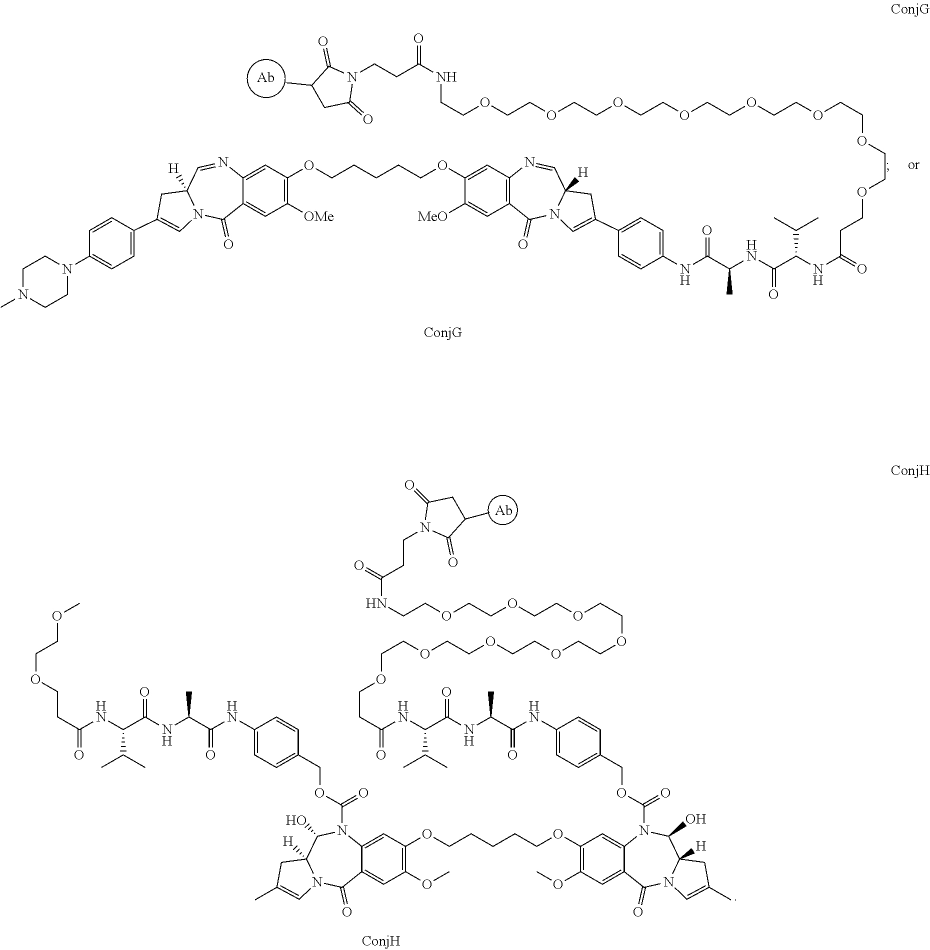

111. The drug-conjugate according to any one of statements 12 to 110 wherein the drug loading (p) of drugs (D) to antibody (Ab) is an integer from 1 to about 8.

112. The drug-conjugate according to statement 111 wherein p is 1, 2, 3, or 4.

113. The drug-conjugate according to statement 111 comprising a mixture of the antibody-drug conjugate compounds, wherein the average drug loading per antibody in the mixture of antibody-drug conjugate compounds is about 2 to about 5.

114. The antibody or drug-conjugate according to any one of statements 1 to 113, for use in therapy.

115. The antibody or drug-conjugate according to any one of statements 1 to 113, for use in the treatment of a proliferative disease in a subject.

116. The antibody or drug-conjugate according to any one of statements 1 to 113, for use in the treatment of a proliferative disease in a subject, wherein the subject has raised levels of AXL, Akt3, or GAS6 and wherein the method comprises identifying that the subject has raised levels of AXL, Akt3, or GAS6 and administering the antibody or conjugate to the patient.

117. The antibody or drug-conjugate according to any one of statements 1 to 113, for use in the treatment of a proliferative disease in a subject, wherein the proliferative disease is associated with raised levels of AXL, Akt3, or GAS6, the method comprising administering the conjugate to the patient.

118. The drug-conjugate according to any one of statements 115 to 117, wherein the disease is cancer.

119. A pharmaceutical composition comprising the antibody or drug-conjugate of any one of statements 1 to 113 and a pharmaceutically acceptable diluent, carrier or excipient.

120. The pharmaceutical composition of statement 119 further comprising a therapeutically effective amount of a chemotherapeutic agent.

121. Use of an antibody or drug-conjugate according to any one of statements 1 to 113 in the preparation of a medicament for use in the treatment of a proliferative disease in a subject.

122 A method of treating cancer comprising administering to a patient the pharmaceutical composition according to either one of statements 119 or 120.

123. The method of statement 122 wherein the patient is administered a chemotherapeutic agent, in combination with the composition.

124. A polynucleotide encoding a humanized antibody according to any one of statements 1 to 11.

125. A vector comprising the polynucleotide of statement 124.

126. The vector of statement 125 wherein the vector is an expression vector.

127. A host cell comprising a vector according to either one of statements 125 or 126.

128. The host cell according to statement 127 wherein the host cell is prokaryotic, eukaryotic, or mammalian.

129. A conjugate comprising the humanized antibody according to any one of statements 1 to 11 coupled to a functional moiety.

130. The conjugate according to statement 129, wherein the functional moiety is selected from a drug, a reporter, a toxin, an organic moiety, and a binding member.

131. The conjugate according to statement 130 wherein the reporter is a fluorescent compound, a radionuclide, or an enzyme.

132. The conjugate according to statement 130 wherein the binding member is an antibody or antibody fragment.

133. The conjugate according to any one of statements 129 to 130, wherein the humanized antibody is covalently bonded to the functional moiety.

134. A method of selecting an individual for treatment with the antibody or drug-conjugate according to any one of statements 1 to 113, or with the pharmaceutical composition of either one of statements 119 or 120, which method comprises assessing the level of AXL;
wherein individuals having raised levels of AXL are selected for treatment.

135. A method of timing the application of treatment of an individual with the abntibody or drug-conjugate according to any one of statements 1 to 113, or with the pharmaceutical composition of either one of statements 119 or 120, which method comprises assessing the level of AXL;
wherein the treatment is applied if the individual has raised levels of AXL.

136. The method according to either one of statements 134 or 135, wherein the individual has cancer and treatment reduces tumour volume.

SEQUENCES
[1H12VH]
SEQ ID NO: 1
MGFKMESQFQVFVFVFLWLSGVDGEVQLVESGGDLVKPGGSLKLSCAAS
GFTFSSYGMSWVRQTPDKRLEWVATISSGGSYTYYPDSVKGRFTISRDN
AKNTLYLQMSSLKSEDTAMYYCARHPIYYTYDDTMDYWGQGTSVTVSS
[1H12RHA]
SEQ ID NO: 2
MGFKMESQFQVFVFVFLWLSGVDGQVQLVESGGGVVQPGRSLRLSCAAS
GFTFSSYGMSVRQAPGKGLEWVATISSGGSYTYYPDSVKGRFTISRDNS
KNTLYLQMNSLRAEDTAVYYCARHPIYYTYDDTMDYWGQGTTVTVSS
[1H12RHB]
SEQ ID NO: 3
MGFKMESQFQVFVFVFLWLSGVDGEVQLVESGGGLVQPGGSLRLSCAAS
GFTFSSYGMSWVRQAPGKGLEWVATISSGGSYTYYPDSVKGRFTISRDN
AKNSLYLQMNSLRAEDTAVYYCARHPIYYTYDDTMDYWGQGTLVTVSS
[1H12VK]
SEQ ID NO: 4
MGFKMESQFQVFVFVFLWLSGVDGENVLTQSPAIMAASPGEKVTMTCSA
SSSVSSGNFHWYQQKPGTSPKLWIYRTSNLASGVPARFSGSGSGTSYSL
TISSMEAEDAATYYCQQWSGYPWTFGGGTKLEIK
[1H12RKA]
SEQ ID NO: 5
MGFKMESQFQVFVFVFLWLSGVDGEIVLTQSPATLSLSPGERATLSCSA
SSSVSSGNFHWYQQKPGLAPRLLIYRTSNLASGIPDRFSGSGSGTDFTL
TISRLEPEDFAVYYCQQWSGYPWTFGPGTKVDIK
[1H12RKA1]
SEQ ID NO: 6
MGFKMESQFQVFVFVFLWLSGVDGENVLTQSPATLSLSPGERATLSCSA
SSSVSSGNFHWYQQKPGLAPRLWIYRTSNLASGIPDRFSGSGSGTDYTL
TISRLEPEDFAVYYCQQWSGYPWTFGPGTKVDIK
[1H12RKB]
SEQ ID NO: 7
MGFKMESQFQVFVFVFLWLSGVDGEIVLTQSPGTLSLSPGERATLSCSA
SSSVSSGNFHWYQQKPGLAPRLLIYRTSNLASGIPARFSGSGSGTDFTL
TISSLEPEDFAVYYCQQWSGYPWTFGGGTKLEIK
[1H12RKB1]
SEQ ID NO: 8
MGFKMESQFQVFVFVFLWLSGVDGENVLTQSPGTLSLSPGERATLSCSA
SSSVSSGNFHWYQQKPGLAPRLWIYRTSNLASGIPARFSGSGSGTDYTL
TISSLEPEDFAVYYCQQWSGYPWTFGGGTKLEIK
[Human Axl]
SEQ ID NO: 9
MAWRCPRMGRVPLAWCLALCGWACMAPRGTQAEESPFVGNPGNITGARG
LTGTLRCQLQVQGEPPEVHWLRDGQILELADSTQTQVPLGEDEQDDWIV
VSQLRITSLQLSDTGQYQCLVFLGHQTFVSQPGYVGLEGLPYFLEEPED
RTVAANTPFNLSCQAQGPPEPVDLLWLQDAVPLATAPGHGPQRSLHVPG
LNKTSSFSCEAHNAKGVTTSRTATITVLPQQPRNLHLVSRQPTELEVAW
TPGLSGIYPLTHCTLQAVLSDDGMGIQAGEPDPPEEPLTSQASVPPHQL
RLGSLHPHTPYHIRVACTSSQGPSSWTHWLPVETPEGVPLGPPENISAT
RNGSQAFVHWQEPRAPLQGTLLGYRLAYQGQDTPEVLMDIGLRQEVTLE
LQGDGSVSNLTVCVAAYTAAGDGPWSLPVPLEAWRPGQAQPVHQLVKEP
STPAFSWPWWYVLLGAVVAAACVLILALFLVHRRKKETRYGEVFEPTVE
RGELVVRYRVRKSYSRRTTEATLNSLGISEELKEKLRDVMVDRHKVALG
KTLGEGEFGAVMEGQLNQDDSILKVAVKTMKIAICTRSELEDFLSEAVC
MKEFDHPNVMRLIGVCFQGSERESFPAPVVILPFMKHGDLHSFLLYSRL
GDQPVYLPTQMLVKFMADIASGMEYLSTKRFIHRDLAARNCMLNENMSV
CVADFGLSKKIYNGDYYRQGRIAKMPVKWIAIESLADRVYTSKSDVWSF
GVTMWEIATRGQTPYPGVENSEIYDYLRRGNRLKQPADCLDGLYALMSR
CWELNPQDRPSFTELREDLENTLKALPPAQEPDEILYVNMDEGGGYPEP
PGAAGGADPPTQPDPKDSCSCLTAAEVHPAGRYVLCPSTTPSPAQPADR
GSPAAPGQEDGA
[Murine Axl]
SEQ ID NO: 10
MGRVPLAWWLALCCWGCAAHKDTQTEAGSPFVGNPGNITGARGLTGTLR
CELQVQGEPPEVVWLRDGQILELADNTQTQVPLGEDWQDEWKVVSQLRI
SALQLSDAGEYQCMVHLEGRTFVSQPGFVGLEGLPYFLEEPEDKAVPAN
TPFNLSCQAQGPPEPVTLLWLQDAVPLAPVTGHSSQHSLQTPGLNKTSS
FSCEAHNAKGVTTSRTATITVLPQRPHHLHVVSRQPTELEVAWTPGLSG
IYPLTHCNLQAVLSDDGVGIWLGKSDPPEDPLTLQVSVPPHQLRLEKLL
PHTPYHIRISCSSSQGPSPWTHWLPVETTEGVPLGPPENVSAMRNGSQV
LVRWQEPRVPLQGTLLGYRLAYRGQDTPEVLMDIGLTREVTLELRGDRP
VANLTVSVTAYTSAGDGPWSLPVPLEPWRPGQGQPLHHLVSEPPPRAFS
WPWWYVLLGALVAAACVLILALFLVHRRKKETRYGEVFEPTVERGELVV
RYRVRKSYSRRTTEATLNSLGISEELKEKLRDVMVDRHKVALGKTLGEG
EFGAVMEGQLNQDDSILKVAVKTMKIAICTRSELEDFLSEAVCMKEFDH
PNVMRLIGVCFQGSDREGFPEPVVILPFMKHGDLHSFLLYSRLGDQPVF
LPTQMLVKFMADIASGMEYLSTKRFIHRDLAARNCMLNENMSVCVADFG
LSKKIYNGDYYRQGRIAKMPVKWIAIESLADRVYTSKSDVWSFGVTMWE
IATRGQTPYPGVENSEIYDYLRQGNRLKQPVDCLDGLYALMSRCWELNP
RDRPSFAELREDLENTLKALPPAQEPDEILYVNMDEGGSHLEPRGAAGG
ADPPTQPDPKDSCSCLTAADVHSAGRYVLCPSTAPGPTLSADRGCPAPP
GQEDGA

1.-126. (canceled)

127. A conjugate of formula L-(DL)p, where DL is of formula I or II: 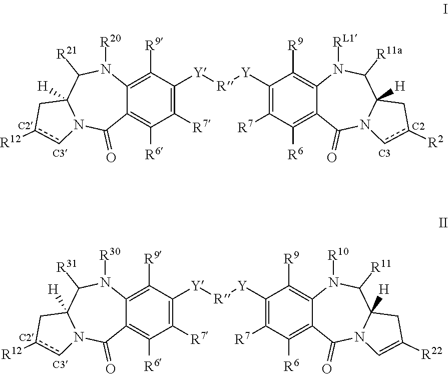
wherein:
L is an isolated humanized antibody (Ab) that binds to AXL, wherein the isolated humanized antibody comprises a heavy chain variable region having the amino acid sequence of SEQ ID NO: 2 or SEQ ID NO: 3;
when there is a double bond present between C2′ and C3′, R¹² is selected from the group consisting of:
(ia) C₅₋₁₀ aryl group, optionally substituted by one or more substituents selected from the group comprising: halo, nitro, cyano, ether, carboxy, ester, C₁₋₇ alkyl, CO₃₋₇ heterocyclyl and bis-oxy-C₁₋₃ alkylene;
(ib) C₁₋₅ saturated aliphatic alkyl;
(ic) C₃₋₆ saturated cycloalkyl; 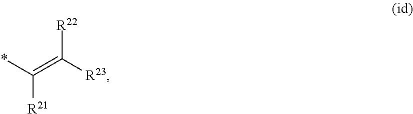
wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5; 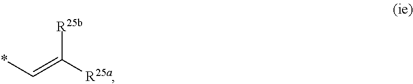
wherein one of R^{25a} and R^{25b} is H and the other is selected from: phenyl, which phenyl is optionally substituted by a group selected from halo, methyl, methoxy; pyridyl; and thiophenyl; and 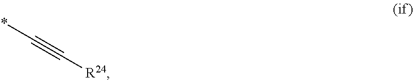
where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2′ and C3′,
R¹² is 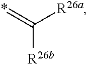
where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
R⁶ and R⁹ are independently selected from H, R, OH, OR, SH, SR, NH₂, NHR, NRR′, nitro, Me₃Sn and halo;
where R and R′ are independently selected from optionally substituted C₁₋₁₂ alkyl, C₃₋₂₀ heterocyclyl and C₅₋₂₀ aryl groups;
R⁷ is selected from H, R, OH, OR, SH, SR, NH₂, NHR, NHRR′, nitro, Me₃Sn and halo;
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine; Y and Y′ are selected from O, S, or NH;
R^{6′}, R^{7′}, R^{9′} are selected from the same groups as R⁶, R⁷ and R⁹ respectively;
[Formula I]
R^{L1′} is a linker for connection to the antibody (Ab);
R^{11a} is selected from OH, OR^{A}, where R^{A} is C₁₋₄ alkyl, and SO_{z}M, where z is 2 or 3 and M is a monovalent pharmaceutically acceptable cation;
R²⁰ and R²¹ either together form a double bond between the nitrogen and carbon atoms to which they are bound or;
R²⁰ is selected from H and R^{C}, where R^{C} is a capping group;
R²¹ is selected from OH, OR^{A} and SO_{z}M;
when there is a double bond present between C2 and C3, R² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl; 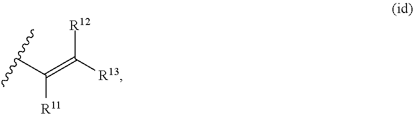
wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5; 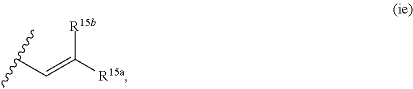
wherein one of R^{15a} and R^{15b} is H and the other is selected from: phenyl, which phenyl is optionally substituted by a group selected from halo, methyl, methoxy; pyridyl; and thiophenyl; and 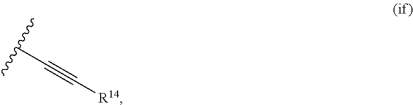
where R¹⁴ is selected from: H; C1-3 saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2 and C3,
R² is 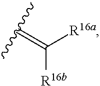
where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;
[Formula II]
R²² is of formula IIIa, formula IIIb or formula IIIc: 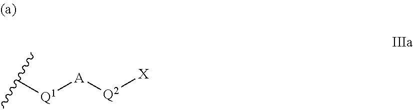
where A is a C₅₋₇ aryl group, and either
(i) Q¹ is a single bond, and Q² is selected from a single bond and —Z—(CH₂)ₙ—, where Z is selected from a single bond, O, S and NH and n is from 1 to 3; or
(ii) Q¹ is —CH═CH—, and Q² is a single bond; 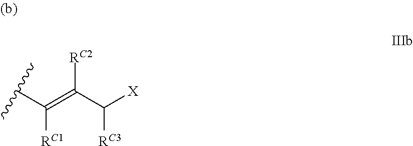
where;
R^{C1}, R^{C2} and R^{C3} are independently selected from H and unsubstituted C₁₋₂ alkyl; 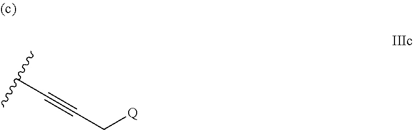
where Q is selected from O—R^{L2′}, S—R^{L2′} and NR^{N}—R^{L2′},and R^{N} is selected from H, methyl and ethyl
X is selected from the group comprising: O—R^{L2′}, S—R^{L2′}, CO₂—R^{L2′}, CO—R^{L2′}, NH—C(═O)—R^{L2′}, NHNH—R^{L2′}, CONHNH—R^{L2′}, 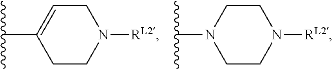
NR^{N}R^{L2′}, wherein R^{N} is selected from the group comprising H and C₁₋₄ alkyl;
R^{L2′} is a linker for connection to the antibody (Ab);
R¹⁰ and R¹¹ either together form a double bond between the nitrogen and carbon atoms to which they are bound or;
R¹⁰ is H and R¹¹ is selected from OH, OR^{A} and SO_{z}M;
R³⁰ and R³¹ either together form a double bond between the nitrogen and carbon atoms to which they are bound or;
R³⁰ is H and R³¹ is selected from OH, OR^{A} and SO_{z}M.

128. The conjugate of claim 127, wherein R⁷ is a C₁₋₄ alkyloxy group.

129. The conjugate of claim 127, wherein Y is O and R″ is C₃₋₇ alkylene.

130. The conjugate of claim 127, wherein R⁶ and R⁹ are H.

131. The conjugate of claim 127, wherein there is a double bond between C2′ and C3′, and R¹² is:
(a) a C₅₋₇ aryl group, which may bear one to three substituent groups selected from methoxy, ethoxy, fluoro, chloro, cyano, bis-oxy-methylene, methyl-piperazinyl, morpholino and methyl-thiophenyl; or
(b) methyl, ethyl or propyl; or
(c) cyclopropyl; or
(d) a group of formula: 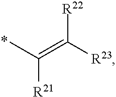
wherein the total number of carbon atoms in the R¹² group is no more than 4; or
(e) the group: 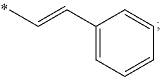
or
(f) a group of formula: 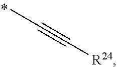
wherein R²⁴ is selected from H and methyl.

132. The conjugate of claim 127, wherein there is a single bond between C2′ and C3′,
R¹² is 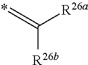
and:
(a) R^{26a} and R^{26b} are both H; or
(b) R^{26a} and R^{26b} are both methyl; or
(c) one of R^{26a} and R^{26b} is H, and the other is selected from C₁₋₄ saturated alkyl, C₂₋₃ alkenyl, which alkyl and alkenyl groups are optionally substituted.
[Formula I]

133. The conjugate of claim 127, wherein there is a double bond between C2 and C3, and R² is:
(a) a C₅₋₇ aryl group, which may bear one to three substituent groups selected from methoxy, ethoxy, fluoro, chloro, cyano, bis-oxy-methylene, methyl-piperazinyl, morpholino and methyl-thiophenyl; or
(b) methyl, ethyl or propyl; or
(c) cyclopropyl; or
(d) a group of formula: 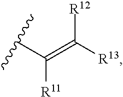
wherein the total number of carbon atoms in the R² group is no more than 4; or
(e) the group: 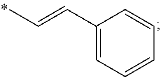
or
(f) a group of formula: 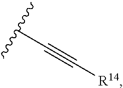
wherein R¹⁴ is selected from H and methyl.

134. The conjugate of claim 127, wherein there is a single bond between C2 and C3,
R² is 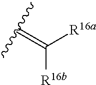
and:
(a) R^{16a} and R^{16b} are both H; or
(b) R^{16a} and R^{16b} are both methyl; or
(c) one of R^{16a} and R^{16b} is H, and the other is selected from C₁₋₄ saturated alkyl, C₂₋₃ alkenyl, which alkyl and alkenyl groups are optionally substituted.

135. The conjugate of claim 127, wherein R²⁰ is R^{C}, wherein R^{C} is a group: 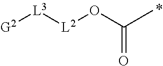
where the asterisk indicates the point of attachment to the N10 position, G² is a terminating group, L³ is a covalent bond or a cleavable linker L¹, L² is a covalent bond or together with OC(═O) forms a self-immolative linker.
[Formula II]

136. The conjugate of claim 127, wherein:
(a) R²² is of formula IIIa, A is phenyl, Q¹ is a single bond, Q² is a single bond; or
(b) R²² is of formula IIIb, and R^{C1}, R^{C2} and R^{C3} are all H;
and X is NH—R^{L2′}.

137. The conjugate of claim 127, wherein R^{6′}, R^{7′}, R^{9′}, and Y′ are the same as R⁶, R⁷, R⁹, and Y.

138. The conjugate of claim 127, wherein L-R^{L1′} Or L-R^{L2′} is a group: 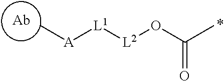
where the asterisk indicates the point of attachment to the PBD, Ab is the antibody, L¹ is a cleavable linker, A is a connecting group connecting L¹ to the antibody, L² is a covalent bond or together with —OC(═O)— forms a self-immolative linker.

139. The conjugate of claim 138, wherein L¹ comprises a dipeptide and the group —X₁—X₂— in dipeptide, —NH—X₁—X₂—CO—, is selected from:
Phe-Lys-,
Val-Ala-,
Val-Lys-,
Ala-Lys-,
Val-Cit-.

140. The conjugate of claim 139, wherein C(═O)O and L² together form the group: 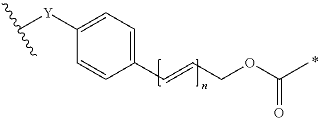
where the asterisk indicates the point of attachment to the PBD, the wavy line indicates the point of attachment to the linker L¹, Y is NH, O, C(═O)NH or C(═O)O, and n is 0 to 3.

141. A conjugate of claim 127 of formula

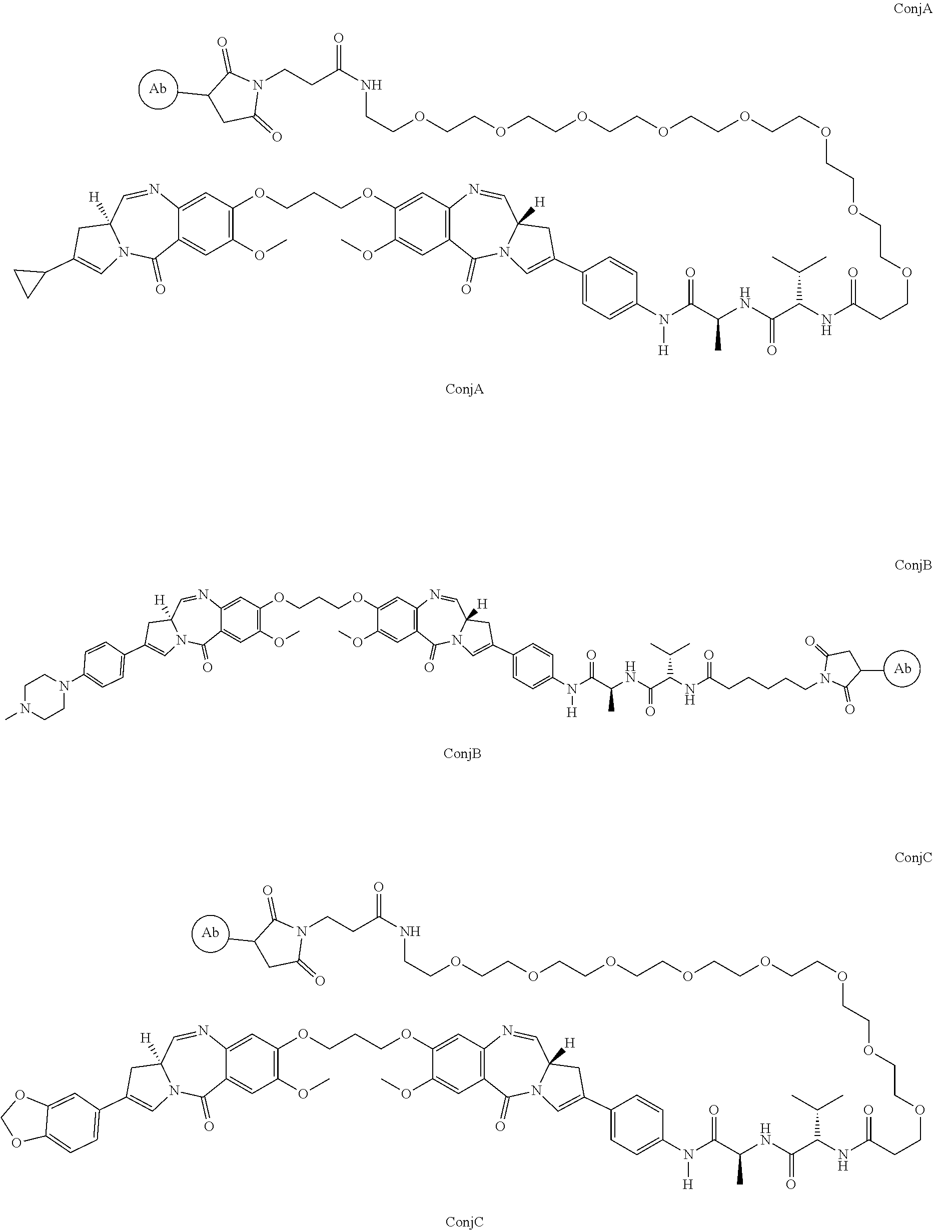

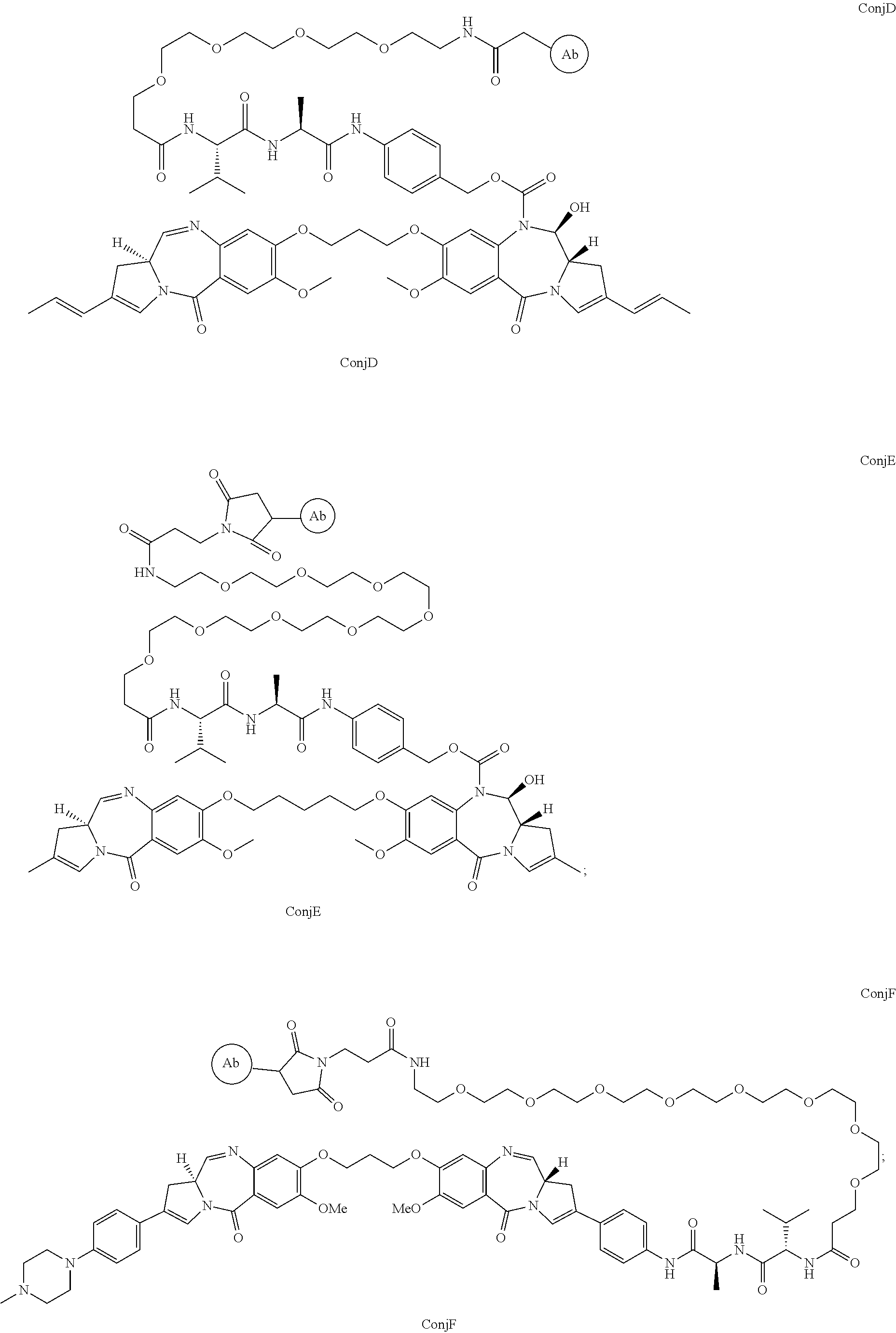

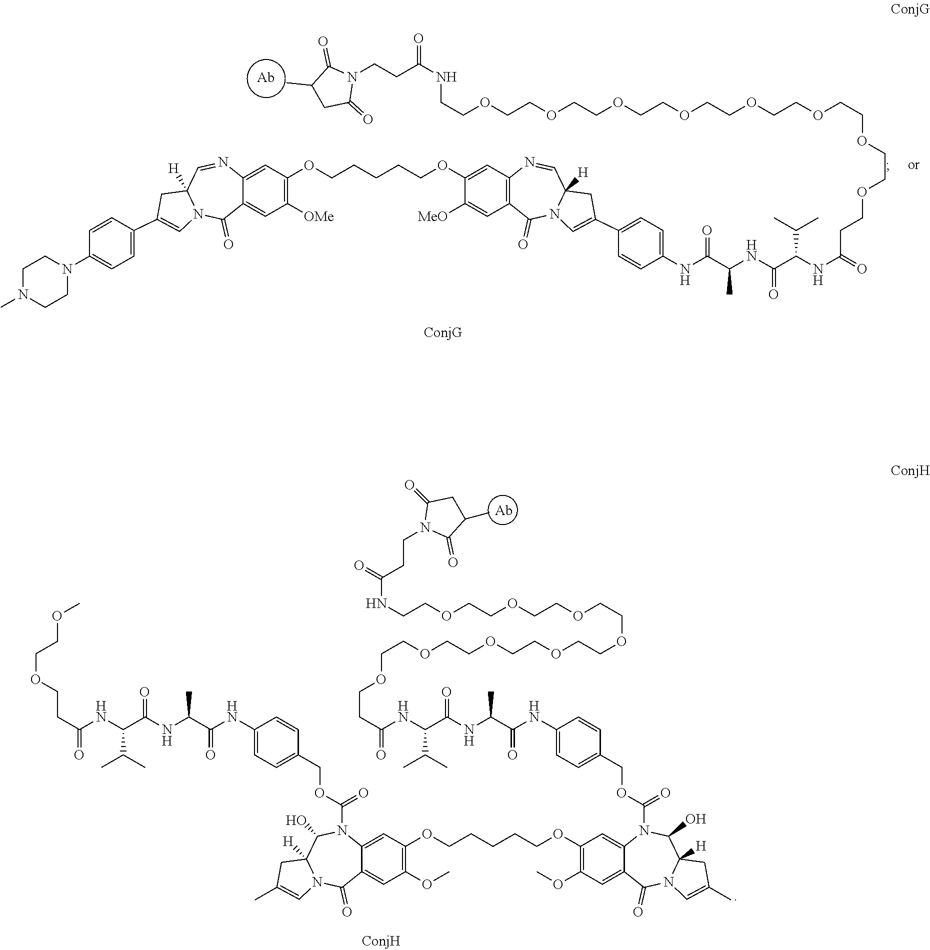

142. The conjugate of claim 127, wherein the isolated humanized antibody further comprises a light chain variable region having the amino acid sequence of SEQ ID NO: 4, 5, 6, 7, or 8; and, optionally,
comprises a constant region derived from one or more human antibodies.

143. The conjugate of claim 127, wherein the isolated humanized antibody comprises:
(i) a heavy chain variable region having the amino acid sequence of SEQ ID NO: 2 and a light chain variable region having the amino acid sequence of SEQ ID NO: 4;
(ii) a heavy chain variable region having the amino acid sequence of SEQ ID NO: 2 and a light chain variable region having the amino acid sequence of SEQ ID NO: 5;
(iii) a heavy chain variable region having the amino acid sequence of SEQ ID NO: 2 and a light chain variable region having the amino acid sequence of SEQ ID NO: 6;
(iv) a heavy chain variable region having the amino acid sequence of SEQ ID NO: 2 and a light chain variable region having the amino acid sequence of SEQ ID NO: 7;
(v) a heavy chain variable region having the amino acid sequence of SEQ ID NO: 2 and a light chain variable region having the amino acid sequence of SEQ ID NO: 8;
(vi) a heavy chain variable region having the amino acid sequence of SEQ ID NO: 3 and a light chain variable region having the amino acid sequence of SEQ ID NO: 4;
(vii) a heavy chain variable region having the amino acid sequence of SEQ ID NO: 3 and a light chain variable region having the amino acid sequence of SEQ ID NO: 5;
(viii) a heavy chain variable region having the amino acid sequence of SEQ ID NO: 3 and a light chain variable region having the amino acid sequence of SEQ ID NO: 6;
(viv) a heavy chain variable region having the amino acid sequence of SEQ ID NO: 3 and a light chain variable region having the amino acid sequence of SEQ ID NO: 7; or
(x) a heavy chain variable region having the amino acid sequence of SEQ ID NO: 3 and a light chain variable region having the amino acid sequence of SEQ ID NO: 8.

144. The conjugate of claim 127, wherein said antibody competitively inhibits the binding to human AXL of an antibody comprising a heavy chain variable region having the amino acid sequence of SEQ ID NO: 1 and a light chain variable region having the amino acid sequence of SEQ ID NO: 4.

145. The conjugate of claim 127, wherein said antibody or antibody fragment has a constant region of either isotype IgG1, IgG2, IgG3 or IgG4, or a mutated IgG constant region, and optionally a light chain constant region of isotype kappa or lambda.

146. The conjugate of claim 145, wherein p is 1, 2, 3, or 4.

147. The conjugate of claim 145, comprising a mixture of the antibody-drug conjugate compounds, wherein the average drug loading per antibody in the mixture of antibody-drug conjugate compounds is about 2 to about 5.

148. The conjugate of claim 127, for use in therapy.

149. The conjugate of claim 127, for use in the treatment of a proliferative disease in a subject.

150. The conjugate of claim 149, wherein the disease is cancer.

151. A pharmaceutical composition comprising the conjugate of claim 127 and a pharmaceutically acceptable diluent, carrier or excipient.

152. The pharmaceutical composition of claim 151, further comprising a therapeutically effective amount of a chemotherapeutic agent.

153. A method of selecting an individual for treatment with the conjugate of claim 127, which method comprises assessing the level of AXL; wherein individuals having raised levels of AXL are selected for treatment.

154. A method of timing the application of treatment of an individual with the conjugate of claim 127, which method comprises assessing the level of AXL; wherein the treatment is applied if the individual has raised levels of AXL.

155. The method according to claim 153, wherein the individual has cancer and treatment reduces tumour volume.
